(19) United States
(12) Patent Application Publication
(10) Pub. No.: US 2025/0041125 A1
(43) Pub. Date: Feb. 6, 2025

(54) WEDGE CUSHIONING SYSTEMS

(71) Applicant: Sage Products, LLC, Cary, IL (US)

(72) Inventors: Catherine S. Boulos, Cary, IL (US); Ronald C. Cagle, Crystal Lake, IL (US); David P. Beck, Crystal Lake, IL (US)

(21) Appl. No.: 18/782,307

(22) Filed: Jul. 24, 2024

Related U.S. Application Data
(60) Provisional application No. 63/529,874, filed on Jul. 31, 2023.

(51) Int. Cl. A61F 13/06; A61B 5/00

(52) U.S. Cl. CPC A61F 13/069; A61B 5/447; A61B 5/7275; A61B 5/746

(57) ABSTRACT

A wedge cushioning system for supporting a lower limb of a patient includes a wedge assembly. The wedge assembly includes a wedge pad configured to cushion the lower limb of the patient, a first wedge wing pivotably coupled to a first side of the wedge pad and selectively pivotable between a first lowered position and a first raised position, a second wedge wing pivotably coupled to a second side of the wedge pad and selectively pivotably between a second lowered position and a second raised position independent of the first wedge wing, and a strap configured to be releasably coupled between the first wedge wing and the second wedge wing. The second side of the wedge pad is opposite the first side of the wedge pad.

### CROSS-REFERENCE TO RELATED APPLICATION

This patent application claims the benefit of and priority to U.S. Provisional Patent Application No. 63/529,874, filed Jul. 31, 2023, the entire disclosure of which is hereby incorporated by reference herein.

### BACKGROUND

Caregivers may outfit or provide patients positioned on a bed with structures to provide comfort to the patients and to protect the patients from effects of the patient being positioned on the bed. For example, caregivers may outfit or provide the patients with structures configured to increase the comfort of the patients and protect the patients from pressure injuries. Some pressure injuries may, for example, occur due to prolonged pressure on a patient's skin from a bed. Further, the caregivers may outfit or provide the patients with boots and/or foot wraps to protect the heels of the feet of the patients from developing pressure injuries on the heels. However, the boots and/or foot wraps may be difficult for the caregivers to outfit on the patients. For example, when a caregiver positions the heel of a patient on a wedge cushion with a slippery surface, the wedge cushion may slip out from under the foot of the patient.

### SUMMARY

In one embodiment, a wedge cushioning system for supporting a lower limb of a patient includes a wedge assembly. The wedge assembly includes a wedge pad configured to cushion the lower limb of the patient, a first wedge wing pivotably coupled to a first side of the wedge pad and selectively pivotable between a first lowered position and a first raised position, a second wedge wing pivotably coupled to a second side of the wedge pad and selectively pivotably between a second lowered position and a second raised position independent of the first wedge wing, and a strap configured to be releasably coupled between the first wedge wing and the second wedge wing. The second side of the wedge pad is opposite the first side of the wedge pad.

In another embodiment, a wedge cushioning system for supporting a lower limb of a patient includes a wedge assembly configured to cushion the lower limb of the patient. The wedge assembly includes a wedge top surface. The wedge cushioning system also includes a sensor positioned on the wedge assembly configured to generate sensor data associated with the wedge assembly and a processing circuit. The processing circuit includes memory and one or more processors. The processing circuit is configured to receive, from the sensor, the sensor data associated with the wedge assembly, determine, based on the sensor data, a pressure applied on the wedge top surface of the wedge assembly, and provide the pressure applied on the wedge top surface to a user interface associated with a caregiver.

### BRIEF DESCRIPTION OF THE DRAWINGS

The details of one or more implementations are set forth in the accompanying drawings and the description below. Other features, aspects, and advantages of the disclosure will become apparent from the description, the drawings, and the claims, in which:

FIG. 1 is a side perspective view of an example cushioning boot;

FIG. 2 is a top view of the cushioning boot of FIG. 1;

FIG. 3 is a side view of the cushioning boot of FIG. 1;

FIGS. 4A-4B depict locations of mechanical stimulation provided to a patient by an example mechanical stimulation system;

FIG. 5 is a perspective view of an example compression system;

FIG. 6 is another perspective view of another example compression device;

FIG. 7 is another perspective view of another example compression device;

FIG. 8 is a schematic representation of one of the compression devices of FIGS. 5-7;

FIG. 9 is a back view of a portion one of the compression devices of FIGS. 5-7;

FIG. 10 is a side view of an example boot cover;

FIG. 11 is a side view of an example universal fit positioning system;

FIG. 12 is a perspective view of the universal fit positioning system of FIG. 11;

FIG. 13 is an exploded view of the universal fit positioning system of FIG. 11;

FIG. 14 is perspective view of a portion of the universal fit positioning system of FIG. 11 supporting a limb of a patient;

FIG. 15 is side perspective view of the universal fit positioning system of FIG. 11 supporting another limb of a patient;

FIG. 16 is top perspective view of the universal fit positioning system of FIG. 11 supporting another limb of a patient;

FIG. 17 is a perspective view of an example modular positioning system supporting a limb of a patient;

FIG. 18 is side perspective view of the modular positioning system of FIG. 17 supporting the limb of the patient;

FIG. 19 is a top view of an example wedge cushioning system;

FIG. 20 is a perspective view of an example wedge assembly of the wedge cushioning system of FIG. 19;

FIG. 21 is a top view of the wedge assembly of FIG. 20;

FIG. 22 is a bottom perspective view of another example wedge assembly of the wedge cushioning system of FIG. 19;

FIG. 23 is a top view of the wedge assembly of FIG. 22;

FIG. 24 is a top view of an example strap holding together wings of the wedge assembly of FIG. 22;

FIG. 25 is a top view of another example strap holding together the wings of the wedge assembly of FIG. 22;

FIG. 26 is a perspective view of an example support matrix of the wedge assembly of FIG. 20 or the wedge assembly of FIG. 22;

FIG. 27 is a top view of the support matrix of FIG. 26;

FIG. 28 is a top view of an example heel wrap assembly of the wedge cushioning system of FIG. 19;

FIG. 29 is a top view of an example grip element of the wedge cushioning system of FIG. 19;

FIG. 30A is a bottom view of an example dressing boot assembly;

FIG. 30B is a top view of the dressing boot assembly of FIG. 30A;

FIGS. 31A-31C are perspective views of the dressing boot assembly of FIG. 30A positioned on a limb of a patient;

FIG. 32 is a perspective view of another example dressing boot assembly positioned on a limb of a patient;

FIG. 33A is a top view of another example dressing boot assembly;

FIGS. 33B-33D are perspective views of the dressing boot assembly of FIG. 33A positioned on a limb of a patient;

FIG. 34 is a side view of an example foot drop assembly;

FIG. 35 is a top view of the foot drop assembly of FIG. 34;

FIG. 36 is a bottom view of the foot drop assembly of FIG. 34;

FIG. 37 is a side view of the foot drop assembly of FIG. 34 positioned on a limb of a patient;

FIG. 38 is a top view of the foot drop assembly of FIG. 34 positioned on the limb of the patient;

FIG. 39 is another side view of the foot drop assembly of FIG. 34 positioned on the limb of the patient;

FIG. 40 is another side view of the foot drop assembly of FIG. 34 positioned on the limb of the patient;

FIG. 41 is another top view of the foot drop assembly of FIG. 34 positioned on the limb of the patient;

FIG. 42 is a side perspective view of the foot drop assembly of FIG. 34 positioned on the limb of the patient;

FIG. 43A is a top view of an example temperature regulation system;

FIG. 43B is a bottom perspective view of the temperature regulation system of FIG. 43A formed in a boot shape;

FIG. 43C is a top perspective view of the temperature regulation system of FIG. 43A formed in a boot shape;

FIG. 44 is a bottom view of an example sensor of the temperature regulation system of FIG. 43A;

FIG. 45 is a side view of an example prone cushioning assembly used with the cushioning boot of FIG. 1;

FIG. 46 is a side view of the prone cushioning assembly of FIG. 45; and

FIG. 47 is a block diagram of an example control system.

### DETAILED DESCRIPTION

Before turning to the figures, which illustrate certain example embodiments in detail, it should be understood that the present disclosure is not limited to the details or methodology set forth in the description or illustrated in the figures. It should also be understood that the terminology used herein is for the purpose of description only and should not be regarded as limiting.

### I. Overview

Some lower limb positioning systems (e.g., limb positioning systems, limb positioning devices, limb positioning mechanisms, limb protection systems) include a boot (e.g., shoe, footwear, a foot protecting boot, a pressure relief boot, a pressure offloading boot, a pressure ulcer boot, a dressing boot) configured to be placed (e.g., positioned, dressed) by a caregiver (e.g., a doctor, a nurse, a health care worker, a clinician) on a lower limb (e.g., a leg) or a portion of the lower limb (e.g., a foot, a heel, a shin, a calf) of a patient (e.g., client, person, individual) positioned on a surface (e.g., a bed, an exam table, an operating table) between the lower limb or the portion of the lower limb and the surface. The lower limb positioning system may increase a comfort (e.g., a relaxation, reduce pain of) the patient and/or protect the lower limb of the patient while the patient is positioned on the surface. The lower limb positioning system may protect the lower limb of the patient from pressure injuries (e.g., bed sores, pressure ulcers) that occur when there is prolonged pressure applied on the lower limb of the patient from the surface that the patient is positioned on. A caregiver may place the lower limb position system on the lower limb or the portion of the lower limb of the patient when the patient is bedridden (e.g., on bedrest, has difficultly moving on the surface) to increase the comfort of the patient and/or protect the lower limb of the patient.

According to some estimates, about five percent of hospitalized patients in an intensive care unit develop a pressure injury during their stay in the hospital, and of these, about 35.5% of stage I pressure injuries advance to stage II or greater. The higher the severity (e.g., stage) of pressure injury, the longer the length of stay in the hospital. According to some estimates, the average length of stay in the hospital for pressure injuries is about 10 days for stage I, about 24 days for stage II, about 136 days for stage III, and about 223 days for stage IV with significant costs associated with each day of stay. Pressure injuries are considered “never events” in some countries such as the United States, and hospitals in these countries are not typically reimbursed for treatment related to these injuries incurred during care of a patient.

When the lower limb positioning systems are placed on the lower limb of the patient, a caregiver may secure the lower limb positioning system to the lower limb of the patient. For example, the caregiver may engage (e.g., wrap, tie) a strap (e.g., a band, a rope) around the lower limb position system to secure the lower limb position system in an optimal configuration. However, because the lower limb positioning systems typically can move (e.g., slip, slide) relative to the surface and/or the lower limb of the patient, the lower limb positioning systems may move after installation into a non-optimal configuration (e.g., an uninstalled configuration). As a result of the lower limb positioning system being in the non-optimal configuration, the patient may be uncomfortable (e.g., in discomfort) and/or may be at risk of being injured (e.g., developing pressure injuries).

Additionally, when the lower limb positioning systems are placed on the lower limb of the patient, the lower limb positioning systems may reduce a probability of pressure injuries occurring to the lower limb of the patient. For example, the lower limb positioning systems may distribute the force applied by the surface where the patient is positioned such that the pressure applied on the patient by the surface is reduced. However, if the lower limb position systems are left on the lower limb of the patient for a significant amount of time (e.g., days, weeks) without the lower limb of the patient being stimulated (e.g., massaged, compressed, moved), pressure injuries may still develop on the lower limb of the patient.

Additionally, when the lower limb positioning systems are placed on the lower limb of the patient, it may be difficult for the patient to ambulate (e.g., walk, utilize the lower limb). During ambulation, the lower limb position systems may, for example, be positioned between a foot of the lower limb of the patient and the ground. This positioning could disrupt the ambulation of the patient by introducing imbalance to the patient or a lack of traction between the lower limb position system and the ground. As a result of the disruption to the ambulation of the patient, the lower limb positioning systems may need to be removed prior to ambulation of the patient, the patient choose not to ambulate with the lower limb position system installed, and/or the patient may become a fall-risk while ambulating with the lower limb positioning system installed.

Additionally, the lower limb positioning systems may be disposable systems (e.g., single use systems) configured to be disposed (e.g., thrown away, trashed) after every use. For example, the lower limb positioning systems may be disposable foot wraps that are designed to be thrown away (e.g., disposed of) after each use. As a result of the lower limb positioning systems being disposable systems, significant waste (e.g., trash, landfill material) may be generated by the process of changing out the lower limb positioning systems placed on the lower limbs of patients.

Additionally, the lower limb positioning systems may be configured to fix (e.g., hold retain) an angle of a foot of the lower limb of the patient relative to a leg of the lower limb of the patient about an axis of rotation of an ankle that rotatably couples the foot to the leg when the lower limb position systems are placed on the lower limb of the patient (e.g., a position of the foot relative to the leg). When the foot of the patient is fixed at the angle relative to the leg of the patient for a significant amount of time, the patient may lose mobility of the foot relative to the leg (e.g., lose mobility of an ankle of the lower limb). As a result, the patient may need additional physical therapy (e.g., rehab) in order to regain the lost mobility of the foot relative to the leg.

Additionally, when the lower limb positioning systems are placed on the lower limb of the patient, the lower limb positioning systems may trap heat around the lower limb of the patient, which may lead to the lower limb becoming overheated (e.g., a temperature of the lower limb may be greater than a temperature threshold) and/or blood flow through the lower limb being limited (e.g., a flow rate of the blood flow through the lower limb may be below a blood flow rate threshold). For example, if the lower limb positioning system is configured as a boot, the boot may not facilitate optimal ventilation (e.g., air movement, breathing) around the lower limb. The overheating of the lower limb and/or the limiting of blood flow through the lower limb may lead to injuries to the lower limb, which may increase a recovery time of the patient.

Additionally, when the lower limb positioning systems are placed on the lower limb of the patient and the patient is placed in a prone position (e.g., the patient is lying on their stomach), the lower limb positioning system may not be configured to cushion the lower limb of the patient. For example, the lower limb positioning system may include padding configured to cushion the lower limb of the patient when the patient is in a supine position (e.g., the patient is lying on their back), but not when the patient is in the prone position. As a result, the patient may be in discomfort and/or be injured when the lower limb positioning systems are placed on the lower limb of the patient and the patient is placed in the prone position.

Additionally, the lower limb positioning systems may not be configured to gather data associated with the lower limb of the patient. For example, the lower limb positioning systems may not gather data relating to an amount of time that the lower limb of the patient is positioned in a certain position, which could be used to predict a pressure injury to the lower limb of the patient. As a result, the patient may be in discomfort and/or be injured due to the lack of data that is gathered associated with the lower limb of the patient.

Implementations described herein are related to lower limb positioning systems that are less likely to move relative to a lower limb of a patient or a surface where the patient is positioned. Instead, embodiments of the lower limb position systems include various structures (e.g., wraps, straps, high friction surfaces, wings) configured to prevent movement of the lower limb positioning system from an optimal configuration to a non-optimal configuration when the lower limb positioning systems are positioned on the lower limb of the patient. Additionally, embodiments of the lower limb positioning systems include stimulators (e.g., pressure sleeves, mechanical stimulation systems) configured to stimulate the lower limb of the patient when the lower limb positioning systems are positioned on the lower limbs of the patients. As a result of the stimulators stimulating the lower limb of the patient when the lower limb positions systems are positioned on the lower limbs of the patients, a likelihood of pressure injuries developing on the lower limb of the patient may be reduced. In addition, embodiments of the lower limb positioning systems include covers with hard soles that may be placed over boots and/or heel wraps of the lower limb positioning systems. As a result of the covers with the hard soles being placed over the boots and/or heel wraps, patients may ambulate without removing the boots and/or heel wraps, which may easy the process of ambulation for the patient and reduce the risk of the patient falling while ambulating with the lower limb positioning systems installed.

Some embodiments of the lower limb positioning systems include a first portion of the lower limb positioning system that is disposable and a second portion of the lower limb positioning system that is reusable. By only disposing of the first portion and reusing the second portion, an amount of waste generated by the use of the lower limb positioning systems may be reduced. Additionally, some embodiments of the lower limb positioning systems include straps configured to change an orientation of the ankle of the patient relative to the leg of the patient when the lower limb positioning system is installed on the lower limb of the patient. As a result, the orientation of the ankle relative to the leg may be changed without removing the lower limb positioning system and the patient's mobility of the ankle relative to the leg may be maintained. In addition, some embodiments of the lower limb positioning systems include cooling structures (e.g., bladders configured to receive cooling fluids, structures that allow for airflow through the structures) configured to reduce a temperature of the lower limb of the patient when the lower limb positioning system is positioned on the lower limb of the patient. As a result, overheating of the lower limb of the patient and heat related injuries to the lower limb of the patient can be prevented by the cooling element.

Some embodiments of the lower limb positioning systems include a cushion configured to cushion a top side of the lower limb of the patient when the patient is in the prone position and the lower limb positioning systems are positioned on the lower limb of the patient. As a result, pressure injuries to the lower limb of the patient can be prevented when the patient is in the prone position. Additionally, some embodiments of the lower limb positioning systems are configured to gather data associated with the lower limb of the patient when the lower limb positioning systems are installed on the lower limb of the patient. The data may be used to track information related to the lower limb of the patient and may be used to predict an occurrence of a pressure injury to the lower limb of the patient. As a result, the data may be used to take better care of the patient and may prevent injuries to the lower limb pf the patient from developing.

### II. Overview of the Lower Limb Positioning System

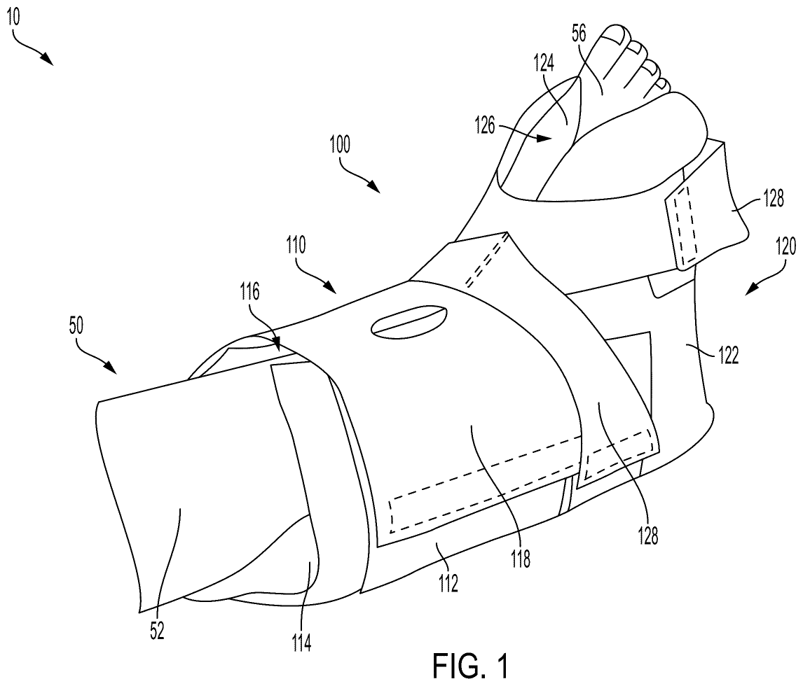

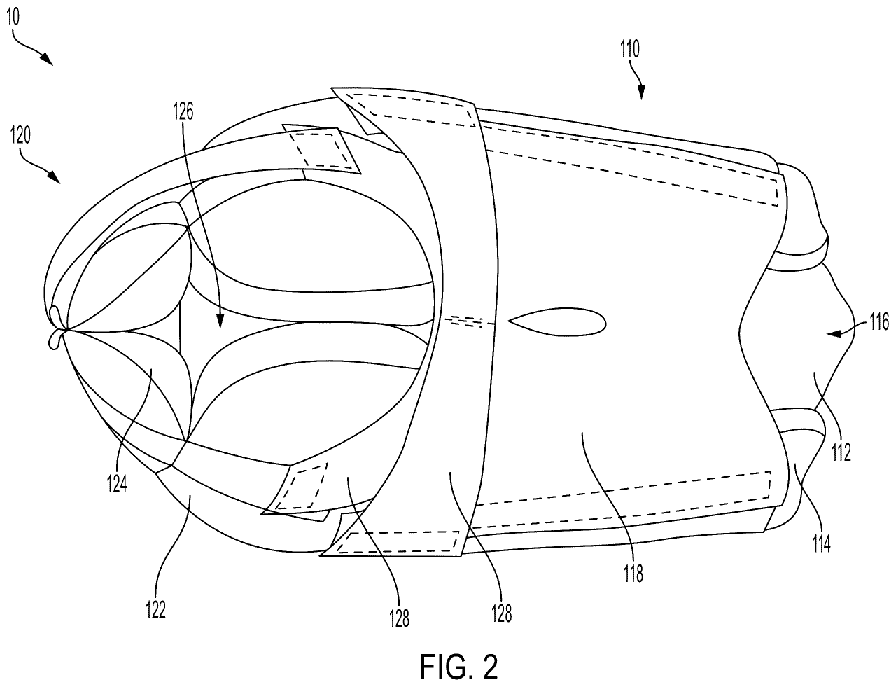

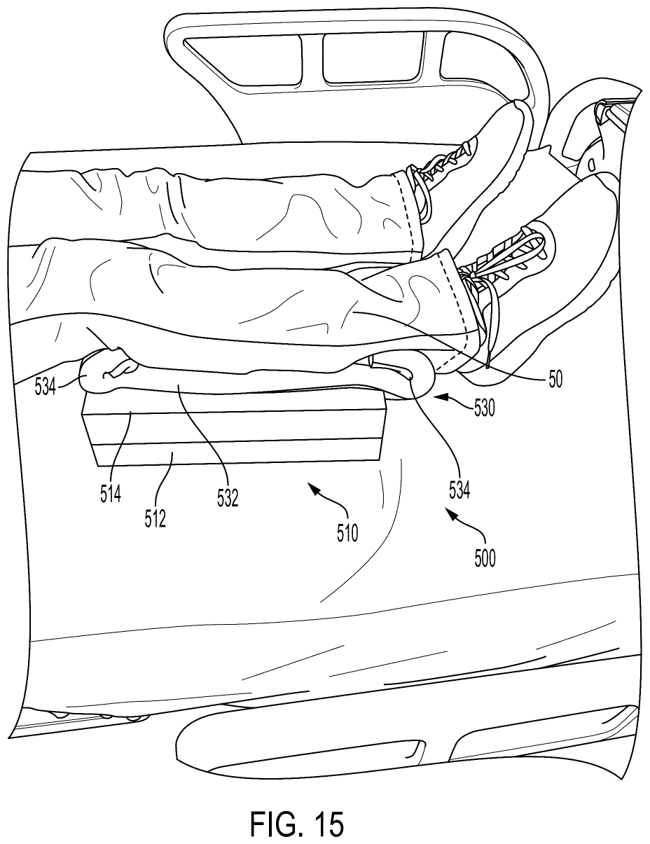

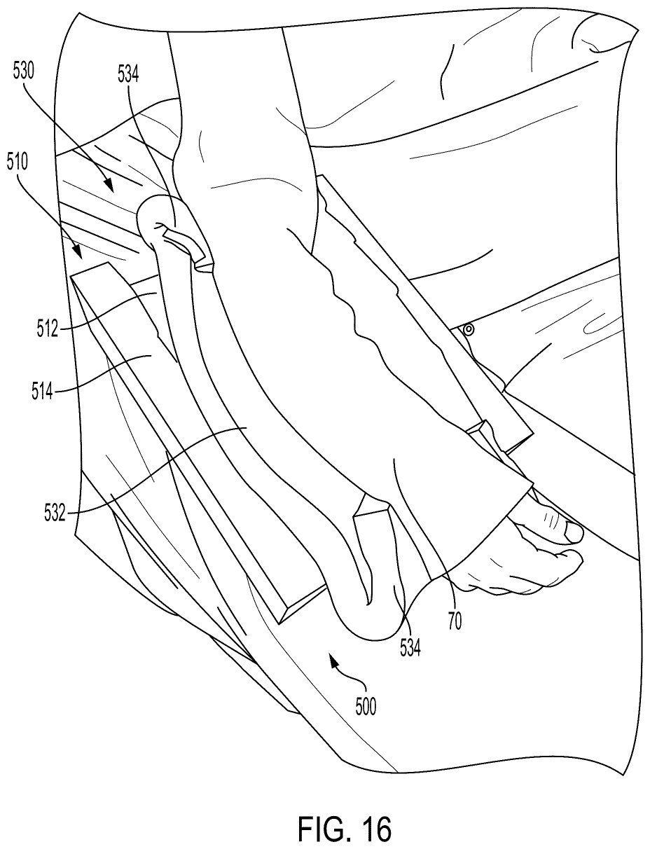

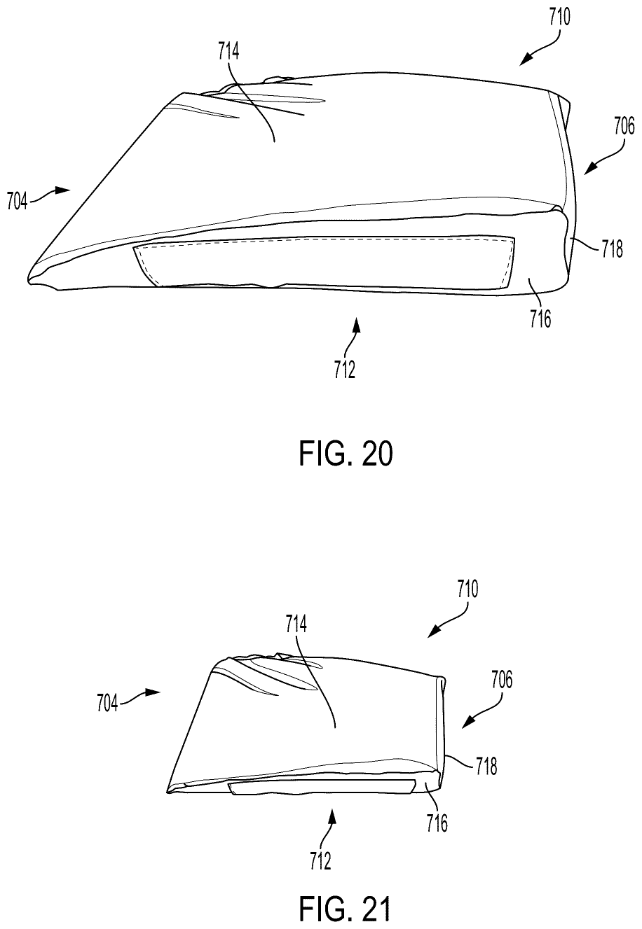

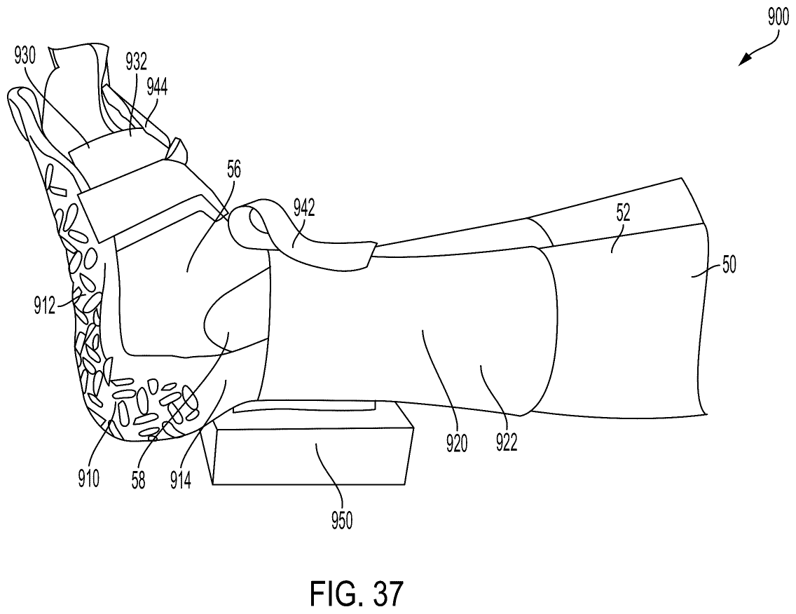

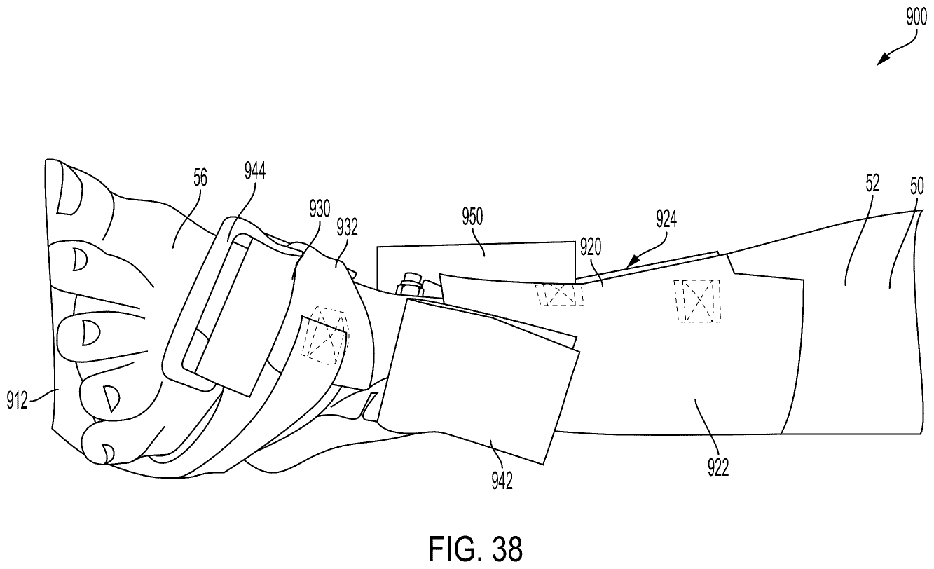

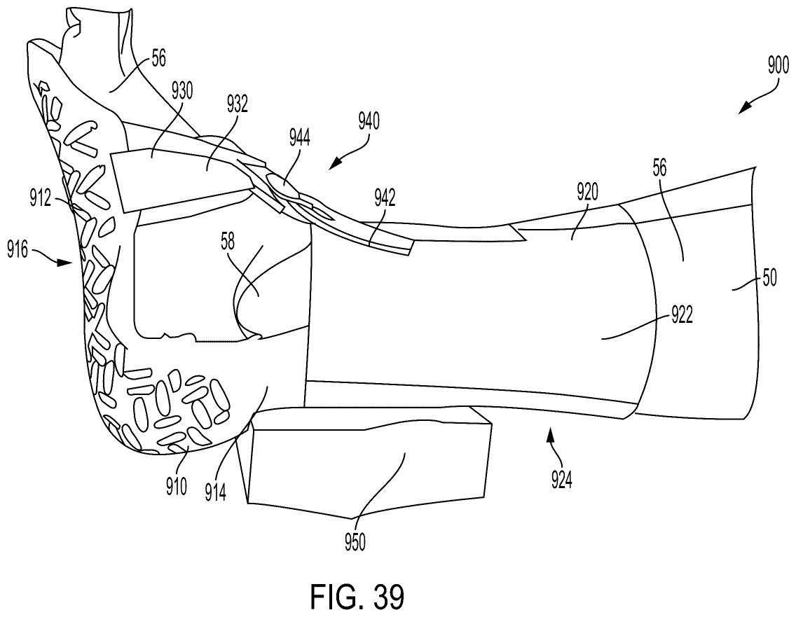

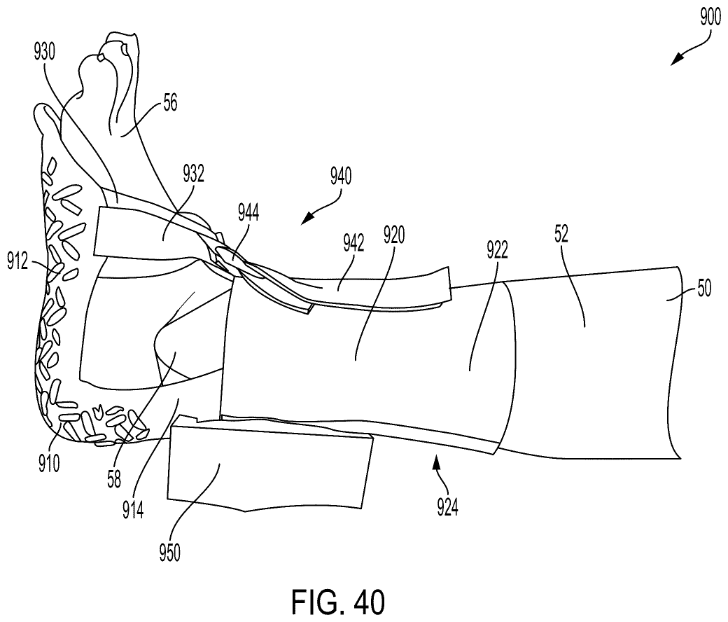

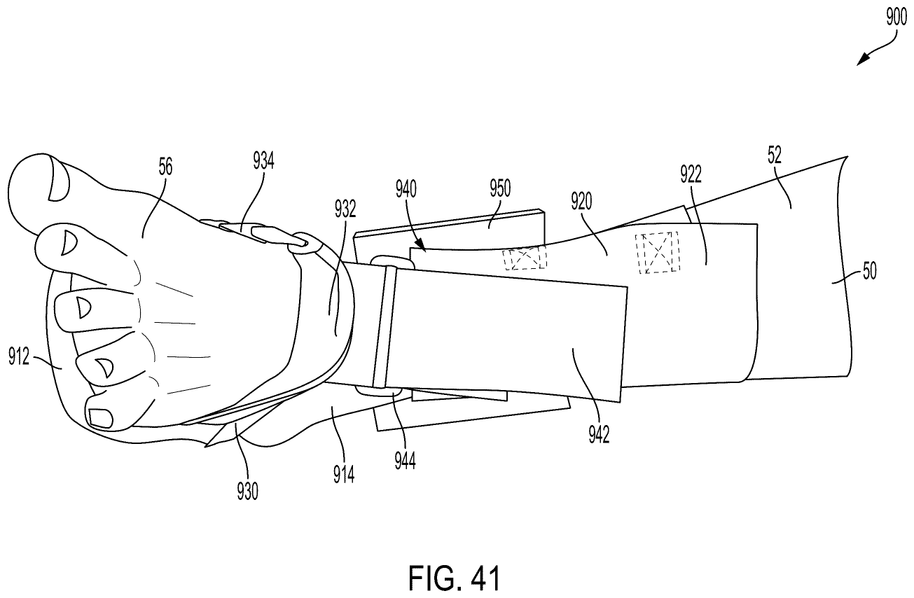

FIGS. 1-47 depict example lower limb positioning systems 10 (e.g., a lower limb protection system, a lower limb pressure injury system). As described in more detail herein, the lower limb positioning systems 10 are configured to be positioning on a lower limb 50 (e.g., a leg) of a patient (e.g., a person, a client). The lower limb 50 of the patient may include a leg portion 52 (e.g., a leg) connected to a torso of the patient. The leg portion 52 includes a shin portion 54 (e.g., a shin) positioned at a lower end of the leg portion 52. the leg portion 52 also includes a foot portion 56 that is rotatably connected to the lower end of the leg portion 52. The foot portion 56 includes a heel portion 58 that is positioned at a back end of the foot portion 56 and extends up toward the leg portion 52. The leg portion 52 also includes an ankle portion 60 that rotatably couples the foot portion 56 to the leg portion 52.

In some embodiments, the lower limb positioning systems 10 positions the lower limb 50 of the patient to protect the lower limb 50 from injury. For example, the lower limb positioning systems 10 may be configured to distribute a force applied on the heel portion 58 from a surface where the patient is positioned to prevent pressure injuries (e.g., bed sores, pressure ulcers) from forming on the heel portion 58 of the lower limb 50. In some embodiments, the lower limb positioning systems 10 is configured to collect data (e.g., via sensors of the lower limb positioning systems 10) associated with the lower limb 50. For example, the lower limb positioning systems 10 may include sensors configured to generate sensor data associated with a pressure applied on the lower limb 50 from a surface when the patient is positioned on the surface or sensor data associated with an amount of time that the lower limb positioning systems 10 are positioned on the lower limb 50 of the patient.

### Overview of the Cushioning Boot

As shown in FIGS. 1-3, the lower limb positioning system 10 includes a cushioning boot 100 (e.g., a lower limb positioning boot, a boot, a padded boot, a pressure off-loading boot) that is configured to be positioned on the lower limb 50 of the patient, according to some embodiments. The cushioning boot 100 includes a leg engagement portion 110 configured to be positioned on the leg portion 52. The cushioning boot 100 also includes a foot engagement portion 120 coupled to the leg engagement portion 110 and configured to be positioned on the foot portion 56 of the lower limb 50.

In some embodiments, the leg engagement portion 110 is configured to be positioned around (e.g., wrapped around) at least a portion of the leg portion 52 of the lower limb 50 to protect the portion of the leg portion 52. In various embodiments, the leg engagement portion 110 is configured to be positioned between the leg portion 52 and the surface supporting the patient to provide a cushion between the leg portion 52 and the surface. In some embodiments, the foot portion 56 is configured to be positioned around at least a portion of the foot portion 56 of the lower limb 50 to protect the portion of the foot portion 56. In various embodiments, the foot engagement portion 120 is configured to be positioned between the foot portion 56 and the surface supporting the patient to provide a cushion between the foot portion 56 and the surface. For example, the foot engagement portion 120 may be configured to be positioned between the heel portion 58 and the surface to prevent pressure injuries from forming on the heel portion 58.

As shown in FIGS. 1-3, the leg engagement portion 110 includes a leg shell 112 configured to be positioned around the portion of the leg portion 52. The leg engagement portion 110 also includes a leg cushion 114 positioned between the leg shell 112 and the leg portion 52. The leg cushion 114 may be coupled (e.g., sewn, adhered) to an inside surface of the leg shell 112. In some embodiments, the leg cushion 114 is configured to be removably coupled to the leg shell 112 to allow for the leg cushion 114 to be cleaned (e.g., washed, sanitized) separately from the leg shell 112. The leg shell 112 and the leg cushion 114 define a leg opening 116 configured to receive the leg portion 52 of the patient when the cushioning boot 100 is positioned on the lower limb 50 of the patient. In some embodiments, as shown in FIGS. 1-3, the leg opening 116 extends through the leg shell 112 and the leg cushion 114 along a length of the leg shell 112 and the leg cushion 114 such that the leg portion 52 of the patient may be placed into the leg opening 116 past the leg shell 112 and the leg cushion 114 when the cushioning boot 100 is positioned on the lower limb 50 of the patient. In other embodiments, the leg shell 112 and the leg cushion 114 define the leg opening 116 inside of and along the length of the leg shell 112 and the leg cushion 114 such that the lower limb 50 of the patient is slid through the leg opening 116 along the length of the leg shell 112 and the leg cushion 114 when the cushioning boot 100 is positioned on the lower limb 50 of the patient.

As shown in FIGS. 1-3, the leg engagement portion 110 includes a leg closure panel 118 configured to couple the leg engagement portion 110 to the leg portion 52. For example, the leg closure panel 118 may be a strap configured to couple the leg engagement portion 110 to the leg portion 52. In some embodiments, the leg closure panel 118 is coupled to the leg shell 112 at a first location on a first side of the leg opening 116 (e.g., when the leg opening 116 extends through the leg shell 112 and the leg cushion 114 along the length of the leg shell 112 and the leg cushion 114) and is configured to be removably coupled (e.g., via a hook and loop style coupling, via an adhesive) to the leg shell 112 at a second location on a second side of the leg opening 116 such that the leg closure panel 118 covers the leg opening 116 when the leg closure panel 118 is coupled to the leg shell 112 at the second location to prevent the leg portion 52 from being removed from the leg engagement portion 110. In various embodiments, the leg closure panel 118 is configured to cinch the leg shell 112 and the leg cushion 114 around the leg portion 52 to prevent the leg portion 52 from being removed from the leg engagement portion 110.

As shown in FIGS. 1-3, the foot engagement portion 120 includes a foot shell 122 configured to be positioned around the portion of the foot portion 56 and a foot cushion 124 configured to be positioned between the foot shell 122 and the foot portion 56. In some embodiments, the foot shell 122 is coupled to the leg shell 112 and/or the foot cushion 124 is coupled to the foot cushion 124. For example, the foot cushion 124 and the leg cushion 114 may be coupled proximate the heel portion 58 of the lower limb 50 to provide cushioning at the heel portion 58. In other embodiments, the foot shell 122 and the leg shell 112 and/or the foot cushion 124 and the leg cushion 114 are integrally formed (e.g., formed out of a single unitary body). For example, the foot shell 122 and the leg shell 112 may be formed from a single piece of fabric.

The foot cushion 124 may be coupled to an inside surface of the foot shell 122. In some embodiments, the foot cushion 124 is configured to be removably coupled to the foot shell 122 to allow for the foot cushion 124 to be cleaned separately from the foot shell 122. The foot shell 122 and the foot cushion 124 define a foot opening 126 configured to receive the foot portion 56 of the patient when the cushioning boot 100 is positioned on the lower limb 50 of the patient. For example, the foot cushion 124 may be removably coupled to the foot shell 122 using hook and loop fasteners (e.g., Velcro), snaps, clasps, magnets, or other similar structures. In some embodiments, as shown in FIGS. 1-3, the foot opening 126 extends through the foot shell 122 and the foot cushion 124 along a length of the foot shell 122 and the foot cushion 124 such that the foot portion 56 of the patient may be placed into the foot opening 126 past the foot shell 122 and the foot cushion 124 when the cushioning boot 100 is positioned on the lower limb 50 of the patient. In other embodiments, the foot shell 122 and the foot cushion 124 define the foot opening 126 inside of and along the length of the foot shell 122 and the foot cushion 124 such that the lower limb 50 of the patient is slid through the foot opening 126 along the length of the foot shell 122 and the foot cushion 124 when the cushioning boot 100 is positioned on the lower limb 50 of the patient.

As shown in FIGS. 1-3, the foot engagement portion 120 includes a foot closure panel 128 configured to couple the foot engagement portion 120 to the foot portion 56. For example, the foot closure panel 128 may be a strap configured to couple the foot engagement portion 120 to the foot portion 56. In some embodiments, the foot closure panel 128 is coupled to the foot shell 122 at a first location on a first side of the foot opening 126 (e.g., when the foot opening 126 extends through the foot shell 122 and the foot cushion 124 along the length of the foot shell 122 and the foot cushion 124) and is configured to be removably coupled to the foot shell 122 at a second location on a second side of the foot opening 126 such that the foot closure panel 128 covers the foot opening 126 when the foot closure panel 128 is coupled to the foot shell 122 at the second location to prevent the foot portion 56 from being removed from the foot engagement portion 120. In various embodiments, the foot closure panel 128 is configured to cinch the foot shell 122 and the foot cushion 124 around the foot portion 56 to prevent the foot portion 56 from being removed from the foot engagement portion 120. In some embodiments, the foot engagement portion 120 includes a first of the foot closure panels 128 coupled to the foot shell 122 at the first location and configured to be removable coupled to the foot shell 122 at the second location and a second of the foot closure panels 128 coupled to the foot shell 122 at the first location and configured to be removable coupled to the leg shell 112 at the second location to further secure the lower limb 50 in the cushioning boot 100. In other embodiments, the foot engagement portion 120 includes multiple of the foot closure panels 128 coupled between difference locations of the foot shell 122 to secure the lower limb in the cushioning boot 100.

In some embodiments, the cushioning boot 100 is formed and/or coated with materials that are easy to clean (e.g., materials that are compatible with cleaning products, materials that can be run through a laundry machine). For example, the cushioning boot 100 may be formed and/or coated with silicone, nylon, polyester, or other materials that are easy to sterilize (e.g., chemically sterilize, sterilize using high temperatures), chemically resistant, and/or able to corrosion resistant. In some embodiments, the cushioning boot 100 is formed and/or coated with materials that have antiseptic properties (e.g., materials that kill pathogens on contact, materials that are not conducing to hosting pathogens). For example, the cushioning boot 100 may be formed and/or coated with silver nanoparticles, graphene oxide, or other materials that are detrimental to pathogens. In some embodiments, the cushioning boot 100 is formed and/or coated with materials with a high durability (e.g., materials that can retain desired properties through multiple uses). For example, the cushioning boot 100 may be formed and/or coated with polyester, Kevlar, nylon, or other materials that can withstand repeated uses. In some embodiments, the leg cushion 114 and/or the foot cushion 124 are formed out of a cushioning material configured to distribute pressure around the leg portion 52 and/or the foot portion 56 of the lower limb 50 of the patient. For example, the leg cushion 114 and/or the foot cushion 124 may be formed out of polyurethane foam, polyethylene foam, silicone foam, or other materials that provide cushioning and support. As another example, the leg cushion 114 and/or the foot cushion 124 may be formed out of a pillow-like material configured to replicate the feel of typical hospital pillows which are regularly used for supporting the lower limbs 50 of patients.

In some embodiments, the leg shell 112 and/or the foot shell 122 is formed and/or coated with a material with a high coefficient of friction (e.g., higher than the surface under the patient). For example, the leg shell 112 and/or the foot shell 122 may be formed and/or coated with silicone rubber, neoprene, polyurethane foam, or other materials with coefficients of friction that are higher than standard bed sheets on a hospital bed. When the patient is positioned on the surface and the cushioning boot 100 is positioned on the lower limb 50 of the patient, the high coefficient of friction of the leg shell 112 and/or the foot shell 122 may reduce the possibility of the cushioning boot 100 moving relative to the surface.

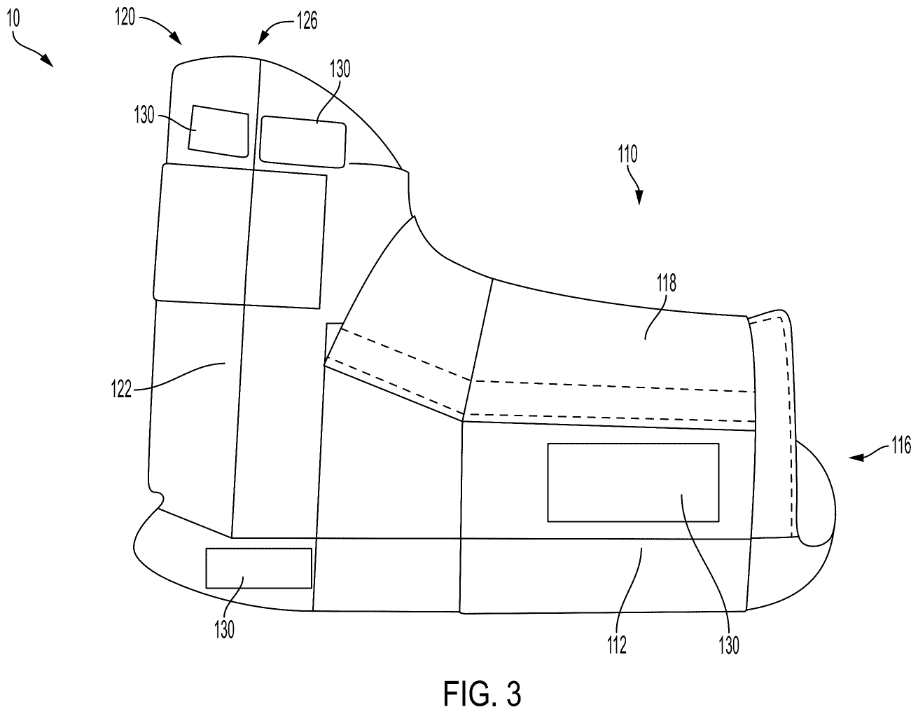

As shown in FIG. 3, the cushioning boot 100 includes pockets 130 configured to receive components of the lower limb positioning system 10. For example, the pockets 130 may be configured to receive sensors of the lower limb positioning system 10, inflation devices of the lower limb positioning system 10, or extra cushions of the lower limb positioning system 10. The pockets 130 may be positioned on the leg engagement portion 110 and/or the foot engagement portion 120 of the cushioning boot 100. For example, a first of the pockets 130 may be positioned on the foot engagement portion 120 of the cushioning boot 100 proximate a position of the heel portion 58 of the lower limb 50 when the cushioning boot 100 is positioned on the lower limb 50. The first of the pockets 130 may be configured to receive a pressure sensor configured to generate sensor data associated with a pressure on the heel portion 58 of the lower limb 50 when the cushioning boot 100 is positioned on the lower limb 50. As another example, a second of the pockets 130 may be positioned on the leg engagement portion 110 of the cushioning boot 100. The second of the pockets 130 may be configured to receive a mechanical stimulation device configured to stimulate the lower limb 50 when the cushioning boot 100 is positioned on the lower limb 50. In various embodiments, the pockets 130 may include closure fasteners configured to close openings of the pockets 130. For example, the pockets 130 may include zippers, buttons, or other means configured to close the openings of the pockets 130 and prevent access into the openings of the pockets 130.

The lower limb positioning system 10 may include any of the embodiments disclosed or described in U.S. Pat. No. 7,798,984 entitled “HEEL ULCER PREVENTION AND CUSHIONING BOOT” filed Sep. 30, 2005, which is hereby incorporated by reference herein in its entirety.

### Overview of Mechanical Stimulation System

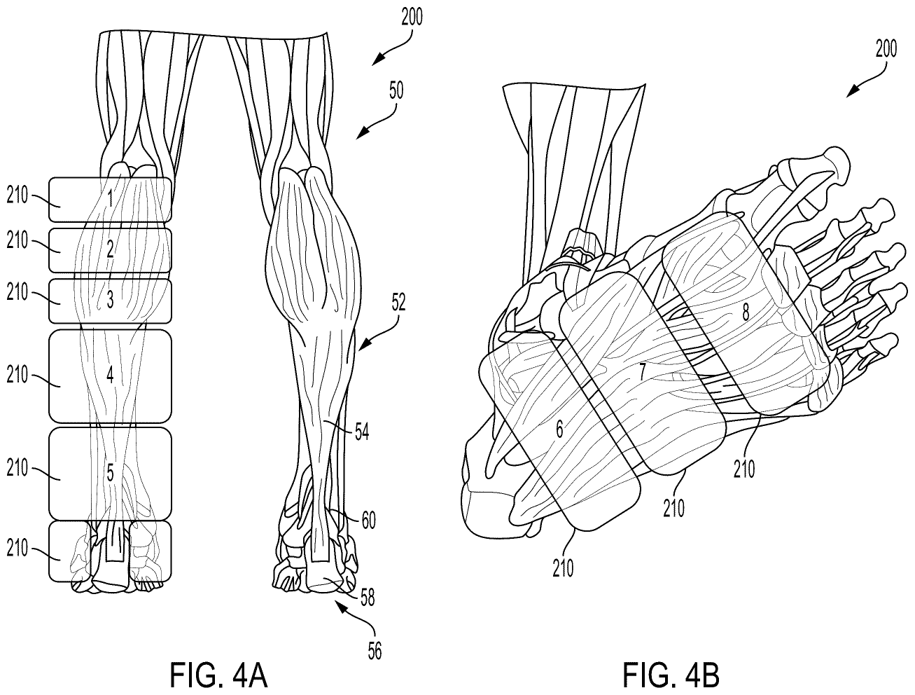

As shown in FIGS. 4A and 4B, the lower limb positioning system 10 includes a mechanical stimulation system 200 (e.g., a vibration system, a vibration stimulation system) configured to stimulate the lower limb 50 of the patient, according to some embodiments. By stimulating the lower limb 50 of the patient via mechanical stimulations (e.g., vibrations), the mechanical stimulation system 200 may mitigate or treat pressure injuries to the lower limb 50. The mechanical stimulation system 200 may increase blood and lymphatic circulation throughout the lower limb 50, which may accelerate healing of the lower limb 50 and decrease a length of a hospital stay required by the patient.

As shown in FIGS. 4A and 4B, the mechanical stimulation system 200 includes mechanical stimulators 210 positioned proximate locations of the lower limb 50 of the patient. Each of the mechanical stimulators 210 may be operated to provide mechanical stimulation to the locations of the lower limb 50 proximate the mechanical stimulators 210. For example, each of the mechanical stimulators 210 may include a vibration actuator or a vibration motor configured to be operated to generate vibration and a stimulation plate configured to transfer the vibration generated by the vibration actuators or the vibration motors to the lower limb 50. The vibration generated by the mechanical stimulators 210 may be transferred to the lower limb 50 through the stimulation plates as the mechanical stimulation. In some embodiments, the mechanical stimulators 210 are positioned proximate muscle groups of the lower limb 50 in order to provide the mechanical stimulation to the muscle groups. For example, the mechanical stimulation system 200 may include nine of the mechanical stimulators 210, each of which is positioned proximate nine of the muscle groups of the lower limb 50 in order to provide the mechanical stimulation to the nine of the muscle groups of the lower limb 50.

In some embodiments, the mechanical stimulators 210 are received by the pockets 130 of the cushioning boot 100 to position the mechanical stimulators 210 proximate the lower limb 50 when the cushioning boot 100 is positioned on the lower limb 50. For example, the pockets 130 may be positioned on the leg engagement portion 110 and the foot engagement portion 120 such that the mechanical stimulators 210 are positioned proximate the muscle groups of the lower limb 50 when the mechanical stimulators 210 are received by the pockets 130 and the cushioning boot 100 is positioned on the lower limb 50. In other embodiments, the mechanical stimulators 210 are positioned on other components (e.g., foot wraps, heel protectors) of the lower limb positioning system 10 to position the mechanical stimulators 210 proximate the lower limb 50 or the mechanical stimulation system 200 may be positioned on the lower limb 50 separate from the other components of the lower limb positioning system 10.

In some embodiments, the mechanical stimulators 210 provide the mechanical stimulation to the lower limb 50 at a frequency of 40-50 Hertz (Hz), or approximately 47 Hz, and with an amplitude of 20-30 micron, or approximately 25 micron. These frequencies and amplitudes have been shown in clinical research to increase blood and lymphatic circulation. In other embodiments, the mechanical stimulators 210 provide the mechanical stimulation to the lower limb 50 at other frequencies (e.g., lower than 40 Hz, higher than 50 Hz) and/or other amplitudes (e.g., less than 20 microns, greater than 30 microns). In some embodiments, each of the mechanical stimulators 210 are operated in a sequential pattern to encourage blood flow through the lower limb 50. For example, the mechanical stimulators 210 may be operated in a first sequential pattern in a direction downward along a length of the lower limb 50 of the patient. The mechanical stimulators 210 may be operated in an order from a first of the mechanical stimulators 210 positioned closest to a proximal end of the lower limb 50 (e.g., a top of the leg portion 52, a back of the foot portion 56) to a last of the mechanical stimulators 210 positioned closest to a distal end of the lower limb 50 (e.g., a bottom of the leg portion 52, a front of the foot portion 56) to encourage blood flow from the proximal end of the lower limb 50 towards the distal end of the lower limb 50. As another example, the mechanical stimulators 210 may be operated in a second sequential pattern in a direction upward along the length of the lower limb 50 of the patient. The mechanical stimulators 210 may be operated in an order from a first of the mechanical stimulators 210 positioned closest to the distal end of the lower limb 50 to a last of the mechanical stimulators 210 positioned closest to the proximal end of the lower limb 50 to encourage blood flow from the distal end of the lower limb 50 toward the proximal end of the lower limb 50.

### Overview of Compression System

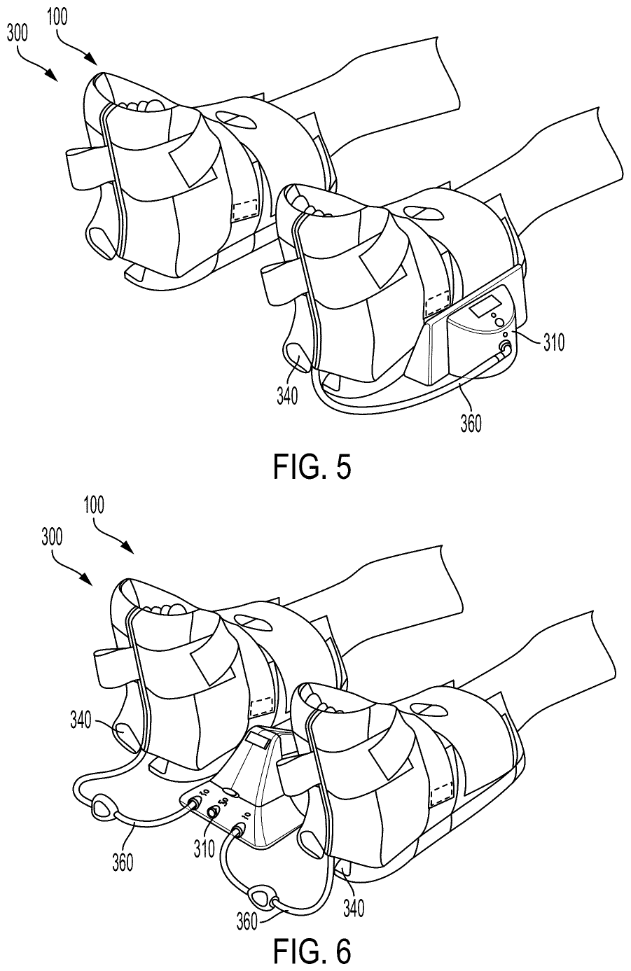

As shown in FIGS. 5-9, the lower limb positioning system 10 includes a compression system 300 (e.g., an inflation system) configured to provide compression to the lower limb 50 of the patient, according to some embodiments. By providing compression to the lower limb 50 of the patient, the compression system 300 may prevent, minimize, or improve deep vein thrombosis (e.g., blood clots) in the lower limb 50 of the patient while also preventing, minimizing, or improving pressure injuries to the lower limb 50 of the patient. The compression may assist veins in the lower limb 50 of the patient with returning blood flow toward the torso of the patient and reduce blood stasis in the lower limb 50 of the patient that may occur when the lower limb 50 of the patient is immobile for an extended period of time (e.g., when the lower limb positioning system 10 is positioned on the lower limb 50 to immobilized the lower limb 50).

As shown in FIGS. 5-9 the compression system 300 includes a compressor system 310 (e.g., compression device, compressor system, pump assembly) configured to operate the compression system 300 and compression cells 340 (e.g., compression elements, compression sleeves, compression wraps) configured to provide pressure to the lower limb 50. In some embodiments, the compression cells 340 are bladders (e.g., flexible vessels) positioned proximate the lower limb 50 that provide the compression to the lower limb 50 when the bladders are inflated. For example, the compression cells 340 may be formed of plastic or other flexible materials. In other embodiments, the compression cells 340 are other compression sleeves configured to provide the compression to the lower limb 50 (e.g., mechanically operated compression sleeves, electrically operated compression sleeves).

In some embodiments, the compression system 300 includes a single of the compression cells 340 configured to provide the compression to the lower limb 50. For example, the single of the compression cells 340 may be provided around the lower limb 50 and configured to provide the compression to the lower limb 50. In other embodiments, the compression system 300 includes multiple of the compression cells 340 configured to provide the compression to the lower limb 50. For example, a first of the compression cells 340 may be positioned on a first side of the lower limb 50 and a second of the compression cells 340 may be positioned on a second side of the lower limb 50 opposing the first side of the lower limb 50. The first of the compression cells 340 and the second of the compression cells 340 may compress the lower limb 50 between the first of the compression cells 340 and the second of the compression cells 340.

The compressor system 310 is configured to operate the compression cells 340 to provide the pressure to the lower limb 50. In some embodiments, the compressor system 310 provides a pressurized fluid (e.g., a water, air) to the compression cells 340 to inflate the compression cells 340 to provide the compression to the lower limb 50 (e.g., when the compression cells 340 are bladders). The compressor system 310 may be fluidly coupled to the compression cells 340 by compression hoses 360 (e.g., compression conduits, compression tubes). In other embodiments, the compressor system 310 operates the compression cells 340 to provide the compression to the lower limb 50 using other means (e.g., providing electrical power to the compression cells 340, providing control signals to the compression cells 340).

As shown in FIG. 5, the compression system 300 is configured as a single limb compression system configured to provide compression to one of the lower limbs 50, according to some embodiments. The compressor system 310 may be configured to operate the compression cells 340 proximate the one of the lower limbs 50 to provide the compression to the one of the lower limbs 50. In some embodiments, the compressor system 310 is coupled to the one of the lower limbs 50 when the compression system 300 is configured as the single limb compression system. For example, the compressor system 310 may be coupled to at least one of the compression cells 340 proximate the one of the lower limbs 50.

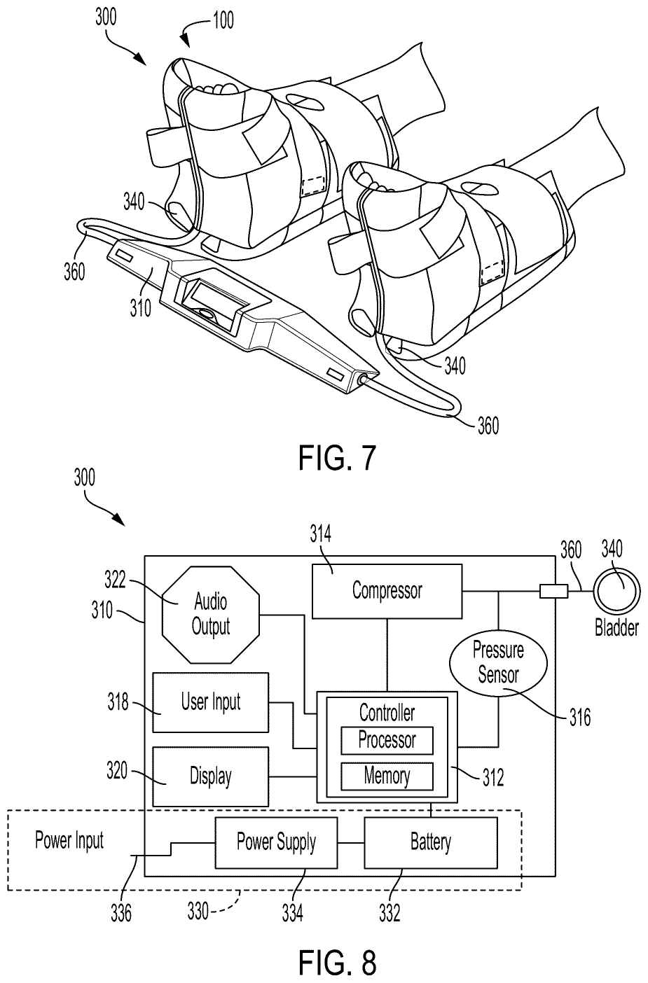

As shown in FIGS. 6 and 7, the compression system 300 is configured as a multiple limb compression system configured to provide compression to a plurality of the lower limbs 50, according to some embodiments. The compressor system 310 may be configured to operate the compression cells 340 proximate each of the lower limbs 50 (e.g., a first lower limb and a second lower limb) of the patient to provide the compression to each of the lower limbs 50. In some embodiments, the compressor system 310 is positioned between the lower limbs 50 when the compression system 300 is configured as the multiple limb compression system. In other embodiments, the compressor system 310 is coupled to at least one of the lower limbs 50 when the compressor system 310 is configured as the multiple limb compression system. For example, the compressor system 310 may be coupled to a first of the lower limbs 50, a second of the lower limbs 50, or coupled between the first of the lower limbs 50 and the second of the lower limbs 50.

As shown in FIGS. 5-7, the compression system 300 is integrated into the cushioning boot 100 to provide compression to the lower limb 50 of the patient when the cushioning boot 100 is provided on the lower limb 50, according to some embodiments. For example, the compression cells 340 may be received by the pockets 130 of the cushioning boot 100 to position the compression cells 340 proximate the lower limb 50 when the cushioning boot 100 is positioned on the lower limb 50. As another example, the compression cells 340 may be positioned between (i) the foot shell 122 and/or the foot cushion 124 and (ii) the leg shell 112 and the leg cushion 114, and/or inside of the foot opening 126 and/or the leg opening 116 to position the compression cells 340 proximate the lower limb 50 when the cushioning boot 100 is positioned on the lower limb 50. In various embodiments, the compression cells 340 are positioned on other elements (e.g., foot wraps, heel protectors) of the lower limb positioning system 10 to position the compression cells 340 proximate the lower limb 50 or the compression system 300 may be positioned on the lower limb 50 separate from the other components of the lower limb positioning system 10.

As shown in FIG. 9, the compression cell 340 includes a heel compression portion 342 and compression wings 344 extending from the heel compression portion 342. A first of the compression wings 344 may extend from the heel compression portion 342 in a first direction and a second of the compression wings 344 may extend from the heel compression portion 342 in a second opposing direction. The compression wings 344 may be moved (e.g., pivoted) between a lowered position where the compression wings 344 are generally parallel with the heel compression portion 342 and a raised position where the compression wings 344 are generally non-parallel (e.g., perpendicular) with the heel compression portion 342. When the compression cell 340 is positioned on the lower limb 50 of the patient, the heel compression portion 342 may be positioned under the heel portion 58 of the lower limb 50 and the compression wings 344 may be positioned proximate the shin portion 54 of the lower limb 50. In some embodiments, the heel compression portion 342 may include more padding (e.g., cushioning) than the compression wings 344 to provide additional cushioning to the heel portion 58 of the lower limb 50. In some embodiments, the heel compression portion 342 are configured to expand to a greater thickness than the compression wings 344 when pressurized by the compressor system 310. For example, the heel compression portion 342 may be configured to expand to a thickness that is twice as thick as the compression wings 344 such that the compression cell 340 applies a higher pressure on the heel portion 58 of the lower limb 50 than on the shin portion 54 of the lower limb 50. By applying the higher pressure on the heel portion 58 of the lower limb 50, pressure injuries to the heel portion 58 may be avoided.

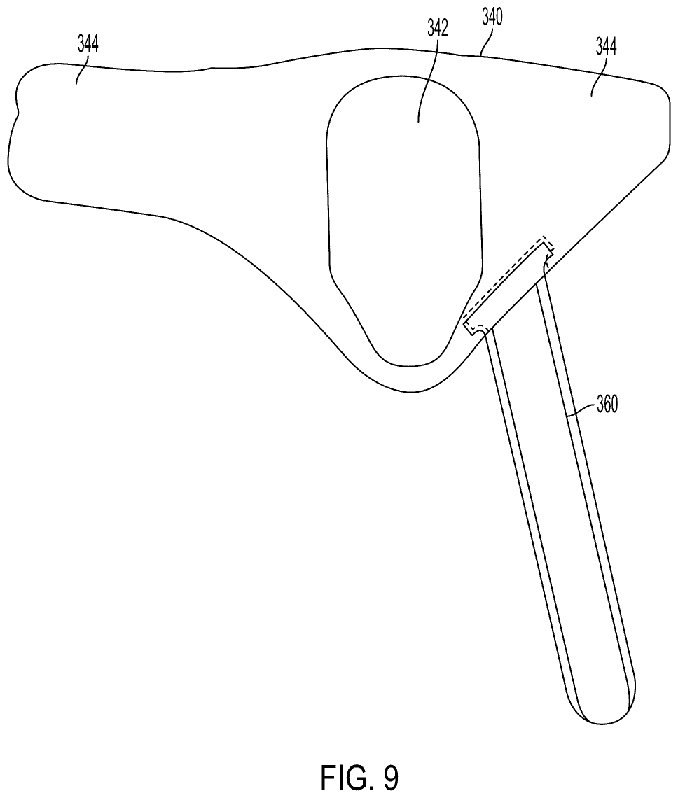

As shown in FIG. 8, the compressor system 310 includes a controller 312 configured to control the operation of the compression system 300, according to some embodiments. The controller 312 may include a processor and memory that may store instructions that, when executed by the processor, cause the controller 312 to perform various processes described herein. In other embodiments, the compressor system 310 does not include the controller 312 and is operated by a controller of the lower limb positioning system 10.

As shown in FIG. 8, the compressor system 310 includes a compressor 314 configured to operate the compression cells 340 to provide the pressure to the lower limb 50 (e.g., when the compression cells 340 are configured to be inflated to provide the pressure to the lower limb 50), according to some embodiments. The controller 312 may operate the compressor 314 to alternatively provide a pressurized fluid to the compression cells 340 and remove the pressurized fluid from the compression cells 340 (e.g., through the compression hoses 360). For example, the controller 312 may operate the compressor 314 with a frequency for the compression cells 340 to apply the pressure to the lower limb 50 with the frequency. In some embodiments, the compressor 314 is configured to auto-deflate the compression cells 340 when the pressure of the pressurized fluid supplied to the compression cells 340 by the compressor 314 reaches a target pressure.

In some embodiments, the compressor 314 is sized to be approximately 50.8 millimeters (mm)×101.6 mm×25.4 mm (e.g., 2″×4″×1″). In some embodiments, the controller 312 operates the compressor 314 to provide the pressurized fluid to the compression cells 340 in an inflation time period that ranges from one second to thirty seconds, maintain the pressurized fluid in the compression cells 340 for a pressurized time period that ranges from under one second to ninety seconds, and then remove the pressurized fluid from the compression cells 340 for a deflation time period that ranges from one second to thirty seconds. In some embodiments, the controller 312 operates the compressor 314 to provide the pressurized fluid with a pressure in a range from 50 to 400 millimeters of mercury (mmHg).

In some embodiments, the controller 312 operates the compression system 300 such that each of the compression cells 340 are operated in a sequential pattern to encourage blood flow through the lower limb. For example, the controller 312 may operate the compressor 314 to provide the pressurized fluid to the compression cells 340 in a sequential pattern from a first of the compression cells 340 positioned closest to a proximal end of the lower limb 50 to a last of the compression cells 340 positioned closest to a distal end of the lower limb 50 to encourage blood flow from the proximal end of the lower limb 50 towards the distal end of the lower limb 50. As another example, the controller 312 may operate the compressor 314 to remove the pressurized fluid from the compression cells 340 in a sequential pattern from a first of the compression cells 340 positioned closest to the distal end of the lower limb 50 to a last of the compression cells 340 positioned closest to the proximal end of the lower limb 50 to encourage blood flow from the distal end of the lower limb 50 toward the proximal end of the lower limb 50.

As shown in FIG. 8, the compressor system 310 includes a sensor 316 (e.g., compression sensor, a pressure sensor) configured to generate sensor data associated with the operation of the compression cells 340 and/or the compressor 314, according to some embodiments. For example, the sensor 316 may generate sensor data associated with the pressure of the pressurized liquid provide by the compressor 314 to the compression cells 340. As another example, the sensor 316 may generate sensor data associated with the pressure applied on the lower limb 50 by the compression cells 340.

In some embodiments, the sensor 316 is configured to provide the sensor data to the controller 312. The controller 312 may operate the compressor 314 based on the sensor data received from the sensors 316. For example, while operating the compressor 314 to provide the pressurized fluid to the compression cells 340, the controller 312 may continue to operate the compressor 314 until receiving sensor data from the sensor 316 indicating that the pressure applied by the compression cell 340 has reached an upper pressure threshold. As another example, while operating the compressor 314 to remove the pressurized fluid to the compression cells 340, the controller 312 may continue to operate the compressor 314 until receiving sensor data from the sensor 316 indicating that the pressure applied by the compression cells 340 has reached a lower pressure threshold.

As shown in FIG. 8, the compressor system 310 includes a user interface 318 (e.g., a graphical user interface, a physical user interface) configured receive user inputs from an operator of the compression system 300 associated with the operation of the compression system 300, according to some embodiments. The user interface 318 may provide input data to the controller 312 based on the user inputs received from the operator. For example, the user interface 318 may include a knob associated with the pressure applied by the compression cells 340 on the lower limb 50. The knob may be configured to be rotated by the operator and the user interface 318 may generate input data based on the rotation of the know. The user interface 318 may provide the input data to the controller 312 and the controller 312 may operate the compressor 314 based on the input data. The user interface 318 may allow for the operator of the compression system 300 to change an amount of pressure applied by the compression system 300, a frequency that the compression system 300 applies the pressure, a sequence of the pressure applied by the compression system 300, or other variables associated with the operation of the compression system 300. In some embodiments, the user interface 318 may be provided by a user device (e.g., a cell phone, a computer) configured to receive the user inputs from the operator and provide input data to the controller 312. For example, the user interface 318 may be provided by a cell phone configured to wirelessly (e.g., via a network, via Bluetooth) provide the input data to the controller 312.

As shown in FIG. 8, the compressor system 310 includes a display 320 configured to provide a visual output associated with the compression system 300 and a speaker 322 configured to provide an audio output associated with the compression system 300, according to some embodiments. The controller 312 may operate the display 320 and/or the speaker 322 based on the operation of the compression system 300. For example, the display 320 may provide an interface (e.g., a graphical user interface) that includes an indication of the pressure applied by the compression cells 340 on the lower limb 50. As another example, the speaker 322 may provide an audio alert when the pressure applied by the compression cell 340 on the lower limb 50 exceeds the upper pressure threshold so that the operator of the compression system 300 can modify the operation of the compression system 300 to prevent injury to the lower limb 50. In various embodiments (e.g., when the compressor system 310 does not include the display 320, in addition to the display 320, the compressor system 310 includes lights (e.g., LED lights) configured to provide lights indicating a status of the compression system 300. For example, a green light may indicate that the compression cell 340 is being pressurized by the compressor 314, a yellow light may indicate that the compression cell 340 is being held at a pressure, and a red light may indicate that the compression cell 340 is being depressurized. In some embodiments, the user interface 318 is associated with the display 320 and/or the speaker 322. For example, the display 320 may be configured as a touch screen that displays at least a portion of the user interface 318.

As shown in FIG. 8, the compressor system 310 includes a rechargeable power system 330 (e.g., a battery power system) configured to provide power (e.g., electrical power) to the components of the compression system 300 (e.g., the controller 312, the compressor 314, the sensor 316), according to some embodiments. The rechargeable power system 330 may include a battery 332 electrically coupled to the components of the compression system 300 and configured to store electrical power and provide the electrical power to the components of the compression system 300, a power supply 334 (e.g., a converter) electrically coupled to the battery 332, and a power input 336 electrically coupled to the power supply 334 and configured to receive power from an external power system (e.g., from a wall socket). In some embodiments, the power supply 334 is configured to convert the power received from the power input 336 before providing the power to the battery 332 (e.g., from AC to DC, from DC to AC). By including the rechargeable power system 330, the compressor system 310 may continue to operate the compression cells 340 when the power input 336 is disconnected from the external power supply. In other embodiments, the compressor system 310 includes a continuous power system configured to receive a constant flow of power from the external system to provide power to the components of the compression system 300.

### Overview of Hard-Soled Boot Cover

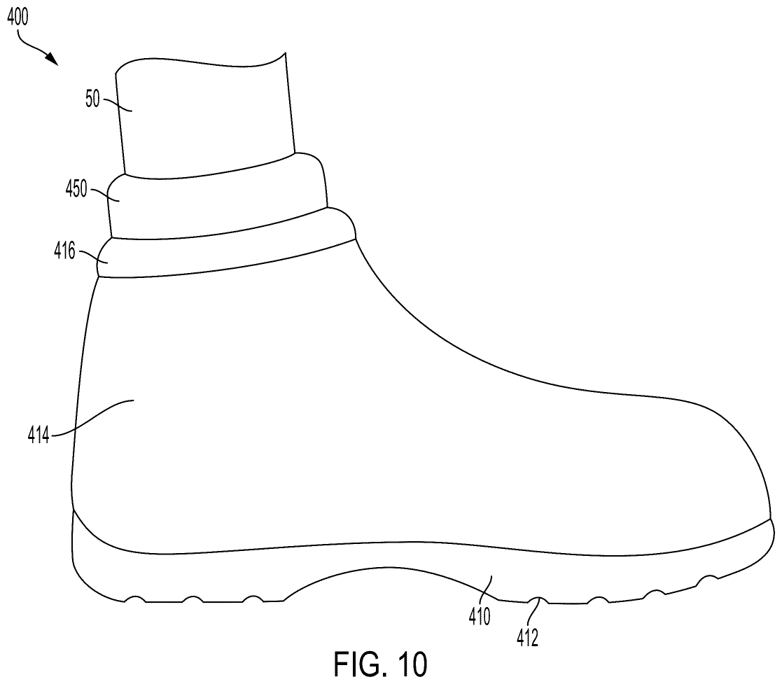

As shown in FIG. 10, the lower limb positioning system 10 includes a boot cover 400 (e.g., a hard soled boot cover, a heel wrap cover) configured to be used with another element of the lower limb positioning system 10 (e.g., a boot, a heel cover, a heel protector). Other configurations of heel protectors (e.g., protection boots, protection wraps) include a soft bottom that can cause imbalance for patients wearing the heel protectors or a lack of traction between the heel protectors and the ground. As a result of the soft bottom, patients may not be able to ambulate (e.g., walk) while wearing the heel protectors. In order to ambulate, the patients may need to remove the heel protectors, which may take additional time or manpower to remove, the patient may choose to attempt to ambulate in the heel protectors which could make the patient a fall risk, or the patient may choose to avoid ambulation entirely, which could slow the recovery of the patient. The boot cover 400 is configured to easily slide over a heel protector 450 positioned on the lower limb 50 to allow for the patient to ambulate without removing the heel protector 450. Additionally, the boot cover 400 may be easily removed before the patient returns to bed, which may prevent materials (e.g., dirt, pathogens) that are picked up by the boot cover 400 during ambulation from reaching the bed. In some embodiments, the boot cover 400 is configured to be a single-patient-use product that may be reusable by the same patient or disposable after one or more uses by the patient.

As shown in FIG. 10, the boot cover 400 includes a sole 410 (e.g., a rigid sole, a rubber sole, a hard sole, an integrated external rigid sole) configured to contact the ground while the boot cover 400 is positioned on the lower limb 50 of the patient. The sole 410 is configured to be rigid to provide structure between the lower limb 50 of the patient and the ground while the patient is ambulating. For example, the sole 410 may be configured to resist deformation along a length of the sole 410 to provide a sturdy surface for the patient during ambulation. When the boot cover 400 is positioned on the on the lower limb 50 of the patient, friction (e.g., traction) between the sole 410 and the ground may be higher than friction between the heel protector and the ground when the boot cover 400 is not installed. As a result, the boot cover 400 may prevent the patient from slipping during ambulation when the boot cover 400 is installed over the heel protector. In some embodiments, the sole 410 is formed out of a rubber material to provide the higher traction between the sole 410 and the ground. In some embodiments, the sole 410 includes traction features 412 (e.g., traction grooves, traction extrusions) configured to increase an amount of friction between the ground and the sole 410.

As shown in FIG. 10, the boot cover 400 includes a foot cover 414 configured to cover the foot portion 56 of the patient when the boot cover 400 is positioned on the lower limb 50 of the patient. The foot cover 414 and the sole 410 collectively define a foot opening configured to receive the foot portion 56 of the patient. The foot cover 414 may be configured to slip over the foot portion 56 of the patient when the foot portion 56 is inserted through an entrance (e.g., an aperture, an opening) to the foot opening to position the boot cover 400 on the lower limb 50 of the patient. For example, the foot portion 56 of the patient may be received by the foot opening of the boot cover 400 when the boot cover 400 is positioned on the foot portion 56 of the patient.

In some embodiments, the foot cover 414 includes an elastic portion 416 positioned around the entrance the foot opening. The elastic portion 416 may be configured to bias the entrance of the foot opening of the boot cover 400 closed. For example, the elastic portion 416 may be formed from an elastic material that is positioned around the entrance of the foot opening of the boot cover 400 and bias the entrance of the foot opening toward a closed configuration where the entrance of the foot opening does not allow access into the foot opening. The elastic portion 416 is configured to expand when the foot opening receives the foot portion 56. Once the foot opening has received the foot portion 56, the elastic portion 416 may contract around the lower limb 50 (e.g., around the ankle portion 60 of the lower limb 50) to prevent removal of the foot portion 56 from the foot opening to allow for the patient to ambulate without the boot cover 400 being removed from the lower limb 50. Additionally, the elastic portion 416 may be configured to expand to receive various sizes of heel protectors positioned on the lower limb 50 of the patient when the foot portion 56 is received by the foot cover 414. For example, the elastic portion 416 may need to expand further when the heel protector is a boot than when the heel protector is a heel wrap. As a result, the foot cover 414 may be configured to be positioned on the lower limb 50 when various configurations of the heel protectors are positioned on the lower limb 50.

### Overview of Universal Fit Positioning System

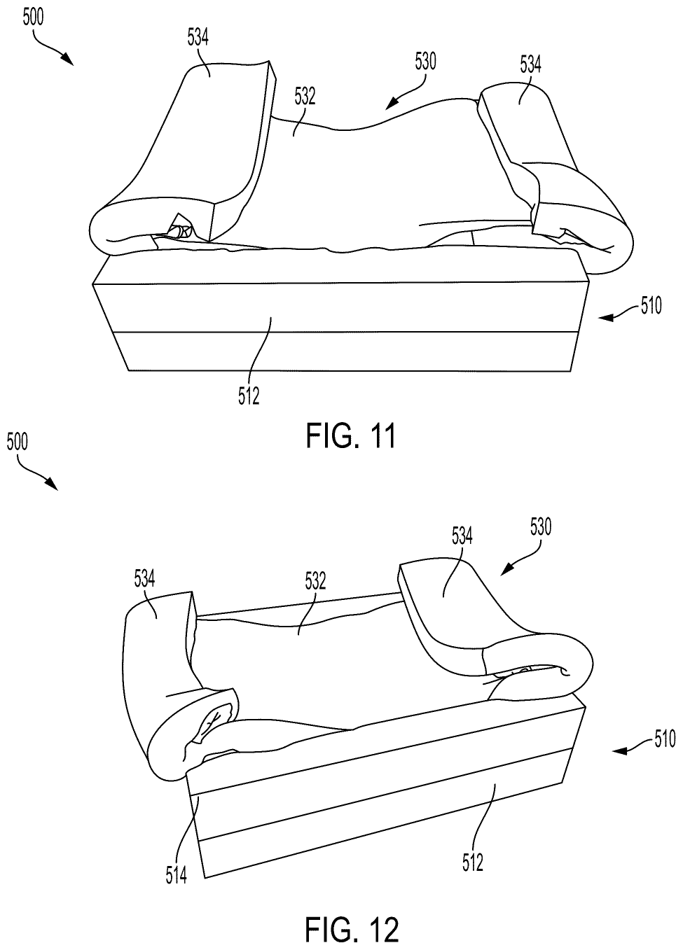

As shown in FIGS. 11-16, the lower limb positioning system 10 includes a universal fit positioning system 500 (e.g., a universal limb positioning system, a universal cushion system) configured to support limbs of patients, according to some embodiments. As shown in FIGS. 14-16, the universal fit positioning system 500 is configured to support both lower limbs 50 and upper limbs 70 (e.g., an arm) of patients. The universal fit positioning system 500 may be placed between a surface and the upper limb 70 or the lower limb 50 of the patient to provide a cushion and/or a support between the upper limb 70 or the lower limb 50 and the surface. For example, the universal fit positioning system 500 may be positioned between the upper limb 70 or the lower limb 50 and the surface to prevent pressure injuries from forming on the upper limb 70 or the lower limb 50. In some embodiments, the universal fit positioning system 500 is configured to support the upper limbs 70 and/or the lower limbs 50 of any patients that fall into the size range of a 1% percentile female to a 99% percentile male. For example, the universal fit positioning system 500 may be configured to fit sizes of the lower limb 50 ranging between a 1% percentile female's calf length of approximately 337 mm (e.g., 13.25″) and a 99% percentile male's calf length of approximately 495 mm (e.g., 19.48″). As another example, the universal fit positioning system 500 may be configured to fit sizes of the upper limb 70 ranging from a 1% female's elbow to wrist length of approximately 229 mm (e.g., 9.03″) to a 99% male's elbow to wrist length of approximately 328 mm (e.g., 12.93″).

As shown in FIGS. 11-16, the universal fit positioning system 500 includes a floater assembly 510 (e.g., a floater pad, a floater cushion) configured to be positioned on a surface below the patient and a pad assembly 530 (e.g., a pad cushion, a support cushion) configured to be positioned between the floater assembly 510 and the lower limb 50 or the upper limb 70 of the patient. The floater assembly 510 includes a base portion 512 configured to contact the surface and wall portions 514 extending upward from the base portion 512. The wall portions 514 may define an opening 516 configured to receive the pad assembly 530. For example, when the pad assembly 530 is received by the opening 516, the wall portions 514 may prevent the movement of the pad assembly 530 in a first direction while allowing the pad assembly 530 to move in a second direction perpendicular to the first direction. As a result, the pad assembly 530 may be adjusted in the second direction to allow for the different sizes of the lower limbs 50 and the upper limbs 70 to be accommodated by the universal fit positioning system 500, but may not be adjusted in the first direction to prevent the lower limb 50 or the upper limb 70 from falling off of the universal fit positioning system 500. In some embodiments, at least one surface of the floater assembly 510 (e.g., a bottom surface) is formed and/or coated with a material with a high coefficient of friction (e.g., higher than the surface under the patient). For example, the surface of the floater assembly 510 may be formed and/or coated with silicone rubber, neoprene, polyurethane foam, or other materials with coefficients of friction that are higher than standard bed sheets on a hospital bed. When the patient is positioned on the surface and the universal fit positioning system 500 is positioned between the lower limb 50 or the upper limb 70 of the patient and the surface, the high coefficient of friction of the floater assembly 510 may reduce the possibility of the floater assembly 510 moving relative to the surface.

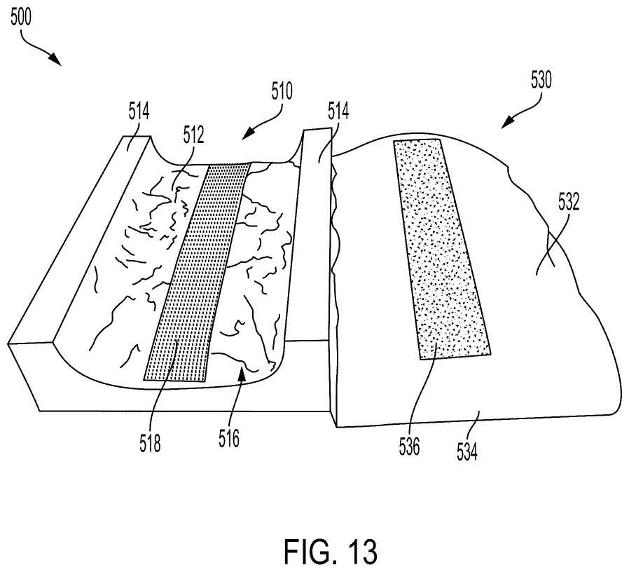

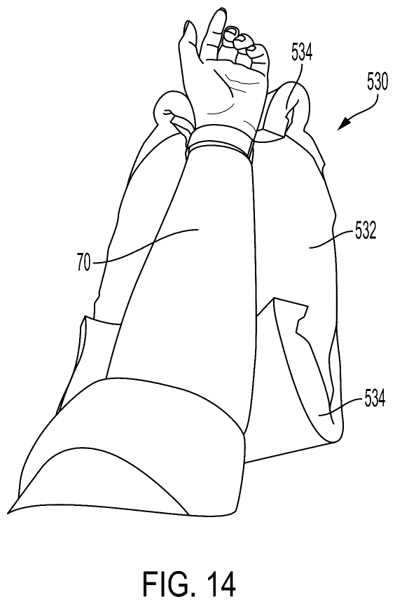

As shown in FIG. 13, the floater assembly 510 includes a mounting portion 518 configured to releasably couple to the pad assembly 530. For example, the mounting portion 518 may be a first portion of a hook-and-loop fastener (e.g., a hook portion, a loop portion) configured to engage a second portion of the hook-and-loop fastener of the pad assembly 530 to releasably couple the pad assembly 530 to the floater assembly 510.

The pad assembly 530 includes a pad base 532 configured to support the lower limb 50 or the upper limb 70 of the patient. The pad assembly 530 may be positioned between the floater assembly 510 and the lower limb 50 or the upper limb 70 of the patient or the pad assembly 530 may be positioned on the surface below the patient between the surface and the lower limb 50 or the upper limb 70 (e.g., when the floater assembly 510 is not used). For example, the pad assembly 530 may be released from the floater assembly 510 (e.g., removed from the mounting portion 518) and positioned under the heel portion 58 of the lower limb 50 of the patient or an elbow of the upper limb 70 of the patient separate from the floater assembly 510 to cushion the heel portion 58 or the elbow. The pad assembly 530 may be wrapped around the heel portion 58 or the elbow of the patient when the pad assembly 530 is separate from the floater assembly 510 to act as a heel wrap or an elbow wrap.

The pad assembly 530 includes a pair of flex ends 534 (e.g., a plurality of the flex ends 534) positioned at opposing ends of the pad base 532. For example, a first of the flex ends 534 may be positioned at a first end of the pad base 532 and a second of the flex ends 534 may be positioned at an opposing second end of the pad base 532. When the pad assembly 530 is received by the opening 516 defined by the floater assembly 510, the pair of the flex ends 534 may be positioned on the opposing ends of the pad base 532 in the second direction that the floater assembly 510 allows the pad assembly 530 to be adjusted. In some embodiments, the flex ends 534 curl outward and upward from the pad base 532 back toward a center of the pad base 532 (e.g., toward the other of the flex ends 534) to provide additional cushioning to the lower limb 50 or the upper limb 70. For example, by folding back toward the center of the pad base 532, the flex ends 534 may be thicker than the center of the pad base 532 to provide the additional cushioning to the lower limb 50 or the upper limb 70. In other embodiments, the flex ends 534 are formed to be thicker than the pad base 532 to provide the additional cushioning to the lower limb 50 or the upper limb 70. In some embodiments, the flex ends 534 and the pad base 532 may be integrally formed (e.g., formed out of a single piece of material). In other embodiments, the flex ends 534 are coupled to the opposing ends of the pad base 532.

As shown in FIG. 13, the pad assembly 530 includes a coupling portion 536 configured to be releasably coupled to the mounting portion 518 to releasably couple the pad assembly 530 to the floater assembly 510. The coupling portion 536 may be configured to releasably couple the pad assembly 530 to the floater assembly 510 in a plurality of different configurations to allow for the pad assembly 530 to be positioned in a plurality of different positions relative to the floater assembly 510 (e.g., in the second direction that the floater assembly 510 allows the pad assembly 530 to be adjusted). For example, the coupling portion 536 may be the second portion of the hook-and-loop fastener (e.g., the hook portion when the mounting portion 518 is configured as the loop portion, the loop portion when the mounting portion 518 is configured as the hook portion) configured to engage the mounting portion 518 configured as the first portion of the hook-and-loop fasteners to releasably couple the pad assembly 530 to the floater assembly 510. The hook-and-loop fastener may allow for the coupling portion 536 to be releasably coupled to the mounting portion 518 in a plurality of different positions along a length of the mounting portion 518 to allow for the coupling portion 536 to releasably couple the pad assembly 530 to the floater assembly 510 in the different configurations. By allowing for the different configurations of the pad assembly 530 relative to the floater assembly 510, the universal fit positioning system 500 may accommodate the wide range of sizes of the lower limb 50 and the upper limb 70 based on different sizes of the patients.

In some embodiments, the pad assembly 530 is formed and/or coated with materials that are easy to clean (e.g., materials that are compatible with cleaning products, materials that can be run through a laundry machine). For example, the pad assembly 530 may be formed and/or coated with silicone, nylon, polyester, or other materials that are easy to sterilize (e.g., chemically sterilize, sterilize using high temperatures), chemically resistant, and/or able to corrosion resistant. In some embodiments, the pad assembly 530 is formed and/or coated with materials that have antiseptic properties (e.g., materials that kill pathogens on contact, materials that are not conducing to hosting pathogens). For example, the pad assembly 530 may be formed and/or coated with silver nanoparticles, graphene oxide, or other materials that are detrimental to pathogens. In some embodiments, the pad assembly 530 is formed and/or coated with materials with a high durability (e.g., materials that can retain desired properties through multiple uses). For example, the pad assembly 530 may be formed and/or coated with polyester, Kevlar, nylon, or other materials that can withstand repeated uses.

In some embodiments, a portion of the pad assembly 530 (e.g., the pad base 532, the flex ends 534) is formed out of a cushioning material configured to distribute pressure around the lower limb 50 or the upper limb 70 of the patient when the lower limb 50 or the upper limb 70 are supported by the pad assembly 530. For example, the portion of the pad assembly 530 may be formed out of polyurethane foam, polyethylene foam, silicone foam, or other materials that provide cushioning and support. By cushioning the lower limb 50 or the upper limb 70, the pad assembly 530 may prevent injuries to the lower limb 50 or the upper limb 70 such as pressure injuries. In some embodiments, the portion of the pad assembly 530 is formed out of a memory foam (e.g., a viscoelastic foam). The memory foam may be a highly supportive foam that is configured to conform to a profile (e.g., a shape) of the lower limb 50 or the upper limb 70 when supporting the lower limb 50 or the upper limb 70 to evenly distribute weight, alleviate pressure points, and improve blood circulation of the lower limb 50 or the upper limb 70. The memory foam may also be a breathable foam configured dissipate heat received from the lower limb 50 or the upper limb 70, which may prevent heat injuries to the lower limb 50 or the upper limb 70.

### Overview of Modular Protector

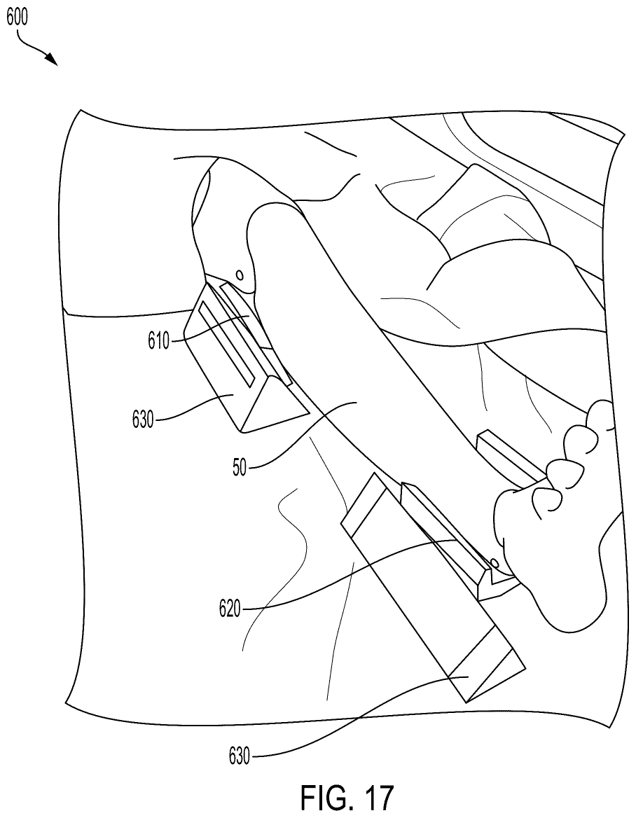

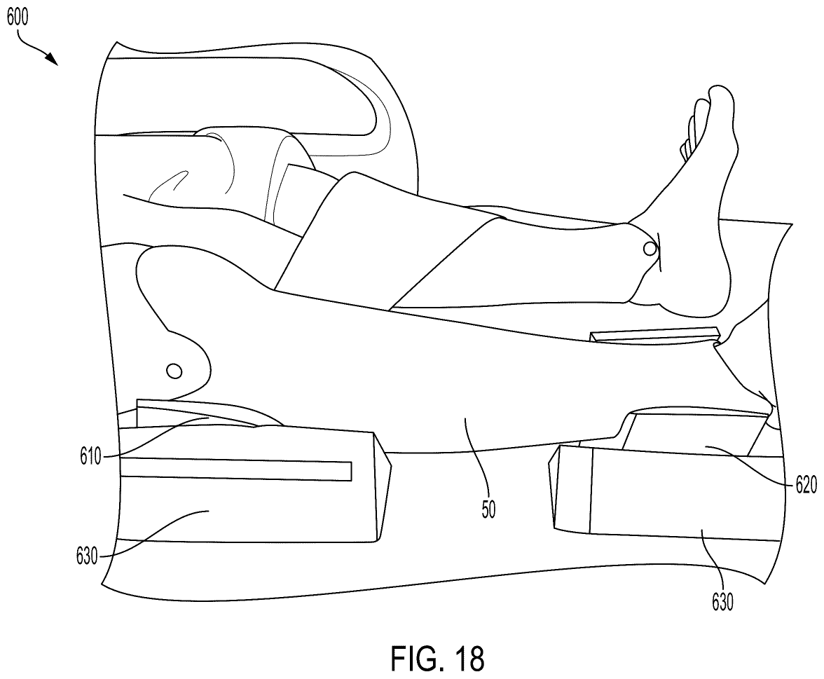

As shown in FIGS. 17 and 18, the lower limb positioning system 10 includes a modular positioning system 600 (e.g., a modular limb positioning system, a modular cushion system) configured to support the lower limbs 50 of patients, according to some embodiments. The modular positioning system 600 includes a plurality of modules (e.g., cushioning modules, support modules) configured to be individually (e.g., separately) positioned between a surface and the lower limb 50 of the patient to provide cushioning and/or support for the lower limb 50 of the patient. For example, the modules of the modular positioning system 600 may be individually positioned between the lower limb 50 and the surface to prevent pressure injuries from forming on the lower limb 50.

By allowing for the modules of the modular positioning system 600 to be individually positioned between the surface and the lower limb 50 of the patient, the modular positioning system 600 may allow for customization for the patient supported by the modular positioning system 600. For example, if the patient has a sore at a location of the lower limb 50 of the patient, the modules of the modular positioning system 600 may be positioned between the lower limb 50 and the surface such that the modules do not contact the location of the lower limb 50 with the sore to reduce the discomfort of the patient. Additionally, the individual positioning of the modules of the modular positioning system 600 may allow for gaps between the modules of the modular positioning system 600 while the modules are supporting the lower limb 50 of the patient, which may allow for easy access to the lower limb 50 for monitoring and inspection and may allow for air flow around the lower limb 50 of the patient to allow for temperature regulation of the lower limb 50.

As shown in FIGS. 17 and 18, the modular positioning system 600 includes a knee module 610 (e.g., a knee cushion, a knee pad) configured to be positioned on a surface under a knee of the lower limb 50 and a foot module 620 (e.g., a foot cushion, a foot pad) configured to be positioned on the surface under the foot portion 56 of the lower limb 50. The knee module 610 may be positioned between the surface and the knee of the lower limb 50 to provide cushioning and/or support for the knee of the lower limb 50. The foot module 620 may be positioned between the surface and the foot portion 56 of the lower limb 50 of the patient to provide cushioning and/or support for the foot portion 56 of the lower limb 50 of the patient. In various embodiments, the modular positioning system 600 includes additional modules configured to be positioned on the surface under other portions of the lower limb 50 of the patient. For example, the modular positioning system 600 may include a calf module configured to be positioned between the surface and a calf of the lower limb 50 and/or a thigh module configured to be positioned between the surface and a thigh of the lower limb 50.

In some embodiments, the modules of the modular positioning system 600 are formed and/or coated with materials that are easy to clean (e.g., materials that are compatible with cleaning products, materials that can be run through a laundry machine). For example, the modules of the modular positioning system 600 may be formed and/or coated with silicone, nylon, polyester, or other materials that are easy to sterilize (e.g., chemically sterilize, sterilize using high temperatures), chemically resistant, and/or able to corrosion resistant. In some embodiments, the modules of the modular positioning system 600 are formed and/or coated with materials that have antiseptic properties (e.g., materials that kill pathogens on contact, materials that are not conducing to hosting pathogens). For example, the modules of the modular positioning system 600 may be formed and/or coated with silver nanoparticles, graphene oxide, or other materials that are detrimental to pathogens. In some embodiments, the modules of the modular positioning system 600 are formed and/or coated with materials with a high durability (e.g., materials that can retain desired properties through multiple uses). For example, the modules of the modular positioning system 600 may be formed and/or coated with polyester, Kevlar, nylon, or other materials that can withstand repeated uses.

In some embodiments, the modules of the modular positioning system 600 include a cushioning material configured to distribute pressure around the lower limb 50 of the patient. For example, the modules of the modular positioning system 600 may include polyurethane foam, polyethylene foam, silicone foam, or other materials that provide cushioning and support. As another example, the modules of the modular positioning system 600 may include a foam material such as memory foam. In some embodiments, at least a portion of the modules of the modular positioning system 600 are formed and/or coated with a material with a high coefficient of friction (e.g., higher than the surface under the patient). For example, the modules of the modular positioning system 600 may be formed and/or coated with silicone rubber, neoprene, polyurethane foam, or other materials with coefficients of friction that are higher than standard bed sheets on a hospital bed. When the patient is positioned on the surface and the knee module 610 is positioned between the surface and the knee of the lower limb 50 of the patient, the high coefficient of friction of the portion of the knee module 610 may reduce the possibility of the knee module 610 moving relative to the surface.

In some embodiments, modules of the modular positioning system 600 are also configured to be positioned on the surface under portions of the upper limb 70 of the patient. For example, the knee module 610 may also be configured to be positioned on the surface under an elbow of the upper limb 70 of the patient and the foot module 620 may also be configured to be positioned on the surface under a hand of the upper limb 70 of the patient to provide cushioning and/or support to the upper limb 70 of the patient.

In some embodiments, the modules of the modular positioning system 600 are held in position by wedge modules 630 (e.g., wedges, positioning wedges). The wedge modules 630 are configured to be inserted at least partially under the modules of the modular positioning system 600 and/or other components of the lower limb positioning system 10 so provide additionally cushioning and/or support to the lower limb 50 and/or the upper limb 70 of the patient. The wedge modules 630 may have a wedge shape (e.g., a triangular cross section) such that one of the tips of the wedge modules 630 may be inserted under the modules of the modular positioning system 600 to provide support under the modules of the modular positioning system 600. For example, the tips of the wedge modules 630 may be inserted between the modules of the modular positioning system 600 and the surface under the patient to provide additional cushioning and/or support between the modules of the modular positioning system 600 and the surface. The wedge modules 630 may include a cushioning material configured to distribute pressure received by the wedge modules 630. For example, the wedge modules 630 may include polyurethane foam, polyethylene foam, silicone foam, or other materials that provide cushioning and support.

In some embodiments, the wedge modules 630 are configured to restrict movement of the lower limb 50 and/or the upper limb 70 relative to the surface below the patient. For example, when the wedge modules 630 are inserted under the modules of the modular positioning system 600, the wedge modules 630 may restrict the movement of the modules of the modular positioning system 600 relative to the surface such that the movement of the lower limb 50 and/or the upper limb 70 is restricted relative to the surface. In some embodiments, at least one surface of the wedge modules 630 is formed and/or coated with a material with a high coefficient of friction (e.g., higher than the surface under the patient, higher than surfaces of the modules of the modular positioning system 600). For example, the surface of the wedge modules 630 may be formed and/or coated with silicone rubber, neoprene, polyurethane foam, or other materials with coefficients of friction that are higher than standard bed sheets on a hospital bed.

### Overview of Wedge Cushioning System

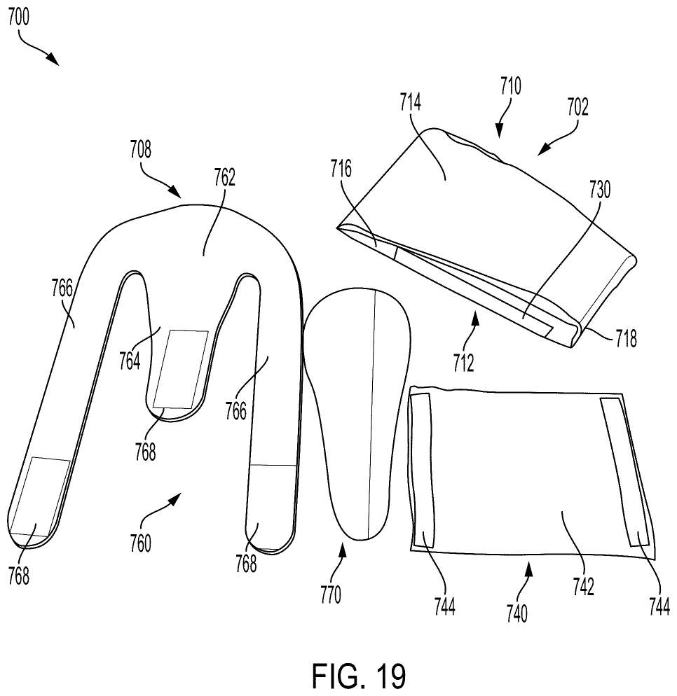

As shown in FIG. 19, the lower limb positioning system 10 includes a wedge cushioning system 700 (e.g., heel cushioning system a heel protection system, a heel positioning system) configured to cushion and/or support the heel portion 58 of the lower limbs 50 of patients, according to some embodiments. For example, when a patient is positioned on a bed the lower limb 50 of the patient may be positioned on the wedge cushioning system 700 to offload the heel portion 58 of the lower limb 50 of the patient from a surface of the bed. In some embodiments, the wedge cushioning system 700 utilizes a combination of reusable and disposable components to cushion and/or support the heel portion 58 of the lower limbs 50 of patients.

In some embodiments, the materials used in the wedge cushioning system 700 are breathable and are intended to keep the microclimate of the lower limb 50 cool and dry, since heat and moisture are contributing factors in pressure injuries. For example, the wedge cushioning system 700 may include materials such as polyester, nylon, polypropylene, or other materials that allow for air and/or moisture to transfer through the materials to dry and/or cool the lower limb 50 of the patient, When the wedge cushioning system 700 is used to cushion and/or support the heel portion 58, the heel portion 58 may be visible for skin assessments conducted by nurses. Additionally, the wedge cushioning system 700 may cover less of the lower limb 50 of the patient when cushioning and/or supporting the lower limb 50, which may allow for additional temperature regulation of the lower limb 50 to keep the lower limb 50 of the patient from overheating.

As shown in FIG. 19, the wedge cushioning system 700 includes a wedge assembly 702 (e.g., a wedge, a wedge support) configured to be positioned on a surface under the heel portion 58 of the lower limb 50 of the patient and a heel wrap assembly 708 (e.g., a heel wrap) configured to be wrapped around the heel portion 58 of the patient. The wedge assembly 702 includes a wedge tip end 704 (e.g., a distal end) configured to be positioned under the heel portion 58 of the lower limb 50 of the patient and a wedge base end 706 (e.g., a proximal end) configured to be positioned under a portion of the lower limb 50 proximal to the heel portion 58 of the lower limb 50. When the heel wrap assembly 708 is wrapped around the heel portion 58 and the heel portion 58 is supported by the wedge assembly 702, a portion of the heel wrap assembly 708 may be positioned between the heel portion 58 and the wedge assembly 702.

As shown in FIG. 19, the wedge assembly 702 includes a wedge pad 710 (e.g., a solid wedge, wedge) configured to cushion and/or support the heel portion 58 of the lower limb 50 of the patient and a strap 740 (e.g., a retention strap, a spandex strap) configured to releasably couple to the wedge pad 710 to couple the lower limb 50 of the patient to the wedge pad 710, according to some embodiments. In some embodiments, the wedge assembly 702 includes a plurality of the straps 740 configured to couple the lower limb 50 of the patient to the wedge pad 710. The wedge pad 710 may decrease in thickness from the wedge base end 706 of the wedge assembly 702 to the wedge tip end 704 of the wedge assembly 702. For example, a first thickness of the wedge pad 710 at a first location along a length of the wedge pad 710 may be greater than a second thickness of the wedge pad 710 at a second location along the length of the wedge of the wedge pad 710 positioned closer to the wedge tip end 704 than the first location. As another example, the wedge pad 710 may slope downwardly from the wedge base end 706 of the wedge assembly 702 towards the wedge tip end 704 of the wedge assembly 702. The profile of the wedge pad 710 may be configured to evenly distribute the weight of the lower limb 50 of along the length of the wedge pad 710 such that a pressure hot spot does not form on the lower limb 50. In some embodiments, the corners of the wedge pad 710 at the wedge tip end 704 and/or the proximal end are rounded. The rounded corners of the wedge pad 710 may decrease a foot print of the wedge pad 710 to allow for the wedge pad 710 to take up less space.

The wedge pad 710 may be configured as a reusable component (e.g., a component that can be used for multiple patients) of the wedge cushioning system 700. In some embodiments, when the wedge pad 710 is configured as a reusable component, the wedge pad 710 may be formed and/or coated with materials that are easy to clean (e.g., materials that are compatible with cleaning products, materials that can be run through a laundry machine) so that the wedge pad 710 can be cleaned between uses. For example, the wedge pad 710 may be formed and/or coated with silicone, nylon, polyester, or other materials that are easy to sterilize (e.g., chemically sterilize, sterilize using high temperatures), chemically resistant, and/or able to corrosion resistant. Since the wedge pad 710 may have incidental direct contact with the lower limb 50 of the patient, cleaning and reusing the wedge pad 710 may be a simple procedure conducted by a care facility (e.g., a hospital, a retirement home) caring for the patient. For example, the procedure for cleaning and reusing the wedge pad 710 may be as simple as wiping down the wedge pad 710 with an antiseptic wipe.

In some embodiments, when the wedge pad 710 is configured as a reusable component, the wedge pad 710 may be formed and/or coated with materials that have antiseptic properties (e.g., materials that kill pathogens on contact, materials that are not conducing to hosting pathogens) such that pathogens are not transferred between patients when the wedge pad 710 is reused for different patients. For example, the cushioning boot 100 may be formed and/or coated with silver nanoparticles, graphene oxide, or other materials that are detrimental to pathogens. In some embodiments, when the wedge pad 710 is configured as reusable component, the wedge pad 710 may be formed and/or coated with materials with a high durability (e.g., materials that can retain desired properties through multiple uses) such that the wedge pad 710 does not degrade over multiple uses. For example, the wedge pad 710 may be formed and/or coated with polyester, Kevlar, nylon, or other materials that can withstand repeated uses. In other embodiments, the wedge pad 710 may be configured as a single use component configured to be used to support the heel portion 58 of the patient for a single time period or support the heel portion 58 of only one of the patients. When the wedge pad 710 is configured as a single use component, the wedge pad 710 may be formed and/or coated with materials that are difficult to clean or fragile. For example, the wedge pad 710 may be coated with fleece to increase a comfort of the patient. However, since fleece may be difficult to clean, the wedge pad 710 may be replaced between uses. As another example, the wedge pad 710 may be formed out of cotton sheeting material that may easily rip or tear.

As shown in FIGS. 19-22, the wedge pad 710 includes a wedge bottom panel 712 (e.g., a wedge lower panel) extending from the wedge tip end 704 of the wedge assembly 702 to the wedge base end 706 of the wedge assembly 702 and configured to contact the surface below the patient. The wedge bottom panel 712 may define a wedge bottom surface extending along the wedge bottom panel 712. In some embodiments, the wedge bottom panel 712 is formed and/or coated with a material with a low coefficient of friction (e.g., lower than the surface under the patient). For example, the wedge bottom panel 712 may be formed and/or coated with nylon, silicone, nitrile, or other materials with coefficients of friction that are lower than standard bed sheets on a hospital bed. When the wedge bottom panel 712 is positioned on the surface and the lower limb 50 of the patient is supported by the wedge pad 710, the low coefficient of friction of the wedge bottom panel 712 may allow for adjustment of the position of the wedge pad 710 relative to the surface without having to lift the lower limb 50 off of the wedge pad 710.

As shown in FIGS. 19-21 and 23, the wedge pad 710 includes a wedge top panel 714 (e.g., a wedge upper panel) extending from the wedge tip end 704 of the wedge assembly 702 to the wedge base end 706 of the wedge assembly 702 and configured to contact the heel wrap assembly 708 when the heel wrap assembly 708 is positioned on the lower limb 50 of the patient and the lower limb 50 is supported by the wedge pad 710. The wedge top panel 714 may define a wedge top surface extending along the wedge top panel 714. In some embodiments, the wedge top panel 714 is formed and/or coated with a material with a high coefficient of friction (e.g., higher than the surface under the patient, higher than the coefficient of friction of the wedge bottom panel 712). For example, the wedge top panel 714 may be formed and/or coated with silicone rubber, neoprene, polyurethane foam, or other materials with coefficients of friction that are higher than standard bed sheets on a hospital bed. When the wedge top panel 714 supports the lower limb 50 of the patient (e.g., through the heel wrap assembly 708), the high coefficient of friction of the wedge top panel 714 may prevent the lower limb 50 from sliding across the wedge top panel 714.

As shown in FIGS. 19-22, the wedge pad 710 includes a pair of wedge side panels 716 coupled between the wedge bottom panel 712 and the wedge top panel 714. For example, a first of the wedge side panel 716 may be coupled between the wedge bottom panel 712 and the wedge top panel 714 on a left side of the wedge pad 710 and a second of the wedge side panel 716 may be coupled between the wedge bottom panel 712 and the wedge top panel 714 on the right side of the wedge pad 710. The wedge side panels 716 may define wedge side surfaces extending along each of the wedge side panels 716. The wedge side surface may be oriented perpendicular to the wedge top surface and/or the wedge bottom surface.

In some embodiments, the wedge side panel 716 extend from the wedge tip end 704 of the wedge assembly 702 to the wedge base end 706 of the wedge assembly 702. A height of the wedge side panel 716 may increase as the wedge side panel 716 extend from the wedge tip end 704 of the wedge assembly 702 to the wedge base end 706 of the wedge assembly 702. For example, a first height of the wedge side panel 716 at a first location along the length of the wedge assembly 702 may be greater than a second height of the wedge side panel 716 at a second location along the length of the wedge assembly 702 positioned closer to the wedge tip end 704 of the wedge assembly 702 than the first location. In other embodiments, the wedge side panel 716 couple the wedge bottom panel 712 to the wedge top panel 714 proximate the wedge base end 706 of the wedge assembly 702 and the wedge bottom panel 712 is directly coupled to the wedge top panel 714 proximate the wedge tip end 704 of the wedge assembly 702. In still other embodiments, the wedge pad 710 does not include the wedge side panel 716 and the wedge bottom panel 712 is directly coupled to the wedge top panel 714 along the length of the wedge pad 710 on the left side and the right side of the wedge pad 710.

In some embodiments, the wedge side panel 716 are formed out of or coated with the same material as the wedge bottom panel 712. For example, the wedge side panel 716 may be formed from or coated with the same material with the low coefficient of friction as the wedge bottom panel 712. When the wedge side panel 716 are formed out of the same material as the wedge bottom panel 712, the wedge side panel 716 and the wedge bottom panel 712 may be integrally formed (e.g., formed out of a single piece of the material, formed out of the same piece of fabric) or the wedge side panel 716 and the wedge bottom panel 712 may be formed out of different pieces of the material and coupled together (e.g., sewn together, soldered together). In other embodiments, the wedge side panel 716 are formed out of or coated with the same material as the wedge top panel 714. For example, the wedge side panel 716 may be formed from or coated with the same material with the high coefficient of friction as the wedge top panel 714. When the wedge side panel 716 are formed out of the same material as the wedge top panel 714, the wedge side panel 716 and the wedge top panel 714 may be integrally formed or the wedge side panel 716 and the wedge bottom panel 712 may be formed out of different pieces of the material and coupled together.

As shown in FIGS. 19-21 and 23, the wedge pad 710 includes a wedge base panel 718 coupled between the wedge bottom panel 712 and the wedge top panel 714 at the wedge base end 706 of the wedge assembly 702. The wedge base panel 718 may define a wedge rear surface extending along the wedge base panel 718. The wedge rear surface may be oriented perpendicular to the wedge top surface, the wedge bottom surface, and/or the wedge side surface. In some embodiments, the wedge base panel 718 is coupled between a first of the wedge side panels 716 and a second of the wedge side panels 716 at the wedge base end 706 of the wedge assembly 702. For example, the wedge base panel 718 may extend across a width of the wedge pad 710 between the first of the wedge side panels 716 and the second of the wedge side panels 716. A height of the wedge base panel 718 may be equal to the height of the wedge pad 710 at the wedge base end 706 of the wedge assembly 702.

In some embodiments, the wedge base panel 718 is formed out of or coated with the same material as the wedge bottom panel 712. For example, the wedge base panel 718 may be formed from or coated with the same material with the low coefficient of friction as the wedge bottom panel 712. When the wedge base panel 718 is formed out of the same material as the wedge bottom panel 712, the wedge base panel 718 and the wedge bottom panel 712 may be integrally formed or the wedge base panel 718 and the wedge bottom panel 712 may be formed out of different pieces of the material and coupled together. In other embodiments, the wedge base panel 718 is formed out of or coated with the same material as the wedge top panel 714. For example, the wedge base panel 718 may be formed from or coated with the same material with the high coefficient of friction as the wedge top panel 714. When the wedge base panel 718 is formed out of the same material as the wedge top panel 714, the wedge base panel 718 and the wedge top panel 714 may be integrally formed or the wedge base panel 718 and the wedge top panel 714 may be formed out of different pieces of the material and coupled together.

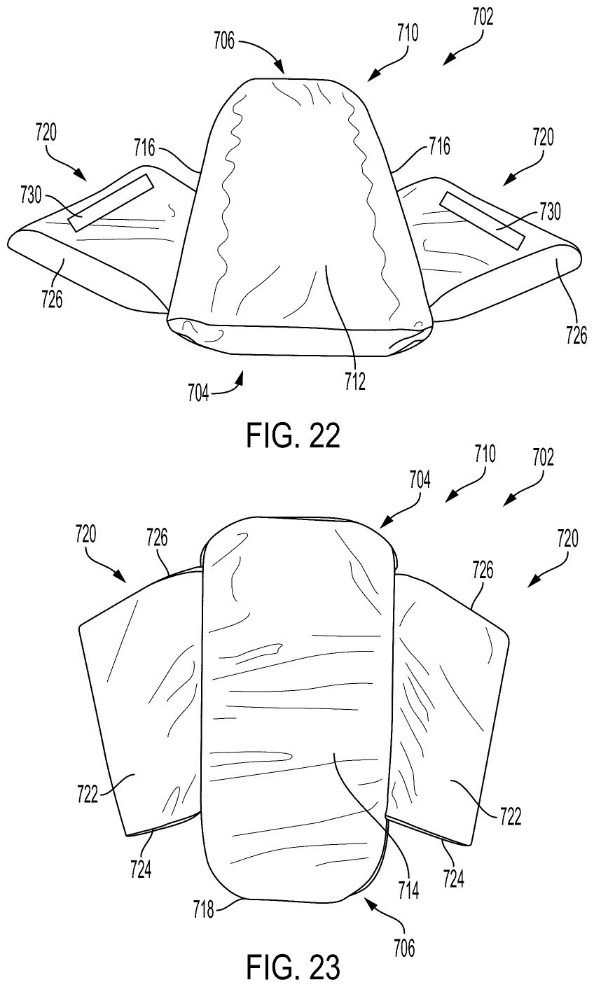

As shown in FIGS. 22-25, the wedge assembly 702 includes a plurality of wedge wings 720 (e.g., wings, containment wings, enclosures, a first wedge wing and a second wedge wing), each coupled to one of the wedge side panels 716, according to some embodiments. The wedge wings 720 may be configured to be moved between a lowered position and a raised position where the wedge wings 720 extend higher than when the wedge wings 720 are in the lowered position. In some embodiments, the wedge wings 720 may be pivotably coupled to the wedge side panels 716 and may be pivoted between the lowered position and the raised position. For example, a lowered orientation of the wedge side panels 716 in the lowered position may be angularly offset (e.g., by 45 degrees, by 75 degrees, by 90 degrees) from a raised orientation of the wedge side panels 716 in the raised position.

The wedge wings 720 may be configured as reusable components of the wedge cushioning system 700. In some embodiments, when the wedge wings 720 are configured as a reusable component, the wedge wings 720 may be formed and/or coated with materials that are easy to clean (e.g., materials that are compatible with cleaning products, materials that can be run through a laundry machine) so that the wedge wings 720 can be cleaned between uses. For example, the wedge wings 720 may be formed and/or coated with silicone, nylon, polyester, or other materials that are easy to sterilize (e.g., chemically sterilize, sterilize using high temperatures), chemically resistant, and/or able to corrosion resistant. Since the wedge wings 720 may have incidental direct contact with the lower limb 50 of the patient, cleaning and reusing the wedge wings 720 may be a simple procedure conducted by a care facility (e.g., a hospital, a retirement home) caring for the patient. For example, the procedure for cleaning and reusing the wedge wings 720 may be as simple as wiping down the wedge wings 720 with an antiseptic wipe.

In some embodiments, when the wedge wings 720 are configured as a reusable component, the wedge wings 720 may be formed and/or coated with materials that have antiseptic properties (e.g., materials that kill pathogens on contact, materials that are not conducing to hosting pathogens) such that pathogens are not transferred between patients when the wedge wings 720 is reused for different patients. For example, the wedge wings 720 may be formed and/or coated with silver nanoparticles, graphene oxide, or other materials that are detrimental to pathogens. In some embodiments, when the wedge wings 720 are configured as reusable component, the wedge wings 720 may be formed and/or coated with materials with a high durability (e.g., materials that can retain desired properties through multiple uses) such that the wedge wings 720 do not degrade over multiple uses. For example, the wedge wings 720 may be formed and/or coated with polyester, Kevlar, nylon, or other materials that can withstand repeated uses. In other embodiments, the wedge wings 720 may be configured as a single use components configured to be used to receive the lower limb 50 of the patient for a single time period or support the heel portion 58 of only one of the patients. When the wedge wings 720 are configured as single use components, the wedge wings 720 may be formed and/or coated with materials that are difficult to clean or fragile. For example, the wedge wings 720 may be coated with fleece to increase a comfort of the patient. However, since fleece may be difficult to clean, the wedge wings 720 may be replaced between uses. As another example, the wedge wings 720 may be formed out of cotton sheeting material that may easily rip or tear.

In some embodiments, the wedge wings 720 are formed and/or coated with a material with a high coefficient of friction (e.g., higher than the surface under the patient, higher than the coefficient of friction of the wedge bottom panel 712,). For example, the wedge wings 720 may be formed and/or coated with silicone rubber, neoprene, polyurethane foam, or other materials with coefficients of friction that are higher than standard bed sheets on a hospital bed. When the lower limb 50 of the patient is supported by the wedge pad 710 and the wedge wings 720 are in the raised position, the high coefficient of friction of the wedge wings 720 may allow for the wedge wings 720 to restrict movement of the lower limb 50 relative to the wedge wings 720. In various embodiments, the wedge wings 720 are formed out of or coated with the same material as the wedge bottom panel 712 and/or the wedge top panel 714. For example, the wedge wings 720 may be formed from or coated with the same material with the low coefficient of friction as the wedge bottom panel 712 or the same material with the high coefficient of friction as the wedge top panel 714.

When the wedge wings 720 are in the lowered position, the wedge assembly 702 may be placed in a base configuration. In the base configuration, the wedge assembly 702 may be configured to receive the lower limb 50 of the patient. For example, when the wedge wings 720 are in the lowered position, the lower limb 50 of the patient may be slid onto the wedge top surface of the wedge top panel 714 without having to lift the lower limb 50 of the patient over the wedge wings 720 in the raised position, which would result in having to lift the lower limb 50 higher. Once the lower limb 50 is supported by the wedge top panel 714, the wedge wings 720 may be moved into the raised position to place the wedge assembly 702 in a raised configuration. In the raised configuration, the wedge wings 720 are positioned on opposing sides of the lower limb 50 (e.g., a right side and a left side of the lower limb 50) and may restrict movement of the lower limb 50. For example, if the wedge wings 720 are positioned on the right side and the left side of the lower limb 50 in the raised position, the wedge wings 720 may restrict the movement of the lower limb 50 towards the right and towards the left to ensure that the lower limb 50 is not moved into a non-ideal configuration which could cause injury to the lower limb 50.

As shown in FIG. 23, the wedge wings 720 defines an inside wing inside surface 722 configured to restrict the movement of the lower limb 50 when the lower limb 50 is supported by the wedge pad 710 and the wedge wings 720 are in the raised position. For example, when the lower limb 50 attempts moves in one of the directions restricted by the wedge wings 720, the lower limb 50 may contact the inside wing inside surface 722 of one of the wedge wings 720. In some embodiments, when the wedge wings 720 are in the lowered position, the inside wing inside surface 722 is co-planer with the wedge top surface defined by the wedge top panel 714. As a result, when the lower limb 50 is being moved onto the wedge pad 710, the lower limb 50 may be slide along one of the inside wing inside surface 722 onto the wedge top panel 714 without an elevation (e.g., a height) of the lower limb 50 being changed, which may ease the process of positioning the lower limb 50 on the wedge pad 710 and/or increase a comfort of the patient while the lower limb 50 is being positioned on the wedge pad 710.

As shown in FIGS. 22 and 23, the wedge wings 720 define a wing tip edge 724 (e.g., a wing distal end) along a first end of the wedge wings 720 closest to the wedge tip end 704 of the wedge assembly 702. In some embodiments, the wing tip edge 724 angles toward the wedge base end 706 of the wedge assembly 702 as the wing tip edge 724 extends outward from the wedge side panel 716. In other embodiments, the wing tip edge 724 angles toward the wedge tip end 704 of the wedge assembly 702 as the wing tip edge 724 extends outward from the wedge side panel 716. In still other embodiments, the wing tip edge 724 does not angle toward the wedge tip end 704 or the wedge base end 706 of the wedge assembly 702 as the wing tip edge 724 extends outward from the wedge side panel 716. For example, the wing tip edge 724 may be oriented perpendicular to the wedge side surface defined by the wedge side panel 716 when the wedge wing 720 is in the lowered position.

As shown in FIGS. 22 and 23, the wedge wings 720 defines a wing base edge 726 (e.g., a wing proximal end edge) along a second end of the wedge wings 720 closest to the wedge base end 706 of the wedge assembly 702. In some embodiments, the wing base edge 726 angles toward the wedge base end 706 of the wedge assembly 702 as the wing tip edge 724 extends outward from the wedge side panel 716. In other embodiments, the wing base edge 726 angles toward the wedge tip end 704 of the wedge assembly 702 as the wing base edge 726 extends outward from the wedge side panel 716. In still other embodiments, the wing base edge 726 does not angle toward the wedge tip end 704 or the wedge base end 706 of the wedge assembly 702 as the wing base edge 726 extends outward from the wedge side panel 716.

As shown in FIGS. 19-22, the wedge assembly 702 includes a pair of mounting portions 730 configured to releasably couple to the strap 740. For example, the mounting portions 730 may each be a first portion of a hook-and-loop fastener (e.g., a hook portion, a loop portion) configured to engage second portions of the hook-and-loop fastener of the strap 740 to releasably couple the strap 740 to the wedge pad 710 and couple the lower limb 50 of the patient to the wedge pad 710 when the wedge pad 710 is supporting the lower limb 50 of the patient. As other examples the mounting portions 730 may each be a first portion of a button fastener (e.g., a male button, a female button), a first portion of an adhesive fastener (e.g., an adhesive portion, an adhesive receiving portion), a first portion of a clip fastener (e.g., a male clip, a female clip), etc.

As shown in FIGS. 19-21, each of the mounting portions 730 are positioned on one of the wedge side panel 716, according to some embodiments. For example, one of the mounting portions 730 may be coupled to each of the wedge side panel 716 such that the strap 740 may be releasably coupled to the wedge pad 710 proximate each of the wedge side panel 716. As shown in FIG. 22, each of the mounting portions 730 are positioned on one of the wedge wings 720, according to some embodiments. For example, one of the mounting portions 730 may be coupled to an outside surface of each of the wedge wings 720 opposing the inside wing inside surface 722 such that the strap 740 may be releasably coupled to each of the wedge wings 720. In some embodiments, when the strap 740 is coupled to each of the mounting portions 730 positioned on the wedge wings 720 when the wedge wings 720 are in the raised position, the strap 740 holds the wedge wings 720 in the raised position.

As shown in FIGS. 19, 24, and 25, the strap 740 includes a strap portion 742 (e.g., a strap body, an elastic strap) configured to wrap around a portion of the lower limb 50 of the patient and a pair of coupling portions 744 configured to releasably couple to the mounting portions 730 of the wedge pad 710 to couple the strap 740 to the wedge pad 710. For example, the coupling portions 744 may be the second portions of the hook-and-loop fasteners (e.g., the hook portions when the mounting portions 730 are configured as the loop portions, the loop portions when the mounting portions 730 are configured as the hook portions) configured to engage the mounting portions 730 configured as the first portions of the hook-and-loop fasteners to releasably couple the strap 740 to the wedge pad 710. As other examples, the coupling portions 744 may be a second portion of the button fastener (e.g., the male button when the mounting portions 730 are configured as female buttons, the female button when the mounting portions 730 are configured as male buttons), a second portion of the adhesive fastener (e.g., the adhesive portion when the mounting portions 730 are configured as the adhesive receiving portion, the adhesive receiving portion when the mounting portions 730 are configured as the adhesive portion), a second portion of the clip fastener (e.g., the male clip when the mounting portions 730 are configured as the female clip, the female clip when the mounting portions 730 are configured as the male clip), etc.

To couple the lower limb 50 of the patient to the wedge pad 710, a first of the coupling portions 744 may be coupled to a first of the mounting portions 730, the strap portion 742 may be wrapped around a top of the lower limb 50 of the patient such that the lower limb 50 of the patient is positioned between the strap portion 742 and the wedge pad 710, and a second of the coupling portions 744 may be coupled to a second of the mounting portions 730.

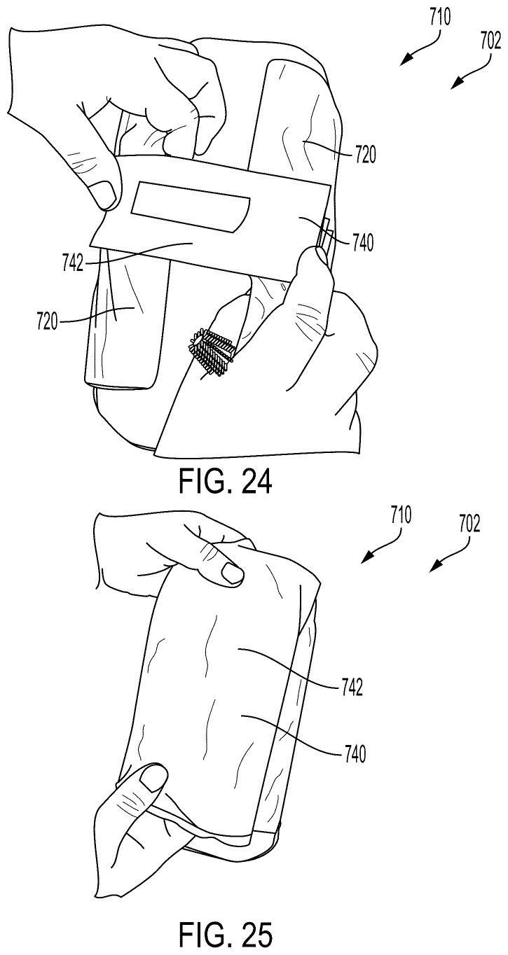

As shown in FIG. 24, when the mounting portions 730 are positioned on the wedge wings 720, a length of the wedge wings 720 between the wing tip edge 724 and the wing base edge 726 may be greater than a width of the strap 740 in a direction parallel to the length of the wedge wings 720, according to some embodiments. For example, when the strap 740 is coupled to each of the wedge wings 720, the strap 740 may cover a portion of a gap (e.g., an opening) between the wedge wings 720 such that the lower limb 50 of the patient may be accessible through other portions of the gap between the wedge wings 720. In other embodiments, as shown in FIG. 25, when the mounting portions are positioned on the wedge wings 720, the width of the strap 740 in the direction parallel to the length of the wedge wings 720 may be greater than or equal to the length of the wedge wings 720. For example, when the strap 740 is coupled to each of the wedge wings 720, the strap 740 may cover the gap between the wedge wings 720 such that the lower limb 50 of the patient cannot be accessed through the gap between the wedge wings 720.

In some embodiments, when the mounting portions 730 are positioned on the wedge side panel 716 of the wedge pad 710, a length of the wedge pad 710 from the wedge tip end 704 to the wedge base end 706 is greater than a width of the strap 740 in a direction parallel to the length of the wedge pad 710. For example, when the strap 740 is coupled to each of the wedge side panel 716, the strap 740 may cover a portion of the lower limb 50 of the patient supported by the wedge pad 710 such that other portions of the lower limb 50 of the patient may be accessible. In other embodiments, when the mounting portions 730 are positioned on the wedge side panel 716 of the wedge pad 710, the width of the strap 740 in the direction parallel to the length of the wedge pad 710 is greater than or equal to the length of the wedge pad 710.

In some embodiments, the strap portion 742 is formed from an elastic material so that as the strap portion 742 is stretched around the lower limb 50 of the patient to allow for the coupling portions 744 to be coupled to the mounting portions 730, the strap portion 742 applies a compression force on the lower limb 50 that is in a direction towards the wedge pad 710. The compression force may assist in securing the lower limb 50 to the wedge pad 710. For example, the strap portion 742 may be formed out of an elastic material such as spandex. The strap 740 may be a disposable element (e.g., an element that is disposed of after each use, an element that is disposed of after each patient) since the strap 740 may be in direct contact with the lower limb 50 of the patient when the strap 740 is securing the lower limb 50 to the wedge pad 710.

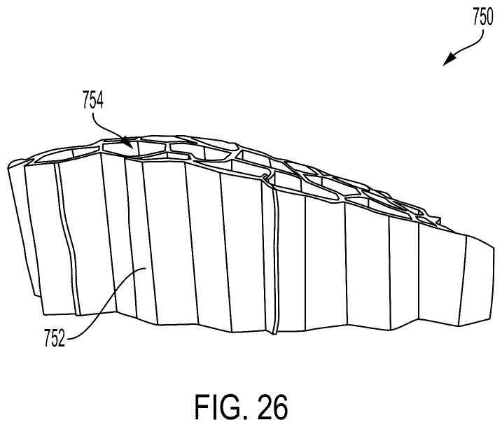

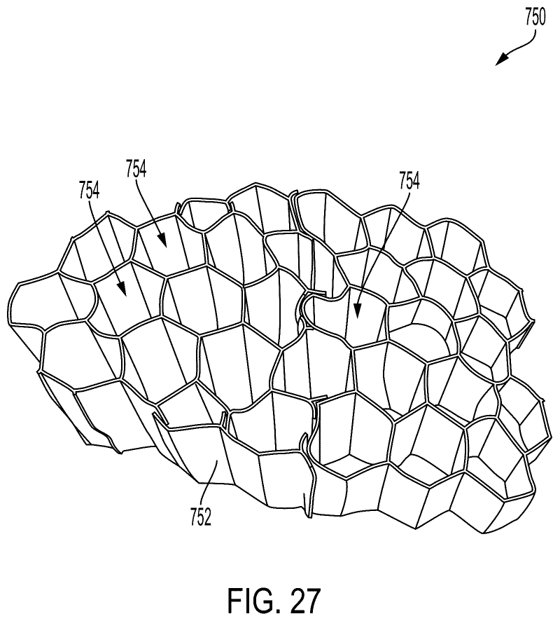

As shown in FIGS. 26 and 27, the wedge pad 710 includes a support gel matrix 750 configured to cushion and/or support the lower limb 50 of the patient, according to some embodiments. The support gel matrix 750 may be positioned within a cavity (e.g., an opening) of the wedge pad 710 defined by the wedge bottom panel 712, the wedge top panel 714, the wedge side panels 716, and/or the wedge base panel 718. In some embodiments, the support gel matrix 750 is formed to match a profile of the lower limb 50 of the patient. For example, the support gel matrix 750 may be molded to offload the heel portion 58 of the lower limb 50 of the patient and disperse the pressure created on the Achilles' tendon and calf of the patient to prevent the formation of pressure injures on the heel portion 58.

The support gel matrix 750 may include a gel structure 752 defining a plurality of matrix openings 754. For example, the gel structure 752 may be Stryker IsoTour support gel or another type of support gel. The configuration of the gel structure 752 and the matrix openings 754 may allow for pressure applied by the wedge pad 710 on the lower limb 50 to be evenly distributed along a portion of the lower limb 50 that contacts the wedge pad 710 when the lower limb 50 is supported by the wedge pad 710. Additionally or alternatively, the configuration of the gel structure 752 and the matrix openings 754 may allow for air to flow through the support gel matrix 750 such that the wedge pad 710 is breathable. When the wedge pad 710 is breathable, the temperature of the lower limb 50 of the patient supported by the wedge pad 710 may be regulated and moisture may be dissipated from the lower limb 50 through the wedge pad 710. In other embodiments, the wedge pad 710 includes another cushioning material (e.g., other than the support gel matrix 750) positioned within the cavity of the wedge pad 710 and configured to cushion and/or support the lower limb 50 of the patient. For example, the cushioning material may be formed out of a foam material such as memory foam. In various embodiments, the wedge wings 720 may include a support gel matrix or another cushioning material configured to cushion the lower limb 50 of the patient when the lower limb 50 contacts the wedge wings 720.

In some embodiments, the wedge cushioning system 700 is configured to support one of a left lower limb of the patient or a right lower limb of the patient. For example, a shape of the wedge pad 710 may be configured to specifically support the left lower limb of the patient or the right lower limb of the patient. Based on which of the lower limbs 50 the wedge cushioning system 700 is configured to support, the relative sizes of the components of the wedge pad 710 may change. For example, when the wedge cushioning system 700 is configured to support the left lower limb of the patient, the wedge side panel 716 on the left side of the wedge pad 710 may be shorter than the wedge side panel 716 on the right side of the wedge pad 710 such that the wedge top panel 714 is angled downward toward the left side of the wedge pad 710. As another example, when the wedge cushioning system 700 is configured to support the right lower limb of the patient, the wedge wing 720 on the right side of the wedge assembly 702 may be thicker (e.g., provide more cushioning) than the wedge wing 720 on the left side of the wedge assembly 702 since it may be more likely that the right lower limb of the patient contacts the wedge wing 720 on the right side of the wedge assembly 702 than the wedge wing 720 on the left side of the wedge assembly 702. In other embodiments, the wedge cushioning system 700 is configured as a universal wedge that can support either the left lower limb of the patient or the right lower limb of the patient. For example, the wedge cushioning system 700 may have a single configuration that may be used to support the right lower limb or the left lower limb of the patient with similar results.

As shown in FIGS. 19, 28, and 29, the heel wrap assembly 708 includes a heel wrap 760 (e.g., a foot wrap, a foot strap) configured to be wrapped around the heel portion 58 of the lower limb 50 of the patient and a gripping portion 770 configured to restrict movement of the lower limb 50 relative to the wedge assembly 702 when the lower limb 50 is supported by the wedge assembly 702 and the heel wrap assembly 708 is positioned on the lower limb 50, according to some embodiments. The heel wrap assembly 708 may be positioned between the heel portion 58 and the wedge pad 710 when the wedge pad 710 is supporting the lower limb 50 of the patient to prevent direct contact between the wedge pad 710 and the lower limb 50.

The heel wrap assembly 708 may be configured as a disposable component (e.g., a component that can be used for a single use, a component that can be used for a single patient of the wedge cushioning system 700) since the heel wrap assembly 708 will have direct contact with the lower limb 50 of the patient. In some embodiments, when the heel wrap assembly 708 is configured as a disposable component, the heel wrap assembly 708 may be formed and/or coated with materials that increase a comfort of the patient. For example, the heel wrap assembly 708 may be formed and/or coated with cotton, fleece, or other materials that may be gentile on the skin of the patient but may be hard to clean for reuse. In some embodiments, when the heel wrap assembly 708 is configured as a disposable component, the heel wrap assembly 708 may be formed and/or coated with materials that are fragile. For example, the heel wrap assembly 708 may be formed and/or coated with paper, thin cotton sheeting, or other materials that may rip or tear easily. When the heel wrap assembly 708 disposable, the wedge pad 710 and/or other components of the wedge assembly 702 may be configured as reusable components since the heel wrap assembly 708 limits direct contact between the lower limb 50 of the patient and the wedge pad 710. Specifically, the heel wrap assembly 708 may cover a pressure sore positioned on the heel portion 58 of the lower limb 50 of the patient to prevent direct contact between the wedge assembly 702 and the pressure sore, which could contaminate the wedge assembly 702 (e.g., contaminate with bacteria, contaminate with blood borne pathogens).

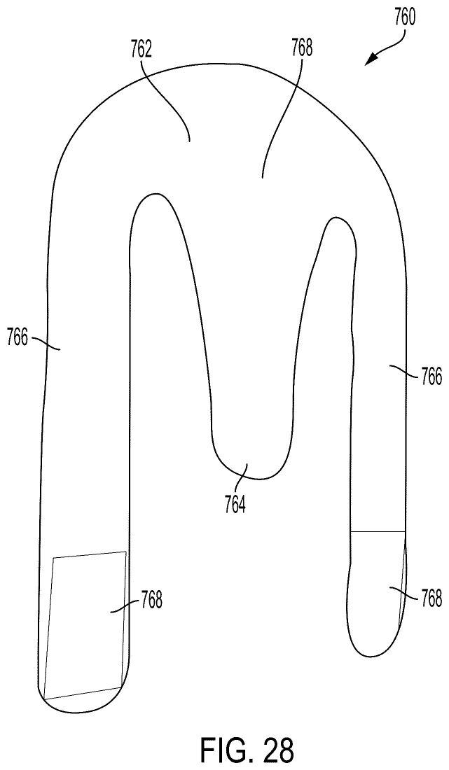

As shown in FIGS. 19 and 28, the heel wrap 760 includes a wrap base portion 762 (e.g., a wrap sole portion, a sole cover) configured to be positioned under a sole of the foot portion 56 of the lower limb 50 of the patient and a wrap heel portion 764 (e.g., a heel cover) extending from the wrap base portion 762 and configured to be positioned under the heel portion 58 of the foot portion 56 of the lower limb 50 of the patient. The wrap base portion 762 and the wrap heel portion 764 may be formed out of a breathable non-woven substrate. In some embodiments, the wrap base portion 762 and the wrap heel portion 764 are integrally formed. The wrap heel portion 764 may be configured to match a profile of the heel portion 58 of the lower limb 50. For example, a width of the wrap heel portion 764 may be substantially equal to a width of the heel portion 58. In some embodiments, the wrap heel portion 764 is also configured to be positioned under a calf portion of the lower limb 50. For example, when the heel wrap 760 is positioned under the lower limb 50, the wrap heel portion 764 may extend from the wrap base portion 762 under the heel portion 58 and under the calf portion above the heel portion 58.

As shown in FIGS. 19 and 28, the heel wrap 760 includes a pair of wrap arms 766 (e.g., attachment arms) extending from the wrap base portion 762 and configured to be wrapped around the lower limb 50 of the patient to couple the heel wrap 760 to the lower limb 50 of the patient. A first of the wrap arms 766 may be wrapped around the lower limb 50 in a first direction (e.g., clockwise) and a second of the wrap arms 766 may be wrapped around the lower limb 50 in a second direction (e.g., counter clockwise) such that the wrap arms 766 are positioned on opposing sides of the lower limb 50 to couple the heel wrap 760 to the lower limb 50. The first of the wrap arms 766 may overlap the second of the wrap arms 766 when the wrap arms 766 are wrapped around the lower limb 50 of the patient. The wrap arms 766 may be formed out of a breathable non-woven substrate. In some embodiments, the wrap arms 766 and the wrap base portion 762 are integrally formed (e.g., formed out of a single piece of breathable non-woven substrate).

As shown in FIGS. 19 and 28, the heel wrap 760 includes adhesive portions 768 configured to couple the heel wrap 760 to the lower limb 50 of the patient, according to some embodiments. The adhesive portions 768 may be laminated to the other components of the heel wrap 760. A first of the adhesive portions 768 may be positioned on a first surface of the wrap base portion 762 and the wrap heel portion 764 and be configured to adhere the heel wrap 760 to the sole of the foot portion 56 of the lower limb 50 and to the heel portion 58 of the lower limb 50. For example, the first of the adhesive portions 768 may be configured to adhere the heel wrap 760 to an Achilles' tendon area of the lower limb 50. In some embodiments, the first of the adhesive portions 768 may be configured to adhere the heel wrap 760 to the calf portion of the lower limb 50 (e.g., when the wrap heel portion 764 extends under the calf portion). A second and a third of the adhesive portions 768 may be positioned at free ends of the wrap arms 766 to couple the free ends of the wrap arms 766 to the lower limb 50 and/or to the wrap base portion 762 when the wrap arms 766 are wrapped around the lower limb 50. In other embodiments, the heel wrap 760 is coupled to the lower limb 50 using other means (e.g., button fasteners, clip fasteners, hook and loop fasteners).

In some embodiments, the second and the third of the adhesive portions 768 are configured to adhere the wrap arms 766 to the lower limb 50 when the wrap arms 766 are wrapped around the lower limb 50 to couple the heel wrap 760 to the lower limb 50. In other embodiments, the second and the third of the adhesive portions 768 are configured to adhere the wrap arms 766 to other components of the heel wrap 760 to couple the free ends of the wrap arms 766 to the lower limb 50 and couple the heel wrap 760 to the lower limb 50. For example, the second of the adhesive portions 768 positioned on a first of the wrap arms 766 may be configured to adhere to a second of the wrap arms 766 when the first of the wrap arms 766 is wrapped around the lower limb 50 and the third of the adhesive portions 768 positioned on the second of the wrap arms 766 may be configured to adhere to the first of the wrap arms 766 when the second of the wrap arms 766 is wrapped around the lower limb 50. As another example, the second and the third of the adhesive portions 768 positioned on the wrap arms 766 may be configured to adhere to the wrap base portion 762 and/or the wrap heel portion 764 when the wrap arms 766 are wrapped around the lower limb 50 to couple the heel wrap 760 to the lower limb 50.

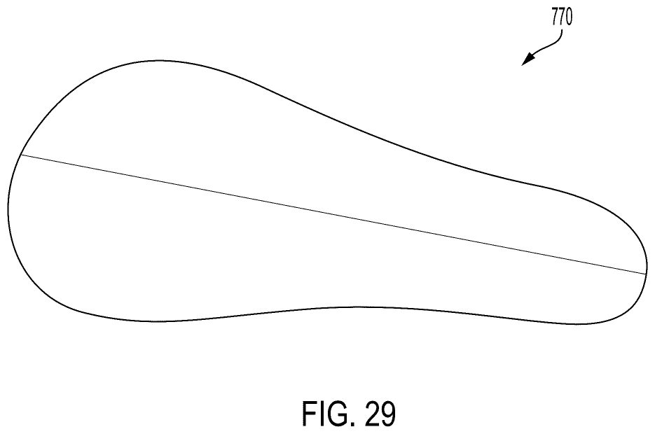

As shown in FIGS. 19 and 29, the gripping portion 770 is configured to be positioned on a second surface of the wrap base portion 762 and the wrap heel portion 764 opposing the first surface (e.g., the first surface where the first of the adhesive portions 768 is positioned) such that the heel wrap 760 is positioned between the gripping portion 770 and the lower limb 50 when the heel wrap assembly 708 is positioned on the lower limb 50, according to some embodiments. The gripping portion 770 may be laminated to the wrap base portion 762 and the wrap heel portion 764. In some embodiments, the gripping portion 770 is formed and/or coated with a material that has a high coefficient of friction (e.g., higher than the surface under the patient, higher than the heel wrap 760, etc.). For example, the gripping portion 770 may be formed and/or coated with silicone rubber, neoprene, polyurethane foam, or other materials with coefficients of friction that are higher than a coefficient of friction of the heel wrap 760. When the heel wrap assembly 708 is positioned on the lower limb 50 of the patient and the lower limb 50 is supported by the wedge pad 710, the gripping portion 770 with the high coefficient of friction may contact the wedge top panel 714 with the high coefficient of friction. The friction between the gripping portion 770 and the wedge top panel 714 may restrict the movement of the lower limb 50 relative to the wedge pad 710 to prevent the lower limb 50 from moving relative to the wedge pad 710 to a non-ideal orientation.

### Overview of Dressing Boot Assembly

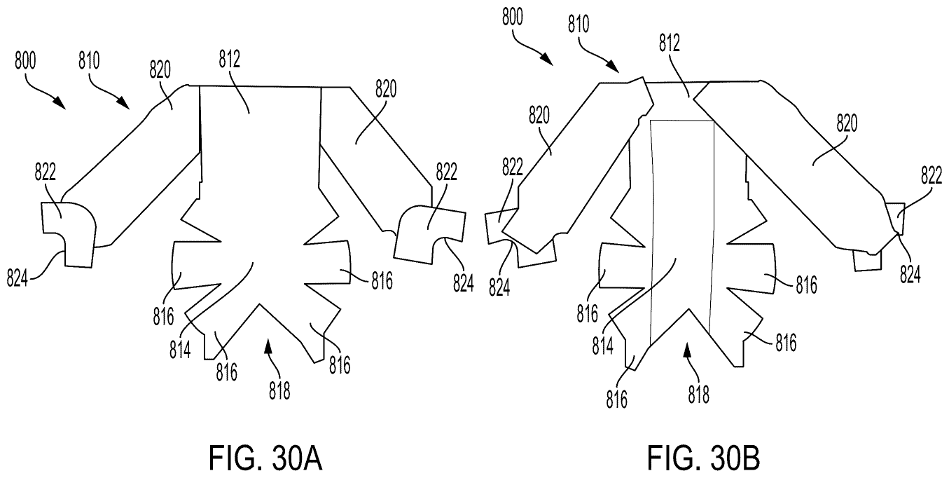

As shown in FIGS. 30A-33D, the lower limb positioning system 10 includes a dressing boot assembly 800 (e.g., a dressing boot, a dressing boot system) configured to cushion and/or support the heel portion 58 of the lower limbs 50 of patients, according to some embodiments. For example, when a patient is positioned on a bed the dressing boot assembly 800 may be positioned on the lower limb 50 of the patient to offload the heel portion 58 of the lower limb 50 of the patient from a surface of the bed. In some embodiments, the materials used in the dressing boot assembly 800 are breathable and are intended to keep the microclimate of the lower limb 50 cool and dry. For example, a high suction capillary action foam may be used to keep the microclimate of the lower limb 50 dry. When the dressing boot assembly 800 is used to cushion and/or support the heel portion 58, the dressing boot assembly 800 may prevent pressure injuries to the heel portion 58 of the lower limb 50 of the patient. Additionally, the dressing boot assembly 800 may prevent dropping of the foot portion 56 of the lower limb 50 (e.g., tilting forward of a front of the foot portion 56 so that toes of the foot portion 56 drag on the ground) and/or rolling out of the foot portion 56 of the lower limb 50 when the patient is mobile, which can both cause issues with walking when the patient is mobile.

In some embodiments, the dressing boot assembly 800 is configured as a disposable element (e.g., an element that can be used for a single use, an element that can be used for a single patient of the dressing boot assembly 800) since the dressing boot assembly 800 will have direct contact with the lower limb 50 of the patient. In other embodiments, the dressing boot assembly 800 is configured as a reusable element (e.g., an element that can be used for multiple patients). For example, the dressing boot assembly 800 may be formed and/or coated with materials that are easy to clean, materials that have antiseptic properties, and/or materials with a high durability.

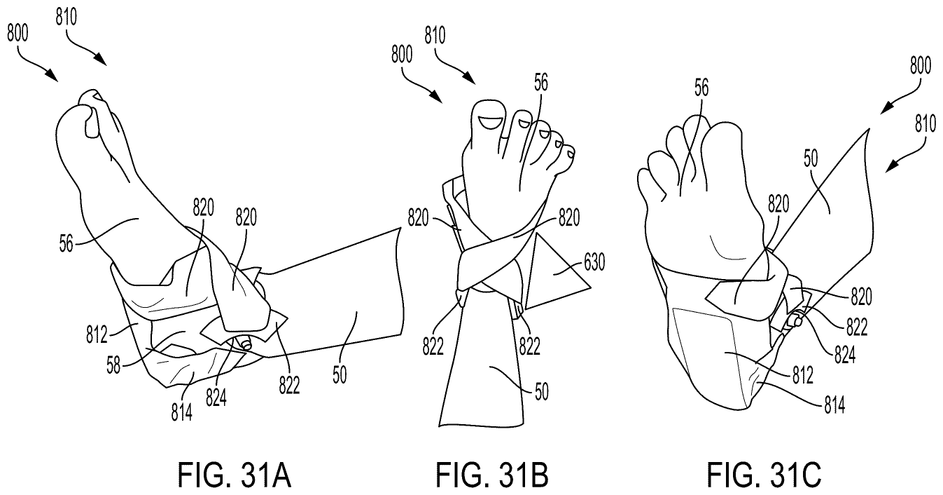

As shown in FIGS. 30A-31C, the dressing boot assembly 800 is configured as a star dressing assembly 810 configured to cushion and/or support the heel portion 58 of the lower limb 50 of the patient, according to some embodiments. The star dressing assembly 810 includes a portion that is star-shaped and that is configured to wrap around the foot portion 56 of the lower limb 50 of the patient. The star dressing assembly 810 may include a cutout that aligns with an Achilles tendon of the lower limb 50 to relieve pressure on the Achilles tendon. In other embodiments, the dressing boot assembly 800 is configured as a cross dressing assembly that includes a portion that is cross-shaped and is configured to wrap around the foot portion 56 of the patient. In still other embodiments, the dressing boot assembly 800 is configured as a circular dressing assembly that includes a portion that is circle shaped and is configured to wrap around the foot portion 56 of the patient. The portion that is circle shaped may include cuts extending radially from an outside of the portion toward a center of the circle shape to allow for the portion to be wrapped around the foot portion 56 of the patient without bunching up.

As shown in FIGS. 30A-31C, the star dressing assembly 810 includes a dressing base portion 812 (e.g., a dressing sole portion, a sole cover) configured to be positioned under the sole of the foot portion 56 of the lower limb 50 of the patient and a star heel portion 814 configured to be positioned under the heel portion 58 of the foot portion 56 of the lower limb 50 of the patient. The dressing base portion 812 and the star heel portion 814 may be formed from a dual layered perforated silicone and absorbable foam pad configured to regulate a moisture of the foot portion 56 while the star dressing assembly 810 is positioned on the lower limb 50. The star heel portion 814 includes a plurality of wrap extensions 816 separated by openings configured to wrap around the heel portion 58 of the patient. For example, each of the wrap extensions 816 may be separately formed around the heel portion 58 to allow for the wrap extensions 816 to fully contact the heel portion 58 without portions of the star heel portion 814 bunching up around the heel portion 58.

The star heel portion 814 defines an Achilles opening 818 configured to align with the Achilles tendon of the foot portion 56 such that the star dressing assembly 810 applies minimal pressure on the Achilles tendon when the star dressing assembly 810 is positioned on the lower limb 50 while still protecting the heel portion 58 from pressure injury. The Achilles opening 818 may be defined between two of the wrap extensions 816 such that the two of the wrap extensions 816 are positioned on opposing sides of the Achilles tendon when the star dressing assembly 810 is positioned on the lower limb 50. The star heel portion 814 and/or the dressing base portion 812 may include an adhesive portion configured to adhere the star heel portion 814 and/or the dressing base portion 812 to the lower limb 50. For example, an adhesive portion may be positioned at the ends of each of the wrap extensions 816 to adhere the wrap extensions 816 to the heel portion 58 when the wrap extensions 816 are wrapped around the heel portion 58.

As shown in FIGS. 30A-31C, the star dressing assembly 810 includes a pair of dressing arms 820 (e.g., attachment arms, wrap arms) extending from the dressing base portion 812 and configured to be wrapped around the lower limb 50 of the patient to couple the star dressing assembly 810 to the lower limb 50 of the patient. A first of the dressing arms 820 may be wrapped around the lower limb 50 in a first direction and a second of the dressing arms 820 may be wrapped around the lower limb 50 in a second direction such that the dressing arms 820 are positioned on opposing sides of the lower limb 50 to couple the star dressing assembly 810 to the lower limb 50. The first of the dressing arms 820 may overlap the second of the dressing arms 820 when the dressing arms 820 are wrapped around the lower limb 50 of the patient. The dressing arms 820 may be formed from an elastic material (e.g., a stretchy material, spandex) to prevent the foot portion 56 of the lower limb 50 from dropping forward when the star dressing assembly 810 is positioned on the lower limb 50 of the patient. For example, when the patient is attempting to walk, the patient may not have an ability (e.g., due to a lack of strength, due to a lack of coordination) to prevent the foot portion 56 from dropping forward and dragging on the ground. The elasticity of the dressing arms 820 may provide an elastic restoring force on the foot portion 56 (e.g., via the dressing base portion 812) to prevent the foot portion 56 from dropping forward.

As shown in FIGS. 30A-31C, the star dressing assembly 810 includes adhesive pads 822 (e.g., adhesive portions) positioned at ends of the dressing arms 820 to couple the dressing arms 820 to the lower limb 50 when the dressing arms 820 are wrapped around the lower limb 50. For example, a first of the adhesive pads 822 may be positioned at an end of the first of the dressing arms 820 to adhere the first of the dressing arms 820 to the lower limb 50 and a second of the adhesive pads 822 may be positioned at an end of the second of the dressing arms 820 to adhere the second of the dressing arms 820 to the lower limb 50. Each of the adhesive pads 822 defines a locating aperture 824 configured to be positioned around a bone extending out from the ankle portion 60. The locating aperture 824 may allow for a caregiver to adhere the adhesive pads 822 quickly and easily in a specific position (e.g., a correct position, an intended position) relative to the lower limb 50. For example, in order for the dressing arms 820 to prevent foot drop, the dressing arms 820 may need to be placed in tension when the star dressing assembly 810 is positioned on the lower limb 50. By aligning the locating aperture around the bone extending out from the ankle portion 60 when positioning the star dressing assembly 810 on the lower limb 50, an appropriate amount of pre-tension may be applied on the dressing arms 820 to lift the foot portion 56 and prevent the foot portion 56 from dropping.

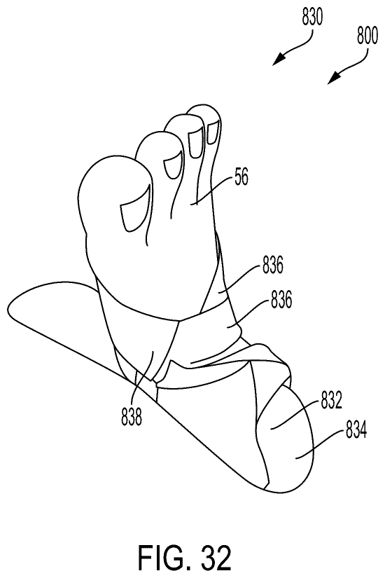

As shown in FIG. 32, the dressing boot assembly 800 is configured as a soleless dressing assembly 830 configured to cushion and/or support the heel portion 58 of the lower limb 50 of the patient, according to some embodiments. The soleless dressing assembly 830 is configured to wrap around the foot portion 56 of the lower limb 50 while covering a minimal area of the sole of the foot portion 56 to assist with temperature regulation of the foot portion 56.

As shown in FIG. 32, the soleless dressing assembly 830 includes a dressing base 832 configured to be positioned under the heel portion 58 of the foot portion 56 of the lower limb 50 of the patient. The dressing base 832 may be formed from a dual layered perforated silicone and absorbable foam pad configured to regulate a moisture of the foot portion 56 when the soleless dressing assembly 830 is positioned on the lower limb 50. The dressing base 832 may be configured to match a profile of the lower limb 50. For example, the dressing base 832 may be configured to match the profile of the heel portion 58 to appropriately support the heel portion 58 when the soleless dressing assembly 830 is positioned on the lower limb 50.

As shown in FIG. 32, the dressing base 832 includes labels 834 (e.g., written labels, illustrative labels) that show how the dressing base 832 should be oriented relative to the lower limb 50 when the soleless dressing assembly 830 is positioned on the lower limb 50, according to some embodiments. For example, the dressing base 832 may include the label 834 of “ANKLE” on a portion of the dressing base 832 that should be positioned under the ankle portion 60 of the lower limb 50 when the soleless dressing assembly 830 is positioned on the lower limb 50. As another example, the dressing base 832 may include the label 834 of an illustration indicating a heel on a portion of the dressing base 832 that should be positioned under the heel portion 58 of the lower limb 50. In some embodiments, the label 834 may be positioned on an adhesive portion (e.g., an adhesive strip) of the dressing base 832 and the label 834 may indicate where on the lower limb 50 to adhere the adhesive portion of the dressing base 832.

In some embodiments, the soleless dressing assembly 830 may be configured as a universal dressing assembly configured to be positioned on a plurality of different sizes of the lower limbs 50. A length of the dressing base 832 may be configured to extend down past the sole of the foot portion 56 when the soleless dressing assembly 830 is positioned on most sizes of the lower limbs 50 (e.g., 99% percentile of lower limbs 50) so that the dressing base 832 is positioned between the heel portion 58 and the surface below the patient for a majority of the lower limbs 50. In other embodiments, different configurations (e.g., a small configuration, a medium configuration, a large configuration) of the soleless dressing assembly 830 are configured to be positioned on specific sizes of the lower limb 50.

As shown in FIG. 32, the soleless dressing assembly 830 includes a pair of dressing arms 836 (e.g., attachment arms, wrap arms) extending from the dressing base 832 and configured to be wrapped around the foot portion 56 of the lower limb 50 of the patient to couple the soleless dressing assembly 830 to the lower limb 50 of the patient. A first of the dressing arms 836 may be wrapped around the foot portion 56 of the lower limb 50 in a first direction and a second of the dressing arms 836 may be wrapped around the foot portion 56 of the lower limb 50 in a second direction such that the dressing arms 836 are positioned on opposing sides of the foot portion 56 to couple the soleless dressing assembly 830 to the lower limb 50. The first of the dressing arms 836 may overlap the second of the dressing arms 836 when the dressing arms 836 are wrapped around the foot portion 56 of the lower limb 50 of the patient. The dressing arms 836 may be formed from an elastic material (e.g., a stretchy material, spandex) to prevent the foot portion 56 of the lower limb 50 from dropping forward when the soleless dressing assembly 830 is positioned on the lower limb 50 of the patient. For example, when the patient is attempting to walk, the patient may not have an ability (e.g., due to a lack of strength, due to a lack of coordination) to prevent the foot portion 56 from dropping forward and dragging on the ground. The elasticity of the dressing arms 836 may provide an elastic restoring force on the foot portion 56 to prevent the foot portion 56 from dropping forward.

As shown in FIG. 32, the soleless dressing assembly 830 includes side adhesive pads 838 (e.g., side adhesive portions) positioned at ends of the dressing arms 836 to couple the dressing arms 836 to the foot portion 56 when the dressing arms 836 are wrapped around the foot portion 56. For example, a first of the side adhesive pads 838 may be positioned at an end of the first of the dressing arms 836 to adhere the first of the dressing arms 836 to the foot portion 56 and a second of the side adhesive pads 838 may be positioned at an end of the second of the dressing arms 836 to adhere the second of the dressing arms 836 to the foot portion 56. The side adhesive pads 838 may be configured to adhere to an arch of the foot portion 56. For example, the side adhesive pads 838 may be curved to match a profile of the arch of the foot portion 56 to adhere to the arch of the foot portion.

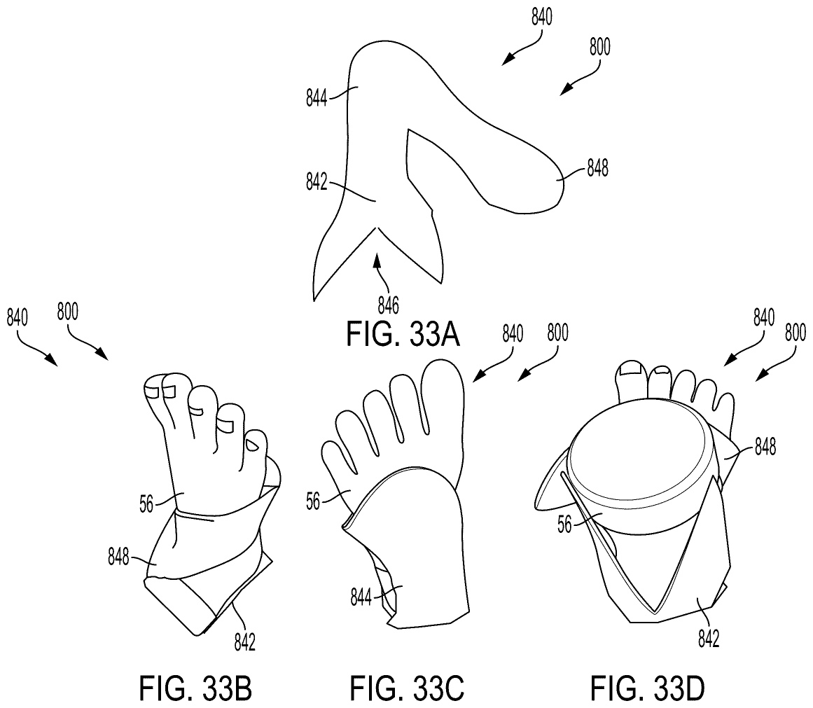

As shown in FIGS. 33A-33D, the dressing boot assembly 800 is configured as an integrated dressing boot 840 configured to cushion and/or support the heel portion 58 of the lower limb 50 of the patient, according to some embodiments. The integrated dressing boot 840 may be formed from a single element of dual layered perforated silicone and absorbable foam pad configured to regulate moisture of the foot portion 56 when the integrated dressing boot 840 is positioned on the lower limb 50 of the patient.

As shown in FIGS. 33A-33D, the integrated dressing boot 840 includes a dressing heel portion 842 configured to be positioned under the heel portion 58 of the foot portion 56 and a dressing sole portion 844 extending from the dressing heel portion 842 and configured to be positioned under the sole of the foot portion 56. The dressing heel portion 842 and the dressing sole portion 844 may be integrally formed (e.g., formed from a single piece of material). The dressing heel portion 842 defines an Achilles opening 846 configured to align with the Achilles tendon of the foot portion 56 such that the integrated dressing boot 840 applies minimal pressure on the Achilles tendon when the integrated dressing boot 840 is positioned on the lower limb 50 while still protecting the heel portion 58 from pressure injury. The dressing heel portion 842 and/or the dressing sole portion 844 may include an adhesive portion configured to adhere the dressing heel portion 842 and/or the dressing sole portion 844 to the lower limb 50. For example, an adhesive portion may be positioned on the dressing heel portion 842 to adhere the dressing heel portions 842 on either side of the Achilles opening 846 when the integrated dressing boot 840 is positioned on the lower limb 50.

As shown in FIGS. 33A-33D, the integrated dressing boot 840 includes a dressing wrap portion 848 extending from the dressing sole portion 844 and configured to be wrapped around the lower limb 50 of the patient to couple the integrated dressing boot 840 to the lower limb 50. The dressing sole portion 844 and the dressing wrap portion 848 may be integrally formed. In some embodiments, the dressing wrap portion 848 and the dressing heel portion 842 may form an acute angle when extending from the dressing sole portion 844. For example, the dressing wrap portion 848 and the dressing heel portion 842 may extend from the dressing sole portion 844 at a sixty degree angle from each other. In other embodiments, the dressing wrap portion 848 and the dressing heel portion 842 may form other angles (e.g., a perpendicular angle, an obtuse angle) when extending from the dressing sole portion 844.

A shape of the dressing wrap portion 848 may be configured to match a profile of the lower limb 50. For example, the shape of the dressing wrap portion 848 may be a bowtie to allow for the dressing wrap portion 848 to match a profile between the foot portion 56 and the ankle portion 60. The dressing wrap portion 848 may include an adhesive portion configured to adhere the dressing wrap portion 848 to the lower limb 50. For example, an end of the dressing wrap portion 848 may include an adhesive strip configured to adhere to the ankle portion 60 of the lower limb 50 to couple the integrated dressing boot 840 to the lower limb 50.

As shown in FIG. 31B, the dressing boot assembly 800 may be held in position by wedge modules 630. For example, as shown in FIG. 31B, when the star dressing assembly 810 is positioned on the lower limb 50, the wedge module 630 may be edged under the foot portion 56 of the lower limb 50 to restrict movement of the foot portion 56 relative to the surface where the patient is positioned. When the dressing boot assembly 800 is positioned on the lower limb 50, the wedge modules 630 may be placed on either side of the dressing boot assembly 800 to restrict the movement of the dressing boot assembly 800 and the lower limb 50. Since the dressing boot assembly 800 may prevent some direct contact between the lower limb 50 and the wedge modules 630, the dressing boot assembly 800 may allow for the wedge modules 630 to be reusable while the dressing boot assembly 800 may be disposable.

The dressing boot assembly 800 may be used with components of the wedge cushioning system 700 to cushion and/or support the heel portion 58 of the lower limb 50 of the patient (e.g., in place of the heel wrap 760, in conjunction with the heel wrap 760). For example, the dressing boot assembly 800 may be positioned on the lower limb 50 of the patient and then the lower limb 50 may be positioned on the wedge assembly 702 such that the lower limb 50 is supported by the wedge assembly 702 and direct contact between the wedge assembly 702 and the lower limb 50 is prevented by the dressing boot assembly 800.

### Overview of Foot Drop Assembly

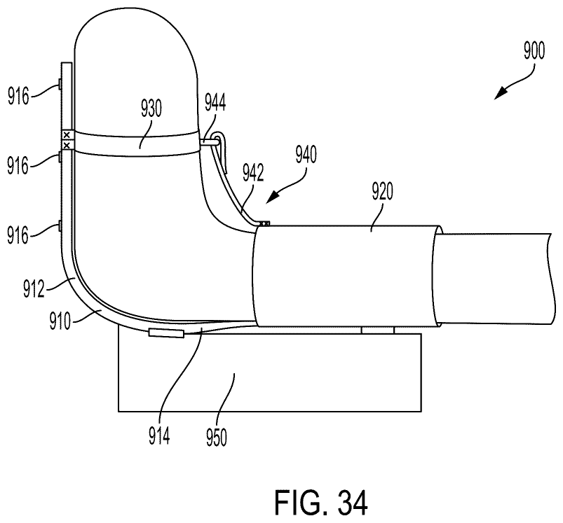

As shown in FIGS. 34-42, the lower limb positioning system 10 includes a foot drop assembly 900 (e.g., a foot drop boot, an elastic boot) configured to be positioned on the lower limb 50 of a patient to prevent dropping of the foot portion 56 of the lower limb 50. Additionally, the foot drop assembly 900 may cushion and/or support the heel portion 58 of the lower limb 50 of the patient to prevent pressure injuries from developing on the heel portion 58. As a result, the foot drop assembly 900 may be positioned on the lower limb 50 of the patient while the patient is lying in bed to prevent pressure injuries and be positioned on the lower limb 50 of the patient while the patient is ambulating to prevent the foot portion 56 of the lower limb 50 of the patient from dropping during ambulation without being removed between the two activities. Additionally, the foot drop assembly 900 may include traction features configured to provide traction to the patient during ambulation.

In some embodiments, the materials used to form components of the foot drop assembly 900 may have elastic properties. The elasticity of the components of the foot drop assembly 900 may prevent the dropping of the foot portion 56 by pulling the foot portion 56 upward. Additionally, the elasticity of the components of the foot drop assembly 900 may allow for changes in size of the lower limb 50 of the patient without causing discomfort. For example, the size of the lower limb 50 of the patient may change sizes significantly during a course of a stay of the patient. The elasticity of the components of the foot drop assembly 900 may allow for the foot drop assembly 900 to remain positioned on the lower limb 50 throughout the duration of the stay of the patient without causing discomfort to the patient. In some embodiments, the materials used in the foot drop assembly 900 are breathable and are configured to keep the microclimate of the lower limb 50 cool and dry.

As shown in FIGS. 34-42 the foot drop assembly 900 includes a foot support 910 configured to be positioned under the foot portion 56 of the lower limb 50 of the patient. The foot support 910 includes a sole support portion 912 configured to be positioned under the sole of the foot portion 56 of the lower limb 50 of the patient and a heel support portion 914 configured to be positioned under the heel portion 58 of the foot portion 56 of the lower limb 50 of the patient. The sole support portion 912 and/or the heel support portion 914 may be formed from a breathable material (e.g., a dual layered perforated silicone and absorbable foam pad) configured to the microclimate of the foot portion 56 of the lower limb 50 cool and dry.

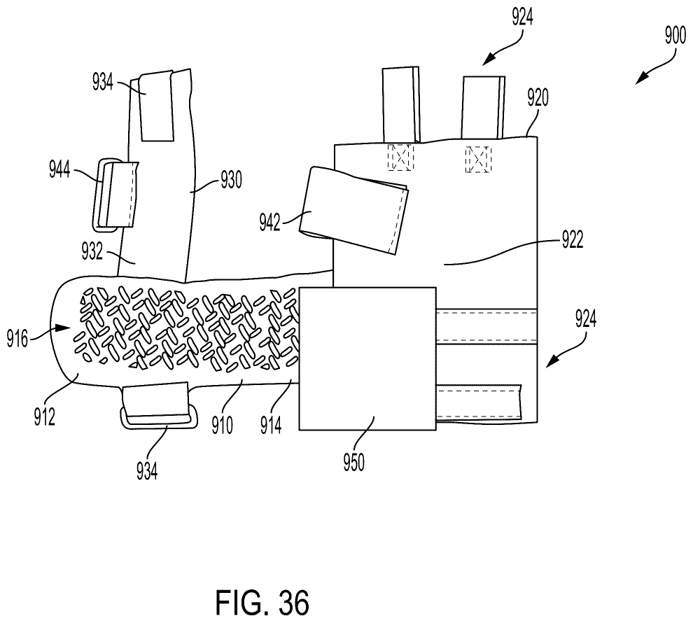

As shown in FIGS. 34, 36, 37, 39, 40, and 42, the foot support 910 includes a plurality of traction features 916 positioned on a bottom surface of the sole support portion 912 configured to provide traction between a surface where a patient is walking and the patient, according to some embodiments. For example, the traction features 916 may be extrusions extending downward from the bottom surface of the sole support portion 912 and may be formed and/or coated with a material with a high coefficient of friction (e.g., higher than the surface where the patient is walking, etc.) to provide the traction between the surface and the patient. For example, the traction features 916 may be formed and/or coated with silicone rubber, neoprene, polyurethane foam, or other materials with coefficients of friction that are higher than a standard floor of a hospital environment (e.g., a tile floor, a linoleum floor). The traction features 916 may be oriented on the bottom surface of the sole support portion 912 in patterns that promote high traction. For example, as shown in FIG. 36, the traction features 916 may be oriented in a cross hatch pattern. In other embodiments, the bottom surface of the sole support portion 912 is configured to provide traction between the surface where the patient is walking and the patient through other means (e.g., being formed from a material with a high coefficient of friction, being coated with a material with a high coefficient of friction).

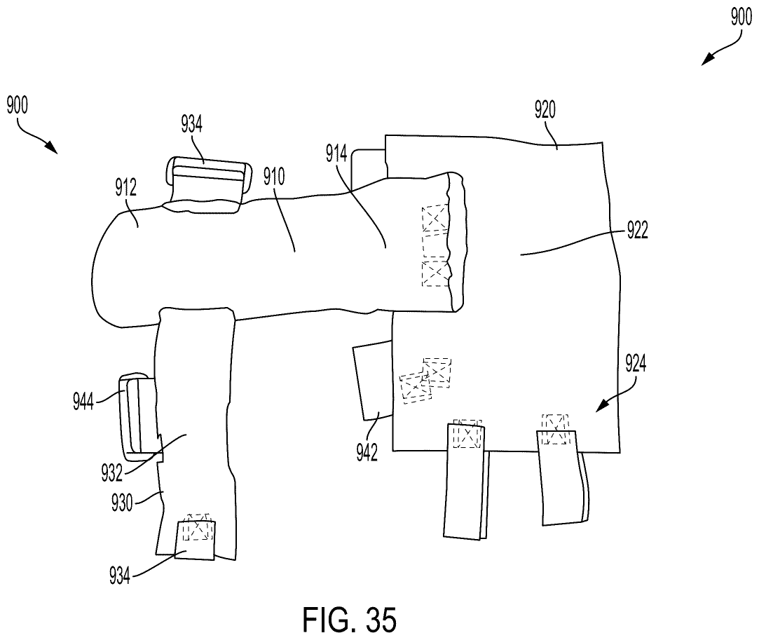

As shown in FIGS. 34-42, the foot drop assembly 900 includes a leg wrap 920 coupled to the heel support portion 914 of the foot support 910 and configured to be wrapped around the leg portion 52 of the lower limb 50 of the patient to couple the foot drop assembly 900 to the lower limb 50. For example, the leg wrap 920 may be configured to be wrapped around the leg portion 52 above the heel portion 58 to couple the foot drop assembly 900 to the lower limb 50. As shown in FIGS. 35-42, the leg wrap 920 includes a leg wrap portion 922 configured to be wrapped around the leg portion 52 of the lower limb 50 of the patient. The leg wrap portion 922 may be formed from an elastic material to allow for changes in the size of the leg portion 52 of the lower limb 50 of the patient while the foot drop assembly 900 is positioned on the lower limb 50 of the patient. For example, when the foot drop assembly 900 is positioned on the lower limb 50 of the patient, the leg portion 52 may have a first diameter. As conditions of the patient change, the leg portion 52 may swell up (e.g., expand) to a second diameter that is bigger than the first diameter. The elasticity of the leg wrap portion 922 may allow for the leg wrap portion 922 to be stretched when the leg portion 52 swells from the first diameter to the second diameter while still securing the foot drop assembly 900 to the lower limb 50. In some embodiments, the leg wrap 920 has a restorative force of less than approximately 28.25 newton millimeter (N*mm) (e.g., 0.25 lbs/inch) displaced, and is greater than approximately 38.1 mm (e.g., 1.5″) long, which ensures that it does not apply too much pressure onto the leg portion 52 of the lower limb 50 of the patient which could cause injury while still allowing for the foot drop assembly 900 to prevent the foot drop of the foot portion 56.

As shown in FIGS. 35-42, the leg wrap 920 includes a plurality of attachment portions 924 positioned on opposing ends of the leg wrap portion 922. The attachment portions 924 positioned on a first end of the leg wrap portion 922 are configured to releasably couple with the attachment portions on an opposing second end of the leg wrap portion 922 when the leg wrap portion 922 is wrapped around the leg portion 52 to couple the leg wrap 920 to the leg wrap portion 922. For example, a first of the attachment portions 924 positioned on the first end of the leg wrap portion 922 may be configured as a first portion of a hook-and-loop fastener (e.g., a hook portion, a loop portion) configured to engage a second of the attachment portions 924 positioned on the opposing second end of the leg wrap portion 922 that is configured as a second portion of the hook-and-loop fastener (e.g., the loop portion when the first of the attachment portions 924 is configured as the hook portion, the hook portion when the first of the attachment portions 924 is configured as the loop portion). As other examples, the attachment portions 924 may be portions of button fasteners, portions of adhesive fasteners, portions of clip fasteners, etc.

In some embodiments, the attachment portions 924 are configured as adjustable attachment portions configured to releasably couple to each other in different configurations. The adjustable attachment portions may allow for a compression applied by the leg wrap portion 922 on the leg portion 52 of the lower limb 50 to be changed based on the configuration of the adjustable attachment portions. For example, in a first configuration of the adjustable attachment portions, the leg wrap portion 922 may apply a first compression on the leg portion 52 of the lower limb 50 and in a second configuration of the adjustable attachment portions, the leg wrap portion 922 may apply a second compression on the leg portion 52 of the lower limb 50. The compression applied by the leg wrap portion 922 on the leg portion 52 of the lower limb 50 may be adjusted to promote blood flow through the lower limb 50 and/or protect the lower limb 50 of the patient from bruising. In other embodiments, the attachment portions 924 are configured as fixed attachment portions configured to releasably couple to each other in a single configuration.

As shown in FIGS. 34-42, the foot drop assembly 900 includes a foot wrap 930 coupled to the sole support portion 912 of the foot support 910 and configured to be wrapped around the foot portion 56 of the lower limb 50 of the patient to couple the foot drop assembly 900 to the lower limb 50. For example, the foot wrap 930 may be configured to be wrapped around the foot portion 56 distal from the heel portion 58 to couple the foot drop assembly 900 to the lower limb 50. As shown in FIGS. 34-42, the foot wrap 930 includes a foot strap 932 configured to be wrapped around the foot portion 56 of the lower limb 50 of the patient to couple the foot drop assembly 900 to the foot portion 56 of the lower limb 50 if the patient. The foot strap 932 may be formed from an elastic material to allow for changes in the size of the foot portion 56 if the lower limb 50 of the patient while the foot drop assembly 900 is positioned on the lower limb 50 of the patient.

As shown in FIGS. 34-42, the foot wrap 930 includes a plurality of attachment portions 934 configured to couple the foot strap 932 to the foot portion 56 of the lower limb 50. A first of the attachment portions 934 may be coupled to an end of the foot strap 932 (e.g., a free end of the foot strap 932). and a second of the attachment portions 934 may be coupled to the sole support portion 912. When the foot strap 932 is wrapped around the foot portion 56, the first of the attachment portions 934 may be releasably coupled with the second of the attachment portions 934 to secure the foot strap 932 around the foot portion 56. For example, the first of the attachment portions 934 may be configured as a self-coupling hook and loop fastener and the second of the attachment portions 934 may be configured as a buckle. When the foot strap 932 is wrapped around the foot portion 56, the first of the attachment portions 934 may be fed through the second of the attachment portions 934 and then releasably coupled to itself to releasably coupled the first of the attachment portions 934 to the second of the attachment portions 934 so that the foot strap 932 is secured around the foot portion 56 to couple the foot drop assembly 900 to the lower limb 50. As other examples, the attachment portions 934 may be portions of button fasteners, portions of hook-and-loop fasteners, portions of adhesive fasteners, portions of clip fasteners, etc.

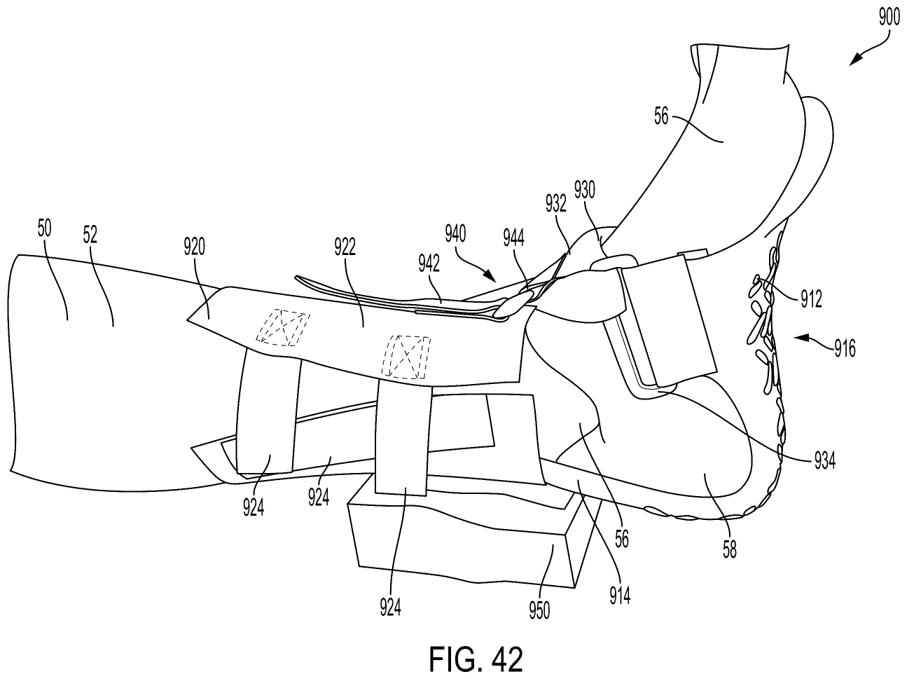

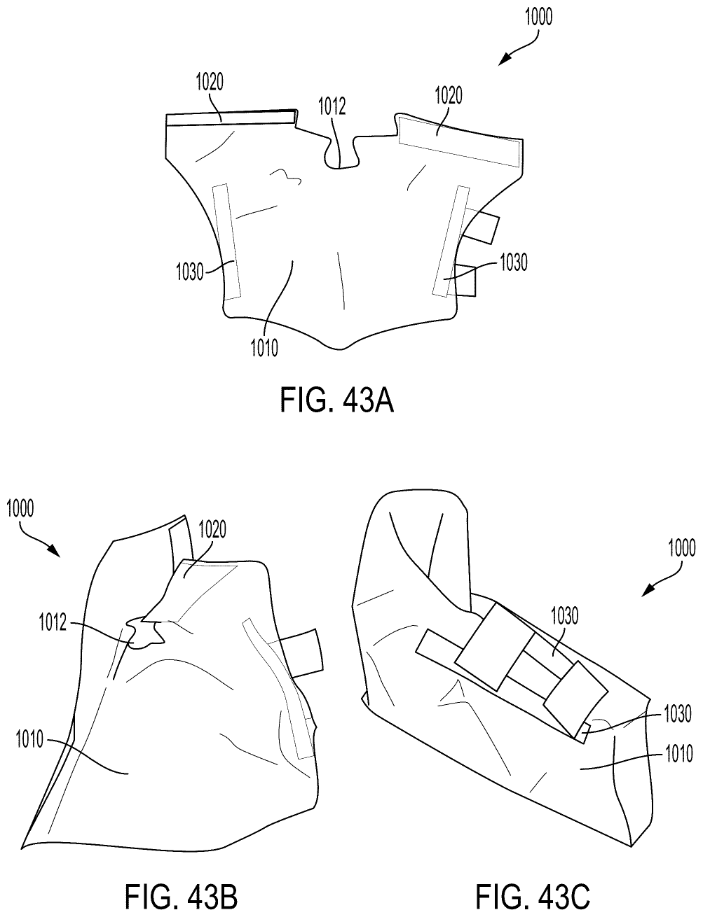

As shown in FIGS. 34-42, the foot drop assembly 900 includes a foot drop strap assembly 940 coupled to the leg wrap 920 and the foot wrap 930 and configured to prevent the foot portion 56 of the lower limb 50 from dropping forward (e.g., angling forward relative to the leg portion 52) during ambulation by the patient. As shown in FIGS. 43-42, the foot drop strap assembly 940 includes a foot drop strap 942 coupled to the leg wrap 920 and a foot drop buckle 944 coupled to the foot wrap 930, according to some embodiments. The foot drop strap 942 is configured to releasably couple to the foot drop buckle 944 to connect the leg wrap 920 to the foot wrap 930 via the foot drop strap assembly 940. For example, the foot drop strap 942 may include a self-coupling hook-and-loop fastener. The foot drop strap 942 may be fed through the foot drop buckle 944 and then releasably coupled to itself to releasably couple the foot drop strap 942 to the foot drop buckle 944. In other embodiments, the foot drop strap 942 is coupled to the foot wrap 930 and the foot drop buckle 944 is coupled to the leg wrap 920. In still other embodiments, the foot drop strap assembly 940 does not include the foot drop buckle 944 and the foot drop strap 942 is releasably coupled to the foot wrap 930 with other means (e.g., a hook-and-loop fastener, a button fastener, an adhesive fastener, a clip fastener).

When the foot portion 56 of the lower limb 50 of the patient starts to drop during ambulation, the foot drop strap assembly 940 may prevent the foot portion 56 from dropping since the foot drop strap assembly 940 is coupled to the leg portion 52 of the lower limb 50 via the leg wrap 920. For example, when the foot portion 56 of the lower limb 50 of the patient starts to drop during ambulation, the foot wrap 930 may pull downwards on the foot drop strap assembly 940. Since the foot drop strap assembly 940 is coupled to the leg wrap 920 which is coupled to the leg portion 52, the foot drop strap assembly 940 prevents the foot portion 56 from angling forward relative to the leg portion 52. The foot drop strap assembly 940 may be configured to prevent the foot portion 56 from reaching a position where an angle between the foot portion 56 and the leg portion 52 exceeds an angle threshold. For example, if the angle threshold is ninety degrees, the foot drop strap assembly 940 may prevent the angle between the foot portion 56 and the leg portion 52 from exceeding ninety degrees to prevent the foot portion 56 from dropping forward.

In some embodiments, the foot drop strap assembly 940 is configured to be adjustable to allow for the angle threshold of the foot drop strap assembly 940 to be changed. To change the angle threshold of the foot drop strap assembly 940, a length of the foot drop strap assembly 940 between the leg wrap 920 and the foot wrap 930 may be changed. For example, when the foot drop strap 942 includes the self-coupling hook-and-loop fastener, the foot drop strap 942 may be fed through the foot drop buckle 944 and coupled to itself at a first position that results in a first length of the foot drop strap assembly 940 between the leg wrap 920 and the foot wrap 930 or at a second position that results in a second length of the foot drop strap assembly 940 between the leg wrap 920 and the foot wrap 930 that is greater than the first length. Since the second length is greater than the first length, a first angle threshold of the foot drop strap assembly 940 with the first length will be smaller than a second angle threshold of the foot drop strap assembly 940 with the second length since the foot portion 56 will be able to rotate further when the foot drop strap assembly 940 has the second length. When the foot drop strap assembly 940 is configured to be adjustable, the configuration of the foot drop assembly 900 may be changed based on the abilities of the patient. For example, the foot drop assembly 900 may be configured to have a lower angle threshold for a first patient with greater concerns of foot drop issues during ambulation than a second patient with less concerns of foot drop issues during ambulation.

As shown in FIGS. 34, and 36-42, the foot drop assembly 900 includes a pad assembly 950 releasably coupled to the heel support portion 914 and/or the leg wrap 920 and configured to cushion and/or support the lower limb 50, according to some embodiments. The pad assembly 950 may be positioned between the surface where the patient is positioned and the lower limb 50 of the patient to support the lower limb 50. In some embodiments, a position of the pad assembly 950 relative to the heel support portion 914 and/or the leg wrap 920 is adjustable. For example, the pad assembly 950 may be positioned lower or higher along the leg portion 52 of the lower limb 50 based on a portion of the lower limb 50 that needs to be cushioned and/or supported. In some embodiments, the pad assembly 950 has a patient facing surface (e.g., a top surface) of approximately 10 square inches. The pad assembly 950 may be formed out of a memory foam (e.g., a viscoelastic foam). The memory foam may be a highly supportive foam that is configured to conform to a profile (e.g., a shape) of the lower limb 50 when supporting the lower limb 50 to evenly distribute weight, alleviate pressure points, and improve blood circulation of the lower limb 50. The memory foam may also be a breathable foam configured dissipate heat received from the lower limb 50, which may prevent heat injuries to the lower limb 50. In various embodiments, the foot drop assembly 900 is used with other pads of the lower limb positioning system 10 (e.g., components of the wedge cushioning system 700, components of the universal fit positioning system 500, components of the modular positioning system 600).

### Overview of Temperature Regulation System

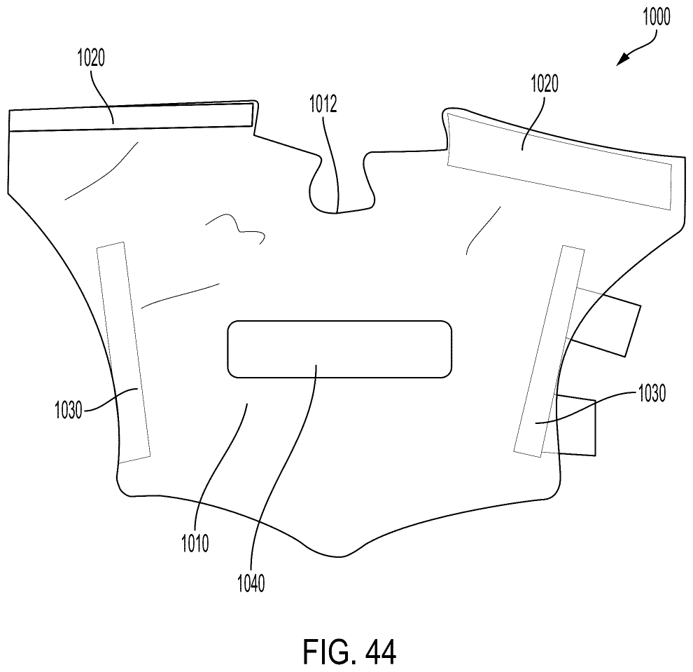

As shown in FIGS. 43A-44, the lower limb positioning system 10 includes a temperature regulation system 1000 (e.g., a cooling system, a heating system, a fluid temperature regulation system) configured to regulate a temperature of the lower limb 50 of the patient, according to some embodiments. By regulating the temperature of the lower limb 50 of the patient, the temperature regulation system 1000 may prevent overheating of the lower limb 50, which may lead to pressure injuries in the lower limb 50. Additionally, the regulation of the temperature of the lower limb 50 of the patient may promote blood flow through the lower limb 50.

As shown in FIGS. 43A-44, the temperature regulation system 1000 includes a fluid bladder 1010 defining an opening configured to receive a fluid from a temperature regulation pump (e.g., a temperature regulation device, a cooling pump, a temperature regulated pump, a fluid pumping system,). The fluid bladder 1010 may be formed from multiple layers of material configured to transfer heat between the fluid in the fluid bladder 1010 and the lower limb 50 of the patient. For example, the fluid bladder 1010 may be formed from a first layer of polyester fabric on an interior of the fluid bladder 1010 configured to retain the fluid in the opening of the fluid bladder, a second layer of foam insulation configured to retain heat, and a third layer of polyurethane on an exterior of the fluid bladder 1010 that may be easily cleaned. In some embodiments, the fluid bladder 1010 includes an interior side configured to be positioned proximate the lower limb 50 of the patient and an exterior side configured to be positioned away from the lower limb 50 of the patient. For example, the interior side of the fluid bladder 1010 may include less insulation (e.g., less of an insulating material) than the exterior side of the fluid bladder 1010 such that heat transfer between the interior side of the fluid bladder 1010 and the lower limb 50 of the patient is less inhibited than heat transfer between the exterior side of the fluid bladder 1010 and an environment surrounding the patient. In some embodiments, the fluid bladder 1010 may be formed out of Altrix wrap material. The fluid bladder 1010 may be formed out of a non-adhesive material.

The fluid pumping system may include a pump configured to provide the fluid to the fluid bladder 1010, a cooling system configured to decrease a temperature of the fluid provided to the fluid bladder 1010, and a heating system configured to increase a temperature of the fluid provided to the fluid bladder 1010. For example, the temperature regulation pump may be configured to provide the fluid to the opening of the fluid bladder 1010 at a temperature to cool or heat the lower limb 50 of the patient. In some embodiments, the temperature regulation pump may be a Stryker Altrix machine configured to supply a flow of water to the opening of the fluid bladder 1010 at a first temperature to cool the lower limb 50 of the patient and at a second temperature higher than the first temperature to heat the lower limb 50 of the patient.

As shown in FIGS. 43A-44, the fluid bladder 1010 is configured to wrap around the lower limb 50 of the patient, according to some embodiments. The fluid bladder 1010 may be configured to match a profile of the lower limb 50 in order to contact the lower limb 50 to transfer heat between the lower limb 50 and the fluid contained in the opening of the fluid bladder 1010. For example, the fluid bladder 1010 may be generally boot shaped to match the profile of the foot portion 56 of the lower limb 50. When generally boot shaped, the fluid bladder 1010 may be configured to be positioned on the lower limb 50 and/or positioned around other components of the lower limb positioning system 10 (e.g., the heel wrap assembly 708 of the wedge cushioning system 700, the dressing boot assembly 800, the foot drop assembly 900) to regulate the temperature of the lower limb 50 when the other components of the lower limb positioning system 10 are positioned on the lower limb 50. In other embodiments, the fluid bladder 1010 may be configured to be received by other components of the lower limb positioning system 10. For example, the fluid bladder 1010 may be configured to be received by at least one of the pockets 130 of the cushioning boot 100 in order to regulate the temperature of the lower limb 50 when the cushioning boot 100 is positioned on the lower limb 50.

As shown in FIGS. 43A and 44, the fluid bladder 1010 defines a heel opening 1012 configured to align with the heel portion 58 of the lower limb 50 of the patient when the fluid bladder 1010 is positioned on the lower limb 50 such that a caregiver can inspect the heel portion 58 without removing the fluid bladder 1010 from the lower limb 50. Additionally, the heel opening 1012 may allow for a caregiver to position the fluid bladder 1010 quickly and easily on the lower limb 50 of the patient by indicating the position and/or the orientation of the fluid bladder 1010 relative to the heel portion 58. The heel opening 1012 may be centered along a bottom of the fluid bladder 1010.

As shown in FIGS. 43A-44, the temperature regulation system 1000 includes a plurality of sole attachment portions 1020 configured to couple to each other to form a base portion of the fluid bladder 1010 configured to be positioned under the sole of the foot portion 56 of the lower limb 50 of the patient. A first of the sole attachment portions 1020 may be positioned on a left bottom side of the fluid bladder 1010 and a second of the sole attachment portions 1020 may be positioned on a right bottom side of the fluid bladder 1010.

When the fluid bladder 1010 is wrapped around the lower limb 50 of the patient, the first of the sole attachment portions 1020 may be releasably coupled with the second of the sole attachment portions 1020 under the sole of the foot portion 56 of the lower limb 50 of the patient to couple the fluid bladder 1010 to the lower limb 50 and/or form a base of the fluid bladder 1010 configured to be positioned under the sole of the foot portion 56 of the lower limb 50 of the patient. For example, a first of the sole attachment portions 1020 on the left bottom side of the fluid bladder 1010 may be configured as a first portion of a hook-and-loop fastener (e.g., a hook portion, a loop portion) configured to engage a second of the sole attachment portions 1020 positioned on the right bottom side of the fluid bladder 1010 that is configured as a second portion of the hook-and-loop fastener (e.g., the loop portion when the first of the sole attachment portions 1020 is configured as the hook portion, the hook portion when the first of the sole attachment portions 1020 is configured as the loop portion). As other examples, the sole attachment portions 1020 may be portions of button fasteners, portions of adhesive fasteners, portions of clip fasteners, etc.

As shown in FIGS. 43A-44, the temperature regulation system 1000 includes a plurality of leg attachment portions 1030 configured to couple the fluid bladder 1010 to the leg portion 52 of the lower limb 50 of the patient. The leg attachment portions 1030 may be adjustable to allow for the fluid bladder 1010 to be coupled to different sizes of the leg portion 52 of the lower limb 50 of patients. A first of the leg attachment portions 1030 may be positioned on a left side of the fluid bladder 1010 and a second of the leg attachment portions 1030 may be positioned on a right side of the fluid bladder 1010. When the fluid bladder 1010 is wrapped around the lower limb 50 of the patient, the first of the leg attachment portions 1030 may be releasably coupled with the second of the leg attachment portions 1030 around the leg portion 52 of the lower limb 50 to couple the fluid bladder 1010 to the lower limb 50. For example, a first of the leg attachment portions 1030 on the left side of the fluid bladder 1010 may be configured as a first portion of a hook-and-loop fastener (e.g., a hook portion, a loop portion) configured to engage a second of the leg attachment portions 1030 positioned on the right side of the fluid bladder 1010 that is configured as a second portion of the hook-and-loop fastener (e.g., the loop portion when the first of the leg attachment portions 1030 is configured as the hook portion, the hook portion when the first of the leg attachment portions 1030 is configured as the loop portion). As other examples, the leg attachment portions 1030 may be portions of button fasteners, portions of adhesive fasteners, portions of clip fasteners, etc.

As shown in FIG. 44 the temperature regulation system 1000 includes a temperature sensor 1040 configured to generate sensor data associated with a temperature of the fluid contained in the opening of the fluid bladder 1010 and/or a temperature of the lower limb 50 of the patient when the fluid bladder 1010 is positioned on the lower limb 50. The temperature sensor 1040 may be flexible such that the temperature sensor 1040 can deform when the fluid bladder 1010 is wrapped around the lower limb 50 of the patient. In some embodiments, the temperature regulation pump that is fluidly coupled to the fluid bladder 1010 may be operated based on the sensor data received from the temperature sensor 1040. For example, the temperature regulation pump may automatically adjust operation to provide the fluid to the fluid bladder 1010 with a warmer or colder temperature based on the sensor data received from the temperature sensor 1040.

### Overview of Prone Cushioning Assembly

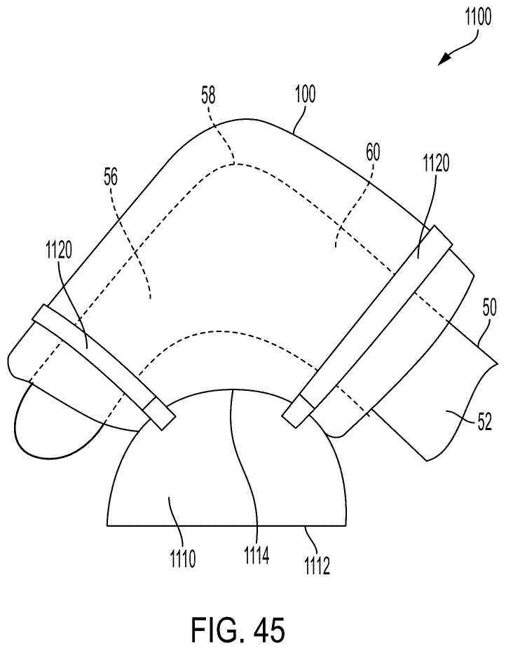

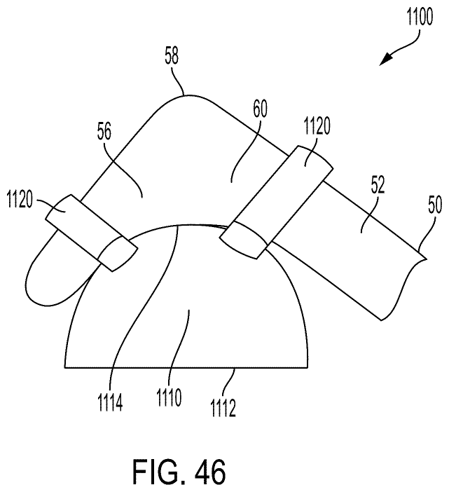

As shown in FIGS. 45 and 46, the lower limb positioning system 10 includes a prone cushioning assembly 1100 configured to cushion and/or support the lower limb 50 of the patient when the patient is in a prone position (e.g., in a position lying on their stomach), according to some embodiments. The prone cushioning assembly 1100 may be placed between a surface and the lower limb 50 when the patient is in the prone position to cushion and/or support the lower limb 50. For example, the prone cushioning assembly 1100 may be positioned between the lower limb 50 and the surface to prevent pressure injuries from forming on the lower limb 50 when the patient is in the prone position. In various embodiments, the prone cushioning assembly 1100 may cushion and/or support the lower limb 50 of the patient when the patient is in the prone position and other components of the lower limb positioning system 10 (e.g., cushioning boot 100, the heel wrap assembly 708 of the wedge cushioning system 700, the dressing boot assembly 800, the foot drop assembly 900) are positioned on the lower limb 50.

As shown in FIGS. 45 and 46, the prone cushioning assembly 1100 includes a prone pad 1110 (e.g., prone cushion) configured to be positioned between the surface and the lower limb 50 of the patient when the patient is in the prone position. The prone pad 1110 includes a bottom surface 1112 configured to contact the surface and a top surface 1114 configured to match a profile of a top of the foot portion 56 and a front of the leg portion 52 of the lower limb 50 that are oriented toward the surface when the patient is in the prone position so that the prone pad 1110 can support the foot portion 56 and the leg portion 52 when the patient is in the prone position. For example, the top surface 1114 may be rounded to match a rounded profile of the top of the foot portion 56 and the front of the leg portion 52 (e.g., proximate the ankle portion 60). The top surface 1114 may be configured to elevate the toes of the foot portion 56 when supporting the lower limb 50.

In some embodiments, the bottom surface 1112 is formed and/or coated with a material with a low coefficient of friction (e.g., lower than the surface under the patient). For example, the bottom surface 1112 may be formed and/or coated with nylon, silicone, nitrile, or other materials with coefficients of friction that are lower than standard bed sheets on a hospital bed. When the bottom surface 1112 is positioned on the surface and the lower limb 50 of the patient is supported by the prone pad 1110, the low coefficient of friction of the bottom surface 1112 may allow for adjustment of the position of the prone pad 1110 relative to the surface without having to lift the lower limb 50 off of the prone pad 1110. In some embodiments, the top surface 1114 is formed and/or coated with a material with a high coefficient of friction (e.g., higher than the surface under the patient, higher than the bottom surface 1112). For example, the top surface 1114 may be formed and/or coated with silicone rubber, neoprene, polyurethane foam, or other materials with coefficients of friction that are higher than standard bed sheets on a hospital bed. When the top surface 1114 supports the lower limb 50 of the patient, the high coefficient of friction of the top surface 1114 may prevent the lower limb 50 from sliding across the top surface 1114.

In some embodiments, the prone pad 1110 is formed and/or coated with materials that are easy to clean (e.g., materials that are compatible with cleaning products, materials that can be run through a laundry machine). For example, the prone pad 1110 may be formed and/or coated with silicone, nylon, polyester, or other materials that are easy to sterilize (e.g., chemically sterilize, sterilize using high temperatures), chemically resistant, and/or able to corrosion resistant. In some embodiments, the prone pad 1110 is formed and/or coated with materials that have antiseptic properties (e.g., materials that kill pathogens on contact, materials that are not conducing to hosting pathogens). For example, the prone pad 1110 may be formed and/or coated with silver nanoparticles, graphene oxide, or other materials that are detrimental to pathogens. In some embodiments, the prone pad 1110 is formed and/or coated with materials with a high durability (e.g., materials that can retain desired properties through multiple uses). For example, the prone pad 1110 may be formed and/or coated with polyester, Kevlar, nylon, or other materials that can withstand repeated uses. In some embodiments, the prone pad 1110 includes a cushioning material configured to distribute pressure around the lower limb 50 of the patient when the lower limb 50 are supported by the prone pad 1110 and the patient is in the prone position. For example, the prone pad 1110 may include polyurethane foam, polyethylene foam, silicone foam, memory foam, or other materials that provide cushioning and support. By cushioning the lower limb 50, the prone pad 1110 may prevent injuries to the lower limb 50 when the patient is in the prone position. In some embodiments, the prone pad 1110 is formed out of a memory foam (e.g., a viscoelastic foam).

As shown in FIGS. 45 and 46, the prone cushioning assembly 1100 includes a plurality of prone attachment straps 1120 coupled to the prone pad 1110 and configured to couple the prone pad 1110 to the lower limb 50, according to some embodiments. The prone attachment straps 1120 may be wrapped around the lower limb 50 to couple the lower limb 50 to the prone pad 1110. For example, a first of the prone attachment straps 1120 may be wrapped around the foot portion 56 of the lower limb 50 and a second of the prone attachment straps 1120 may be wrapped around the leg portion 52 of the lower limb 50. In other embodiments, the prone cushioning assembly 1100 includes one of the prone attachment straps 1120 configured to couple the prone pad 1110 to the lower limb 50. For example, the one of the prone attachment straps 1120 may be configured to wrap around the foot portion 56 or the leg portion 52 of the lower limb 50 to couple the prone pad 1110 to the lower limb 50.

In some embodiments, the prone attachment straps 1120 are configured as adjustable straps configured to be wrapped around the other components of the lower limb positioning system 10 (e.g., cushioning boot 100, the heel wrap assembly 708 of the wedge cushioning system 700, the dressing boot assembly 800, the foot drop assembly 900) when the other components of the of the lower limb positioning system 10 are positioned on the lower limb 50. For example, as shown in FIG. 45, the prone attachment straps 1120 may be configured to wrap around the cushioning boot 100 when the cushioning boot 100 is positioned on the lower limb 50 to couple the prone pad 1110 to the lower limb 50. In other embodiments, different configurations of the prone cushioning assembly 1100 may be configured to be used with different components of the lower limb positioning system 10. For example, a first of the prone cushioning assemblies 1100 may be configured to be used when no other components of the lower limb positioning system 10 are positioned on the lower limb 50, a second of the prone cushioning assemblies 1100 may be configured to be used when the cushioning boot is positioned on the lower limb 50, and a third of the prone cushioning assemblies 1100 may be configured to be used when the foot drop assembly 900 is positioned on the lower limb 50.

### Overview of Control System

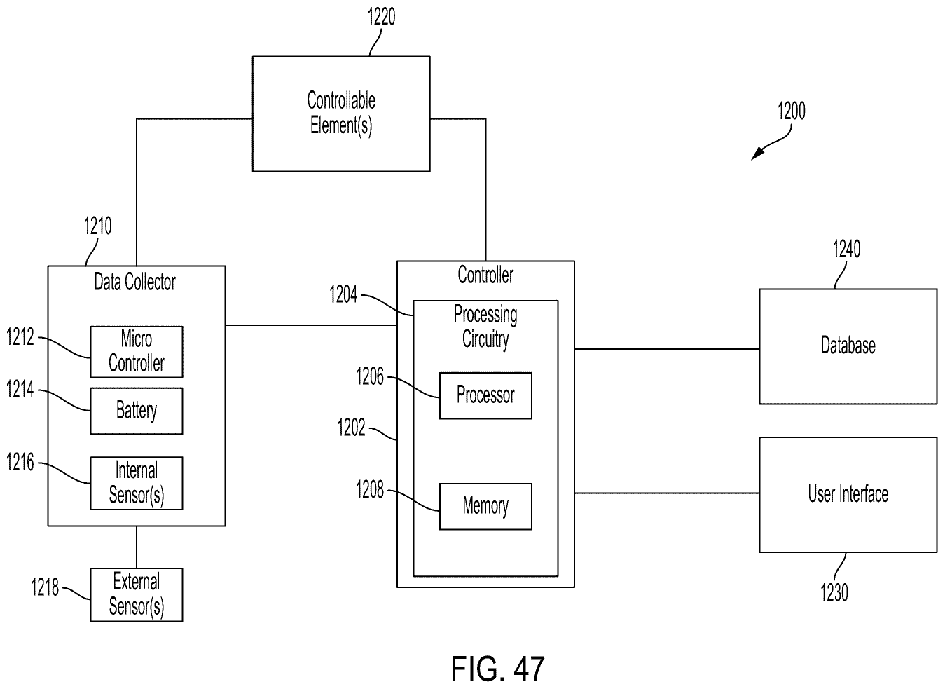

As shown in FIG. 47, the lower limb positioning system 10 includes a control system 1200 including a controller 1202, according to some embodiments. The controller 1202 may be configured to receive data associated with the components of the lower limb positioning system 10. In some embodiments, the controller 1202 is configured to operate the components of the lower limb positioning system 10. For example, the controller 1202 may be configured as the controller 312 configured to operate the compressor system 310. In some embodiments, the controller 1202 may be integrated into at least one of the components of the lower limb positioning system 10. In other embodiments, the controller 1202 is positioned on a bed associated with the patient, a mobile device associated with a caregiver of the patient, or another specialized device associated with the patient (e.g., a hospital system, a clinic system). In some embodiments, the controller 1202 may be the controller 312 of the compressor system 310.

As shown in FIG. 47, the controller 1202 includes processing circuitry 1204 including a processor 1206 and memory 1208. The processing circuitry 1204 can be communicably connected with a communications interface of controller 1202 such that the processing circuitry 1204 and the various components thereof can send and receive data via the communications interface. The processor 1206 can be implemented as a general purpose processor, an application specific integrated circuit (ASIC), one or more field programmable gate arrays (FPGAs), a group of processing components, or other suitable electronic processing components.

The memory 1208 (e.g., memory, memory unit, storage device) can include one or more devices (e.g., RAM, ROM, Flash memory, hard disk storage) for storing data and/or computer code for completing or facilitating the various processes, layers and modules described in the present application. The memory 1208 can be or include volatile memory or non-volatile memory. The memory 1208 can include database components, object code components, script components, or any other type of information structure for supporting the various activities and information structures described in the present application. According to some embodiments, the memory 1208 is communicably connected to the processor 1206 via the processing circuitry 1204 and includes computer code for executing (e.g., by at least one of the processing circuitry 1204 or the processor 1206) one or more processes described herein.

As shown in FIG. 47, the control system 1200 includes at least one data collector 1210 configured to collect data associated with the lower limb positioning system 10 and provide the data the controller 1202. In some embodiments, the data collector 1210 may be integrated into at least one of the components of the lower limb positioning system 10. For example, the data collector 1210 may be positioned inside of one of the pockets 130 of the cushioning boot 100. As other examples, the controller 1202 may positioned on or coupled to the boot cover 400, the universal fit positioning system 500, the modular positioning system 600, the wedge cushioning system 700, the dressing boot assembly 800, the foot drop assembly 900, and/or the temperature regulation system 1000.

In some embodiments, the data collector 1210 is electrically coupled to the controller 1202. For example, when the data collector 1210 is electrically coupled to the controller 1202, the data collector 1210 may provide the data to the controller 1202 electrically. In various embodiments, the data collector 1210 may be selectively electrically coupled to the controller 1202. For example, the data collector 1210 may not be electrically coupled to the controller 1202 while the data collector 1210 collects the data associated with the lower limb positioning system 10 and when provide the data to the controller 1202 electrically when the data collector 1210 is electrically coupled to the controller 1202. In other embodiments, the data collector 1210 can communicate with the controller 1202 through wireless communication (e.g., a network, Wi-Fi, Bluetooth, a local area network (LAN), a controller area network (CAN)) For example, the data collector 1210 may be communicatively coupled to the controller 1202 via a LAN and may provide the data to the controller 1202 through the LAN.

As shown in FIG. 47, the data collector 1210 includes a microcontroller 1212, a battery 1214 (e.g., a disposable battery, a rechargeable battery) configured to provide electrical power to the components of the data collector 1210, and at least one internal sensor 1216 configured to generate sensor data associated with the lower limb positioning system 10 and provide the sensor data to the microcontroller 1212, according to some embodiments. The microcontroller 1212 includes processing circuitry including a processor and memory. The internal sensors 1216 may be electrically and/or wirelessly connected to the microcontroller 1212 to provide the sensor data generate by the internal sensors 1216 to the microcontroller 1212. In other embodiments, the data collector 1210 does not include the internal sensors 1216 (e.g., when all of the sensors of the lower limb positioning system 10 are external to the data collector 1210).

In some embodiments, the data collector 1210 may include a case that defines an opening configured to receive the microcontroller 1212, the battery 1214, and/or the internal sensors 1216. The case may include an access door configured to provide access to the opening of the case in order to access the microcontroller 1212, the battery 1214, and/or the internal sensors 1216. For example, an operator of the data collector 1210 may open the access door to change the battery 1214. The case may be a reusable case. For example, the case may be configured to withstand cleaning chemicals typically used in a hospital environment. In some embodiments, the case may have dimensions approximately equal to 3.5″×2″×1″.

As shown in FIG. 47, the control system 1200 includes at least one external sensor 1218 configured to generate sensor data associated with the lower limb positioning system 10 and provide the sensor data to the data collector 1210, according to some embodiments. The external sensors 1218 may be electrically and/or wirelessly connected to the data collector 1210 to provide the sensor data generated by the external sensors 1218 to the data collector 1210. In some embodiments, the external sensors 1218 may be integrated into at least one of the components of the lower limb positioning system 10. For example, when the data collector 1210 is received by a first of the pockets 130 of the cushioning boot 100, the external sensor 1218 may be received by a second of the pockets 130 of the cushioning boot 100. The first of the pockets 130 may be positioned proximate the toes of the foot portion 56 of the lower limb 50 when the cushioning boot 100 is positioned on the lower limb 50 and the second of the pockets 130 may be positioned proximate the heel portion 58 of the lower limb 50 such that the external sensor 1218 is positioned proximate the heel portion 58 and can generate sensor data associated with the heel portion 58 (e.g., pressure data).

In some embodiments, the external sensors 1218 and the data collector 1210 may be positioned and/or coupled to different components of the lower limb positioning system 10 and/or different sub-components of the lower limb positioning system 10. For example, the data collector 1210 may be positioned on the cushioning boot 100 and the external sensor 1218 may be positioned on the temperature regulation system 1000. As another example, the data collector 1210 may be coupled to the wedge assembly 702 of the wedge cushioning system 700 and the external sensor 1218 may be coupled to the heel wrap assembly 708 of the wedge cushioning system 700. In other embodiments, the lower limb positioning system 10 does not include the external sensor 1218 (e.g., when all of the sensors of the lower limb positioning system are internal to the data collector 1210).

The sensor data generated by the internal sensors 1216 and/or the external sensors 1218 correspond to various states and function of the lower limb positioning system 10 and/or the lower limb 50 of the patient. For example, the internal sensors 1216 and/or the external sensors may include accelerometers, tachometers, gyroscopes, speedometers, GPS devices/sensors, temperature sensors, moisture sensors, voltmeters, ammeters, radar sensors, pressure sensors, tactile sensors, strain gauges photodetectors, motion sensors, piezoelectric sensors, myoelectric sensors, and/or proximity sensors amount other possible sensors and/or devices. The internal sensors 1216 and/or the external sensors 1218 may provide, to the data collector 1210, condition data, status information, and/or telemetry data associated with the lower limb positioning system 10 and/or the lower limb 50 of the patient. For example, the internal sensors 1216 and/or the external sensors 1218 may include a gyroscope configured to generate orientation data associated with the lower limb positioning system 10 and/or the lower limb 50 of the patient, a pressure sensor configured to generate pressure data associated with a pressure on the heel portion 58 of the lower limb 50 of the patient, a temperature sensor configured to generate sensor data associated with a temperature of the foot portion 56 of the lower limb 50 of the patient, etc. The data collector 1210 and/or the controller 1202 may utilize the sensor data provided by the internal sensors 1216 and/or the external sensors 1218 to determine conditions of the lower limb positioning system 10 and/or the lower limb 50 of the patient. For example, if the internal sensors 1216 and/or the external sensors 1218 provide the pressure data associated with the pressure on the heel portion 58 of the lower limb 50 of the patient and the temperature data associated with the temperature of the foot portion 56 of the lower limb 50 of the patient, the data collector 1210 and/or the controller 1202 may determine the pressure on the heel portion 58 of the lower limb 50 of the patient and the temperature of the foot portion 56 of the lower limb 50 of the patient.

The data collector 1210 and/or the controller 1202 are configured to receive (e.g., acquire) the sensor data (e.g., speed data, orientation data, sensor data, operating characteristics) from the internal sensors 1216 and/or the external sensors 1218, according to some embodiments. When the data collector 1210 receives the sensor data from the internal sensors 1216 and/or the external sensors 1218, the data collector 1210 may provide the sensor data to the controller 1202. In some embodiments, the data collector 1210 and/or the controller 1202 are configured to receive the sensor data from the sensor 316 of the compressor system 310. For example, the data collector 1210 and/or the controller 1202 may receive pressure data from the sensor 316 indicating the pressure applied by the compression cell 340 on the lower limb 50 of the patient. In some embodiments, the data collector 1210 and/or the controller 1202 are configured to receive the sensor data from the temperature sensor 1040 of the temperature regulation system 1000. For example, the data collector 1210 and/or the controller may receive temperature data from the temperature sensor 1040 indicating the temperature of the fluid within the fluid bladder 1010 and/or the temperature of the lower limb 50 of the patient.

The data collector 1210 and/or the controller 1202 may be configured to provide control outputs (e.g., control decisions, control signals) to at least one controllable component 1220 based on the sensor data received from the internal sensors 1216 and/or the external sensors 1218. The controllable components 1220 may include controllable components of the lower limb positioning system 10 (e.g., the mechanical stimulators 210, the compressor system 310) or other controllable components associated with the patient. For example, the controllable components may include an audio device configured to emit an audio alarm. The data collector 1210 and/or the controller 1202 may operate automatically based on programing or may use some combination of automatic and manual operation.

In some embodiments, the data collector 1210 and/or the controller 1202 may provide control outputs to the compressor system 310 based on the sensor data received from the internal sensors 1216, the external sensors 1218, and/or the sensor 316. For example, in response to receiving pressure data indicating that a pressure applied by the compression cell 340 on the lower limb 50 of the patient is below a pressure threshold, the data collector 1210 and/or the controller 1202 may provide a control output to the compressor 314 of the compressor system 310 to provide additional pressurized fluid to the compression cell 340 to increase the pressure applied on the lower limb 50 of the patient. In some embodiments, the data collector 1210 and/or the controller 1202 may provide control outputs to the temperature regulation pump of the temperature regulation system 1000 based on the sensor data received from the internal sensors 1216, the external sensors 1218, and/or the temperature sensor 1040. For example, in response to receiving temperature data indicating that a temperature of the lower limb 50 of the patient is above a temperature threshold, the data collector 1210 and/or the controller 1202 may provide a control output to the temperature regulation pump to decrease a temperature of the fluid provided to the fluid bladder 1010 to decrease the temperature of the lower limb 50 of the patient. In some embodiments, the data collector 1210 and/or the controller 1202 provide control outputs to the mechanical stimulators 210 of the mechanical stimulation system 200. The data collector 1210 and/or the controller 1202 may provide the control outputs to the mechanical stimulators 210 based on the sensor data received from the internal sensors 1216 and/or the external sensors 1218. For example, the data collector 1210 and/or the controller 1202 may provide a control output to the mechanical stimulators 210 operate the mechanical stimulators 210 to stimulate the lower limb 50 of the patient in response to receiving sensor data indicating that the lower limb 50 has moved less than a movement threshold.

In response to the sensor data, the data collector 1210 and/or the controller 1202 may determine that the patient is attempting to exit a bed and provide control outputs to an alarm system in order generate an alarm indicating that the patient is attempting to exit the bed. The data collector 1210 and/or controller 1202 may store exit thresholds that correspond with a patient attempting to exit a bed. For example, the exit thresholds may include an orientation threshold of the lower limb 50, a movement threshold of the lower limb 50, a pressure threshold of a pressure on the lower limb 50, or thresholds related to the sensor data. The data collector 1210 and/or the controller 1202 may determine that the patient is attempting to exit the bed when the sensor data exceeds at least one of the exit thresholds and provide the control output to the alarm system to generate the alarm. The alarm generated by the alarm system may include an alarm notification provided to a user device (e.g., a cell phone, a computer) of a caregiver and/or an audio alarm provided to the patient in order to communicate the unsafe conditions to the patient.

In response to the sensor data, the data collector 1210 and/or the controller 1202 may generate data and/or control outputs associated with ambulation of the patient. For example, the data collector 1210 and/or the controller 1202 may determine a number of steps taken by the patient during ambulation based on acceleration data included in the sensor data. As another example, the data collector 1210 and/or the controller 1202 may store fall thresholds that corresponds with a patient falling and the data collector 1210 and/or the controller 1202 may generate a control output associated with a fall in response to the sensor data exceeding the fall thresholds. The control output associated with the fall may be provided to an alarm system to generate an alarm associated with the fall.

In response to the sensor data, the data collector 1210 and/or the controller 1202 may monitor temperature conditions and/or moisture conditions of the lower limb 50 of the patient indicated by the sensor data and provide an evaluation associated with the lower limb 50 of the patient. The evaluation may include recommendations associated with the care of the patient. For example, if a temperature condition associated with the lower limb 50 of the patient exceeds a temperature threshold, the evaluation may include a recommendation to cool down the lower limb 50 of the patient. In some embodiments, the evaluation may include a prediction of a pressure injury forming on the lower limb 50 of the patient. For example, the data collector 1210 and/or the controller 1202 may utilize historical temperature conditions and/or moisture conditions to predict that a pressure injury will occur to the lower limb 50 of the patient. The evaluation may also include a treatment plan for preventing the pressure injury from forming on the lower limb 50 of the patient. For example, the treatment plan may include positioning the cushioning boot 100 on the lower limb 50 of the patient to prevent the pressure injury from forming on the lower limb 50 of the patient. In some embodiments, the data collector 1210 and/or the controller 1202 may feed the sensor data into pressure mapping algorithms (e.g., to generate a pressure map) or other pressure associated algorithms in order to predict the pressure injury forming on the lower limb 50 of the patient.

In response to the sensor data, the data collector 1210 and/or the controller 1202 may provide feedback in relation to exercise and/or movement of the patient. The data collector 1210 and/or the controller 1202 may determine an actual exercise or movement of the patient based on the sensor data and compare the actual exercise or movement of the patient with an ideal exercise or movement. The comparison may be provided to the patient and/or the caregiver of the patient so that the patient and/or the caregiver can improve future exercises and/or movements of the patients. In some embodiments, the data collector 1210 and/or the controller 1202 may compare the exercise and/or movement of the patient with goals set by the patient and/or the caregiver and provide the comparison with the goals to the patient and/or the caregiver.

In response to the sensor data, the data collector 1210 and/or the controller 1202 may provide the sensor data as game results (e.g., in a gamified manner) to the patient. For example, the data collector 1210 and/or the controller 1202 may associate movement of the lower limb 50 of the patient with a game associated with movements of lower limbs. In response to the sensor data indicating that the movement of the lower limb 50 exceeds a game threshold, the data collector 1210 and/or the controller 1202 may provide a positive game result to the patient (e.g., notifying the patient that they won the game). In response to the sensor data indicating that the movement of the lower limb 50 does not exceed the game threshold, the data collector 1210 and/or the controller 1202 may provide a negative game result to the patient (e.g., notifying the patient that they lost the game). By providing the sensor data as game results, the patient may be motivated to achieve positive game results, which may positively affect the health of the patient.

In some embodiments, the data collector 1210 is configured to generate low level control outputs associated with low level data analysis of the sensor data and the controller 1202 is configured to generate high level control outputs associated with high level data analysis of the sensor data. For example, the data collector 1210 may be configured to generate a low level control output associated with the audio device to emit the audio alarm in response to the temperature data indicating that a temperature of the lower limb 50 of the patient is above a temperature threshold. As another example, the controller 1202 may be configured to generate a high level control output associated with a care treatment plant of the patient in response to the pressure data indicating that a pressure applied on the heel portion 58 of the lower limb 50 of the patient is following a trend that indicates a pressure injury may be forming on the heel portion 58. By generating the low level control outputs with the data collector 1210 and the high level control outputs with the controller 1202, the processing power of the data collector 1210 can be reduced while still providing time critical control outputs that may not be able to wait until the sensor data is provided to the controller 1202.

As shown in FIG. 44, the controller 1202 may operate a user interface 1230 to provide interfaces associated with the sensor data to a user of the user interface 1230. In some embodiments, the user interface 1230 may be associated with a user device (e.g., a computer, a cell phone, a tablet). For example, the controller 1202 may operate the user interface 1230 to provide a trend interface to the user associated with trends in the sensor data over time. As another example, the controller 1202 may operate the user interface 1230 to provide a feedback interface associated with comparisons of movements and/or exercises of the patient based on the sensor data. As another example, the controller 1202 may operate the user interface 1230 to provide a treatment plan determined based on the sensor data. As another example, the controller 1202 may operate the user interface 1230 to provide a gamification interface associated with the gamification of the sensor data.

As shown in FIG. 44, the controller 1202 may provide the sensor data to a database 1240. The database 1240 may be configured to store the sensor data under an index associated with an identification of the patient associated with the sensor data. The index may also include other data associated with the patient such as demographics of the patient. The database 1240 may store historical data associated with the patient such as sensor data associated with previous medical events, medical data, etc. In some embodiments, the controller 1202 may utilize the data stored in the database 1240 to generate the control outputs associated with the sensor data. For example, the controller 1202 may utilize the sensor data received from the internal sensor 1216 and/or the external sensor 1218 and medical history data received from the database 1240 when generating a treatment plan for a patient.

### Configuration of Example Embodiments

As utilized herein, the terms “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “example” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the FIGURES. It should be noted that the orientation of various elements may differ according to other example embodiments, and that such variations are intended to be encompassed by the present disclosure.

It is important to note that the construction and arrangement of the systems, apparatuses, and methods shown in the various example embodiments is illustrative only. Additionally, any element disclosed in one embodiment may be incorporated or utilized with any other embodiment disclosed herein. For example, any of the example embodiments described in FIGS. 1-47 of this application can be incorporated with any of the other example embodiment described. Although only one example of an element from one embodiment that can be incorporated or utilized in another embodiment has been described above, it should be appreciated that other elements of the various embodiments may be incorporated or utilized with any of the other embodiments disclosed herein.

What is claimed is:

1. A wedge cushioning system for supporting a lower limb of a patient, the wedge cushioning system comprising:
a wedge assembly comprising:
a wedge pad configured to cushion the lower limb of the patient,
a first wedge wing pivotably coupled to a first side of the wedge pad and selectively pivotable between a first lowered position and a first raised position,
a second wedge wing pivotably coupled to a second side of the wedge pad and selectively pivotable between a second lowered position and a second raised position independent of the first wedge wing, the second side opposite the first side, and
a strap configured to be releasably coupled between the first wedge wing and the second wedge wing.

2. The wedge cushioning system of claim 1, wherein the first wedge wing, the second wedge wing, and the strap are configured such that when the strap is coupled between the first wedge wing and the second wedge wing, the first wedge wing is in the first raised position and the second wedge wing is in the second raised position.

3. The wedge cushioning system of claim 1, wherein:
the wedge pad comprises a wedge top surface;
the first wedge wing comprises a first wing inside surface;
the second wedge wing comprises a second wing inside surface; and
the wedge pad, the first wedge wing, and the second wedge wing are configured such that the wedge top surface is co-planar with the first wing inside surface and the second wing inside surface when the first wedge wing is in the first lowered position and the second wedge wing is in the second lowered position.

4. The wedge cushioning system of claim 3, wherein the first side of the wedge pad and the second side of the wedge pad are oriented perpendicular to the wedge top surface.

5. The wedge cushioning system of claim 3, wherein:
the first wedge wing comprises a first wing outside surface opposing the first wing inside surface;
the second wedge wing comprises a second wing outside surface opposing the second wing inside surface; and
the strap is configured to be releasably coupled between the first wing outside surface and the second wing outside surface.

6. The wedge cushioning system of claim 3, wherein the wedge pad, the first wedge wing, and the second wedge wing are configured such that the first wing inside surface and the second wing inside surface face each other when the first wedge wing is in the first raised position and the second wedge wing is in the second raised position.

7. The wedge cushioning system of claim 1, wherein the wedge pad comprises:
a wedge top surface having a first coefficient of friction; and
a wedge bottom surface opposing the wedge top surface, the wedge bottom surface having a second coefficient of friction that is less than the first coefficient of friction.

8. The wedge cushioning system of claim 7, wherein:
the first wedge wing comprises a first wing inside surface having the first coefficient of friction; and
the second wedge wing comprises a second wing inside surface having the first coefficient of friction.

9. The wedge cushioning system of claim 1, wherein:
the wedge assembly comprises a wedge tip end and a wedge base end;
a height of the wedge pad increases in a direction from the wedge tip end towards the wedge base end; and
a width of the strap in the direction from the wedge tip end towards the wedge base end is less than a wing length of the first wedge wing in the direction from the wedge tip end towards the wedge base end.

10. The wedge cushioning system of claim 1, further comprising:
a heel wrap assembly configured to wrap around the lower limb of the patient, the heel wrap assembly comprising:
a wrap base portion,
a wrap heel portion extending from the wrap base portion, and
a plurality of wrap arms extending from the wrap base portion, the wrap arms configured to be wrapped around the lower limb to couple the heel wrap assembly to the lower limb;
wherein the heel wrap assembly is configured to be positioned between the wedge pad and the lower limb of the patient.

11. The wedge cushioning system of claim 10, wherein the wedge pad, the first wedge wing, and the second wedge wing are configured as reusable elements; and
wherein the heel wrap assembly and the strap are configured as disposable elements.

12. The wedge cushioning system of claim 10, wherein the heel wrap assembly further comprises:
a grip element configured to be positioned between the wrap heel portion and the wedge pad, the grip element configured restrict movement between the wedge pad and the heel wrap assembly.

13. The wedge cushioning system of claim 1, wherein:
the wedge assembly comprises a wedge tip end and a wedge base end;
a height of the wedge pad increases in a direction from the wedge tip end towards the wedge base end; and
the first wedge wing comprises a first wing tip edge on an end of the first wedge wing positioned closest to the wedge tip end, the first wing tip edge extending away from the wedge tip end as the first wing tip edge extends away from the wedge pad.

14. The wedge cushioning system of claim 1, wherein:
the wedge assembly comprises a wedge tip end and a wedge base end;
a height of the wedge pad increases in a direction from the wedge tip end towards the wedge base end; and
the first wedge wing comprises a first wing tip edge on an end of the first wedge wing positioned closest to the wedge base end, the first wing tip edge extending towards from the wedge base end as the first wing tip edge extends away from the wedge pad.

15. A wedge cushioning system for supporting a lower limb of a patient, the wedge cushioning system comprising:
a wedge assembly configured to cushion the lower limb of the patient, the wedge assembly, the wedge assembly comprising a wedge top surface;
a sensor positioned on the wedge assembly configured to generate sensor data associated with the wedge assembly; and
a processing circuit comprising memory and one or more processors, the processing circuit configured to:
receive, from the sensor, the sensor data associated with the wedge assembly,
determine, based on the sensor data, a pressure applied on the wedge top surface of the wedge assembly, and
provide the pressure applied on the wedge top surface to a user interface associated with a caregiver.

16. The wedge cushioning system of claim 15, wherein the processing circuit is further configured to:
generate a pressure map associated with the pressure applied on the wedge top surface of the wedge assembly; and
provide the pressure map to the user interface associated with the caregiver.

17. The wedge cushioning system of claim 15, wherein the processing circuit is further configured to, responsive to the pressure applied on the wedge top surface exceeding a pressure threshold, operate an alarm system to generate an alarm associated with the pressure exceeding the pressure threshold.

18. The wedge cushioning system of claim 15, wherein the processing circuit is further configured to:
determine, based on the sensor data, that a pressure injury will form of the lower limb;
generate a notification associated with the pressure injury; and
provide the notification to the user interface associated with the caregiver.

19. The wedge cushioning system of claim 15, wherein the processing circuit is further configured to:
determine, based on the sensor data, that the lower limb is not supported by the wedge assembly; and
operate an alarm system to generate an alarm associated with the lower limb not being supported by the wedge assembly.

20. The wedge cushioning system of claim 15, wherein the wedge assembly further comprises:
a wedge pad configured to cushion the lower limb of the patient, the wedge pad comprising the wedge top surface;
a first wedge wing pivotably coupled to a first side of the wedge pad and pivotably between a first lowered position and a first raised position; and
a second wedge wing pivotably coupled to a second side of the wedge pad and pivotably between a second lowered position and a second raised position independent of the first wedge wing, the second side opposite the first side.
